(19) United States
(12) Patent Application Publication
(10) Pub. No.: US 2023/0042929 A1
(43) Pub. Date: Feb. 9, 2023

(54) Methods of Screening to Determine Effective Dosing of Cancer Therapeutics

(71) Applicant: Kiyatec, Inc., Greenville, SC (US)

(72) Inventors: Kate Appleton, Greenville, SC (US); Tessa Desrochers, Greenville, SC (US)

(21) Appl. No.: 17/771,863

(22) PCT Filed: Nov. 9, 2020
(86) PCT No.: PCT/US2020/059667

§ 371 Date: Apr. 26, 2022

Related U.S. Application Data
(60) Provisional application No. 62/933,339, filed on Nov. 8, 2019.

(51) Int. Cl. C12M 1/42; C12N 5/0783; C12N 5/09; G01N 33/50; G01N 33/574

(52) U.S. Cl. CPC C12M 35/08; C12N 5/0636; C12N 5/0693; G01N 33/5011; G01N 33/502; G01N 33/5047; G01N 33/505; G01N 33/57484; C12N 2503/04; G01N 2800/52

(57) ABSTRACT

The present application contemplates methods of screening therapeutic agents for treating cancer comprising co-culturing immune cells and tumor cells isolated from a subject under conditions that allow the immune cells and the tumor cells to form a cancer spheroid. The cancer spheroid may then be exposed to at least one therapeutic agent, and the responsiveness of the tumor cells the spheroid to the therapeutic agent may be measured.

### CROSS REFERENCE TO RELATED APPLICATIONS

The present application claims priority to the provisional application having U.S. Patent Application Ser. No. 62/933,339, filed on Nov. 8, 2019, the entire contents of which are incorporated herein by reference.

### TECHNICAL FIELD

The subject matter disclosed herein is generally directed to cell models for studying cancer.

### BACKGROUND

Knowledge of immune responses that correlate with clinical outcome is essential for the development of strategies to harness a patient's immune system to eradicate cancer. Pre-clinical platforms that recapitulate the immune response in the context of cancer are necessary for adequate understanding and detection of clinical efficacy, however, the technology to accurately test immuno-oncology (I/O) therapy response is lacking. Despite the value animal models provide in a pre-clinical setting, they lack human biology and matched patient tumor and immune cell interactions. To address this shortcoming, an in vitro 3D tissue model utilizing human tumor and matched immune cells was developed. This model maintains autologous patient tumor cells and immune cells for the testing and prediction of immune cell responses. These 3D tissue models recapitulate the patient tumor microenvironment and detect responses to I/O agents. Citation or identification of any document in this application is not an admission that such a document is available as prior art to the present invention.

### SUMMARY

In certain example embodiments, the invention comprises a method of screening therapeutic agents for treating cancer comprising co-culturing immune cells and tumor cells isolated from a subject under conditions that allow the immune cells and the tumor cells to form a cell mass, exposing the cell mass to at least one therapeutic agent, and measuring the responsiveness of the tumor cells in the cell mass to the at least one therapeutic agent.

In some embodiments, the method further comprises determining a ratio of immune cells to tumor cells prior to generating the 3D cancer cell model. In some embodiments, the ratio of tumor cells to immune cells is from 1:0.05 to 1:100, such as from 1:0.05 to 1:20. In specific embodiments, the ratio of tumor cells to immune cells is 1:10.

In some embodiments, the immune cells and the tumor cells are from the same subject.

In some embodiments, the immune cells comprise T cells, natural killer cells, dendritic cells, macrophages, or a combination thereof. In specific embodiments, the immune cells comprise T cells.

In some embodiments, the at least one therapeutic agent comprises at least one checkpoint inhibitor. In some embodiments, the checkpoint inhibitor targets PD-1.

In some embodiments, the at least one therapeutic agent further comprises at least one poly(ADP-ribose) polymerase inhibitor. In some embodiments, the at least one poly(ADP-ribose) polymerase inhibitor comprises olaparib, niraparib, rucaparib, talazoparib, or a combination thereof.

In some embodiments, the ratio of the checkpoint inhibitor to the PARP inhibitor can be from about 100:0 to about 0:100, such as from about 75:25 to about 25:75, such as about 50:50, or any ranges therebetween, depending on application and treatment.

In some embodiments, the cell mass is a tumor spheroid.

In some embodiments, the responsiveness of the tumor cells is a decrease in viability.

In some embodiments, the method further comprises identifying a patient-specific treatment based on the decrease in tumor cell viability.

In some embodiments, the at least one therapeutic agent induces secretion of TNF-α, MIP-1α, and IFNγ.

In some embodiments, the method further comprises using isolated immune cells for use in a cell therapy.

In another aspect, the invention comprises a 3D cancer cell model comprising immune cells and tumor cells isolated from a subject that are co-cultured under conditions that allow the immune cells and the tumor cells to form a cell mass.

In some embodiments, the ratio of tumor cells to immune cells is 1:0.05 to 1:100, such as from 1:0.05 to 1:20. In specific embodiments, the ratio of tumor cells to immune cells is 1:10.

In some embodiments, the immune cells and the tumor cells are from the same subject. In some embodiments, the immune cells and the tumor cells are from different subjects.

In some embodiments, the immune cells express an immune checkpoint protein selected from the group consisting of CTLA4, BTLA, LAG3, ICOS, PD-1, PDL1, KIR, CD40, OX40, CD137, GITR, CD27 and TIM-3. In specific embodiments, the immune checkpoint protein is PD-1.

In some embodiments, the cell mass is a tumor spheroid.

In yet another aspect, the invention also comprises a 3D system for co-culturing cells comprising a first compartment comprising immune cells isolated from a subject; a second compartment comprising tumor cells isolated from a subject; wherein the first and second compartments comprise a porosity that allows migration of immune cells between compartments; and wherein the first compartment and the second compartment are stacked relative to each other.

In some embodiments, the compartment comprising the immune cells is stacked on top of the compartment comprising the tumor cells. In some embodiments, the compartment comprising the tumor cells is stacked on top of the compartment comprising the immune cells.

In some embodiments, the immune cells are clonally expanded prior to being placed in the first compartment. In some embodiments, the tumor cells are clonally expanded prior to being placed in the second compartment. In some embodiments, both the immune cells and the tumor cells are clonally expanded prior to being placed in the respective compartments.

In some embodiments, the ratio of tumor cells to immune cells is 1:0.05 to 1:100, such as from 1:0.05 to 1:20. In specific embodiments, the ratio of tumor cells to immune cells is 1:10.

In some embodiments, the immune cells and the tumor cells are from the same subject. In some embodiments, the immune cells and tumor cells are from different subjects.

In some embodiments, the immune cells are from a healthy subject.

In some embodiments, the immune cells express an immune checkpoint protein selected from the group consisting of CTLA4, BTLA, LAG3, ICOS, PD-1, PDL1, KIR, CD40, OX40, CD137, GITR, CD27 and TIM-3.

In yet another aspect, the invention comprises a method of measuring the level of migration (e.g., the movement of cells from one compartment to another compartment) of immune cells in a 3D co-culture system comprising seeding immune cells isolated from a subject in a first compartment; seeding tumor cells isolated from a subject in a second compartment; culturing the immune cells and the tumor cells under conditions that allow migration of the immune cells from the first compartment to the second compartment; exposing at least the second compartment to at least one therapeutic agent; and measuring the responsiveness of the tumor cells to the at least one therapeutic agent.

In some embodiments, the compartment comprising the immune cells is stacked on top of the compartment comprising the tumor cells. In some embodiments, the compartment comprising the tumor cells is stacked on top of the compartment comprising the immune cells.

In some embodiments, migration of the immune cells from the first compartment to the second compartment allows the immune cells to form a cell mass with the tumor cells.

In some embodiments, the isolated immune cells are clonally expanded prior to seeding them in the first compartment. In some embodiments, the isolated tumor cells are clonally expanded prior to seeding them in the second compartment. In some embodiments, both the isolated immune cells and the isolated tumor cells are clonally expanded prior to seeding them in the first and second compartments.

In some embodiments, the ratio of tumor cells to immune cells is 1:0.05 to 1:100, such as 1:0.05 to 1:20. In specific embodiments, the ratio of tumor cells to immune cells is 1:10.

In some embodiments, the immune cells and the tumor cells are from the same subject. In some embodiments, the immune cells and the tumor cells are from different subjects.

In some embodiments, the immune cells express an immune checkpoint protein selected from the group consisting of CTLA4, BTLA, LAG3, ICOS, PD-1, PDL1, KIR, CD40, OX40, CD137, GITR, CD27 and TIM-3.

In some embodiments, the at least one therapeutic agent is an immune checkpoint inhibitor. In some embodiments, the immune checkpoint inhibitor targets PD-1.

In some embodiments, the at least one therapeutic agent further comprises at least one poly(ADP-ribose) polymerase inhibitor. In some embodiments, the at least one poly(ADP-ribose) polymerase inhibitor comprises olaparib, niraparib, rucaparib, talazoparib, or a combination thereof.

In some embodiments, the ratio of the checkpoint inhibitor to the PARP inhibitor can be from about 100:0 to about 0:100, such as from about 75:25 to about 25:75, such as about 50:50, or any ranges therebetween, depending on application and treatment.

In some embodiments, the immune checkpoint inhibitor is pembrolizumab.

In some embodiments, the cells in the second compartment are a cancer spheroid.

In some embodiments, the responsiveness comprises death of the tumor cells.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIGS. 1A-1F—Cytotoxic T-cell Mediated Tumor Spheroid Death. (1A) GEN24 tumor spheroid viability in the presence of increasing amounts of GEN24 T cells after 72 hours. A cell death control, 10% DMSO, was used for comparison. (1B) Viability of GEN24 T cells seeded alone at indicated cell numbers to reflect tested ratios. A cell death control, 10% DMSO, was used for each tested T cell density. (1C) GEN26 tumor spheroid viability in the presence of increasing amounts of GEN26 T cells after 24 hours. A cell death control, 10% DMSO, was used for comparison. (1D) Viability of GEN26 T cells seeded alone at indicated cell numbers to reflect tested ratios. A cell death control, 10% DMSO, was used for each tested T cell density. (1E) GEN22 tumor spheroid viability at a 1:10 tumor cell to T cell ratio after 72 hours. (1F) Viability of GEN22 T cells seeded alone to reflect tested 1:10 ratio. A cell death control, 10% DMSO, was used for comparison. Error bars represent standard deviation of seven technical replicates.

FIGS. 2A-2C—Detection of Tumor Spheroid-specific T cell Responses in a Personalized Manner. (2A) Basal levels of different T cell populations and after tumor spheroid stimulation for 24 hours was determined via flow cytometry. Error bars represent standard deviation of N=3 or 4 patients. (2B) Basal levels of activated cytotoxic T cells and after tumor spheroid stimulation for 24 hours was determined using flow cytometry and analyzed for GEN25 and GEN21. Error bars represent standard deviation of N=2 patients. Unpaired t test: not significant (n.s.) (2C) Basal levels of activated cytotoxic T cells and after tumor spheroid stimulation for 24 hours was determined using flow cytometry and analyzed for GEN19 and GEN26. Error bars represent standard deviation of N=2 patients. Unpaired t test: **p<0.01.

FIGS. 3A-3D—Detection of Pembrolizumab Occupancy and Pembrolizumab Induced Tumor Spheroid Death. (3A) Representative flow cytometry data of GEN25 T cells treated with increasing amounts of pembrolizumab for 72 hours. (3B) Pembrolizumab binding to T cells was determined by detecting percent PD-1+ T cells treated with increasing amounts of pembrolizumab for 72 hours. Error bars represent standard deviation of N=4 patients (GEN20, GEN21, GEN25, and GEN26). Unpaired One-way ANOVA ***p<0.005. (3C) Basal levels of PD-1+/CD69+ T cells compared to T cells stimulated with matched tumor spheroids with or without 100 μg/ml pembrolizumab. Error bars represent standard deviation of N=3 patients (GEN19, GEN21, and GEN26). Unpaired One-way ANOVA: **p<0.001. (3D) GEN26 tumor spheroids were seeded alone or in the presence of matched T cells and treated with or without 100 μg/ml pembrolizumab for 24 hours. Tumor spheroid viability was determined by measuring relative luminescence units (RLU) after 24 hours. Error bars represent standard deviation of six technical replicates.

FIGS. 4A-4C—Detection of T cell Infiltration in a Microtumor Model. (4A) Schematic of the microtumor model with the T cell compartment stacked above the microtumor. PKH+ gate and CD3+ gates were determined by analyzing T cell compartments following dissociation via flow cytometry. PKH+ gate was also determined by comparing un-labeled T cell to the PKH stained T cells (data not shown). (4B) Microtumors alone or (4C) cultured with above T cell compartments were analyzed for dual PKH+/CD3+ staining indicative of T cell infiltration into the microtumor. Two patients with patient-specific differences are depicted (GEN19 and GEN20).

FIGS. 5A-5C—Pembrolizumab Induces Analyte Secretion Which Correlates with Decreased Microtumor Growth Rate. (5A) Microtumor growth rate was determined via PrestoBlue Viability assays from day 1 to day 7 for 100 μg/ml pembrolizumab and no treatment. Fold change pembrolizumab treatment was compared to no treatment. The mean of two replicates for four patients is depicted. (5B) Fold change analyte secretion was determined at day 7 after 100 μg/ml pembrolizumab and compared to no treatment. The mean of two replicates for four patients is depicted. (5C) Pearson correlations were determined for pembrolizumab induced fold change in microtumor growth rate compared to pembrolizumab induced fold change in analyte secretion for TNF-α, IFNγ, MIP-1α,

FIG. 6—Secretion of analytes across four patients in the microtumor model. Depicted is the concentration of six tested analytes at day seven for microtumors cultured alone or co-cultured with matched T cells.

FIG. 7—Further data showing detection of T cell infiltration in a microtumor model.

FIG. 8—Percent infiltration of CD3+ T cells in tumor cells of various patients in the presence and absence of treatment with pembrolizumab.

FIG. 9—Schematic showing cytokine expression signatures in different patients in response to treatment with pembrolizumab.

FIG. 10—Model for possible adaptive immune resistance.

FIG. 11—Data showing detection of adaptive immune resistance response by tumor cells when they are treated with T cells. PD-L1 on the cells increases in the presence of T cells.

FIG. 12—Results of screening for most effective effector cell to tumor cell ratios.

FIG. 13—Results of therapy response using tumor infiltrating T lymphocytes. Data shown at 24 hours.

FIG. 14—A schematic of a 3D microtumor. Immune cells and tumor cells are placed in two separate compartments that are stacked relative to each other (left). Tumor cells recruit immune cells to form a cell mass (right).

FIG. 15—Schematic of a 3D microtumor similar to FIG. 14, but shown inside a perfusion bioreactor. In certain embodiments, the bioreactor could also be static. Co-culture of immune and tumor cells could be direct or segregated in various embodiments, and culture could progress over 10 to 64 days.

FIG. 16—Schematic outlining methodology for predicting patient response to checkpoint blockade therapy using in vitro 3D culture.

FIGS. 17A-17C—(17A) Effector cell (T-cell) to target cell (tumor cell) (E:T) ratio screens with T cells from healthy donors. (17B) T-cell mediated killing of tumor spheroids for two melanoma cell lines after 24 hour treatment with Pembrolizumab. (17C) Atezolizumab and Durvalumab tumor spheroid killing in the presence or absence of T-cells using a NSCLC cell line for 24 hours. Two-way ANOVA: *p<0.05, **p<0.001, n=7.

FIGS. 18A-18C—(18A) Pembrolizumab occupancy determined for T cells following 72 hrs of treatment. One-way ANOVA ***p<0.005, n=4. (18B) CD3+/PD-1−/CD69+ T cells compared to T cells stimulated with spheroids ±100 μg/mL pembrolizumab. One-way ANOVA: **p<0.001. Blue=Patient GEN26. (18C) GEN26 spheroids were seeded ± matched T cells and treated ±100 μg/mL pembrolizumab for 24 hrs. Error bars=standard deviation, n=6.

FIGS. 19A-19C—(19A) Tumor samples were dissociated into single cells and tested for PD-L1 expression using flow cytometry. (19B) T cell populations from within the dissociated tumor cells were analyzed for CD4+/CD25+ and for CD8+/CD69+. Unpaired t test from two independent experiments: **p<0.01, ***p<0.005 (19C) Spheroids were treated with or without 300 ug/mL of pembrolizumab and spheroid viability was determined after 24 hrs. One way ANOVA from two independent experiments: *p<0.05.

FIG. 20—Schematic outlining methodology generating an autologous 3D tumor spheroid model.

FIGS. 21A-21C—Cytotoxic T-cell Mediated Tumor Spheroid Death. (21A) GEN24 spheroid viability in the presence of increasing amounts of GEN24 T cells after 72 hrs. (21B) GEN26 spheroid viability in the presence of increasing amounts of GEN26 T cells after 24 hrs. (21C) GEN22 spheroid viability at a 1:10 tumor cell to T cell ratio after 72 hrs. T-cell only spheroids were assessed for all patient samples to verify viability. CellTiter Glo 3D® was used to determine viability for all experiments. Error bars=SD. Seven replicates per patient was tested.

FIGS. 22A-22G—Detection of T-cell population shifts. (22A) T cells ± spheroids for 24 hrs determined via flow. (22B and 22C) Activated cytotoxic Tcells ± spheroids for 24 hrs determined using flow. t test: not significant (n.s.); **p<0.01. (22D) Flow cytometry data of T cells treated with increasing amounts of pembrolizumab for 72 hrs. (22E) Pembrolizumab occupancy determined for T cells treated with increasing amounts of pembrolizumab for 72 hrs. N=4 patients. One-way ANOVA ***p<0.005. (22F) PD-1+/CD69+ T cells compared to T cells stimulated with spheroids ±100 μg/mL pembrolizumab. One-way ANOVA: **p<0.001. (22G) GEN26 spheroids were seeded ± matched T-cells and treated ±100 μg/mL pembrolizumab for 24 hrs. Spheroid viability determined by measuring RLU after 24 hrs. Six technical replicates. All error bars=SD.

FIGS. 23A-23B—Detection of T cell Infiltration and Analyte Secretion in Microtumor Model. (23A) Representative images of flow cytometric detection of PKH stained T cells following microtumor dissociation. Dual PKH and CD3 positivity was determined as a means to identify infiltrated T cells after seven days. (23B) Depicted are the concentrations of six tested analytes after day seven of culture for microtumors cultured alone or co-cultured with matched T cells. T cell response is more robust in the PD-L1 negative sample GEN19.

FIGS. 24A-24C—Microtumor model predicts response to Pembrolizumab. (24A) Microtumor growth rate was determined via PrestoBlue Viability assays from day 1 to day 7 for 100 μg/mL pembrolizumab and no treatment. Fold change was compared to no treatment. Mean of two replicates for four patients is depicted. (24B) Fold change analyte secretion was determined at day seven after 100 μg/mL pembrolizumab and compared to no treatment. Mean of two replicates for four patients is depicted. (24C) Pearson correlations were determined for pembrolizumab induced fold change in microtumor growth rate compared to pembrolizumab induced fold change in analyte secretion for TNFα, IFNγ, MIP-1α, MIP-1β.

FIGS. 25A-25H—Image based assay for measuring tumor and T-cell interactions for immuno-oncology (I/O) applications.

FIGS. 26A-H—T-cell conditioned media induced changes of immune cell composition of primary tumor tissue.

FIGS. 27A-C—Induced change in PD-L1 expression and detection of T-cell mediated apoptosis.

FIGS. 28A-C—Characterization of two patient samples of ovarian cancer from tissue resection through 3D spheroid culture. Specifically, FIG. 28A illustrates a schematic of tissue processing for characterization. Flow cytometry was conducted to evaluate cell composition following tissue dissociation and prior to 3D culture. Immunofluorescence and flow cytometry were conducted at different timepoints to evaluate 3D spheroid culture and drug treatment effects on cell populations. Representative images of formalin-fixed paraffin embedded tissues. Pan-cytokeratin was used to determine the presence of epithelial cells within the tumor tissues. Additional immune-related markers selected to assess infiltration were PD-L1, PD-1, CD8, and CD11c. Isotype controls in FIG. 28B were diluted to match marker dilutions. Representative images in FIG. 28C are from two independent experiments. Scare bar=75 μm.

FIGS. 29A-F—Gating strategy for tumor and immune cells. FIG. 29A illustrates representative data showing forward scatter (FSC) and side scatter (SSC) of OVC45 and OVC33 Pre 3D. Depicted are gates defined for tumor cells (tumor gate) and lymphocytes (lymphocyte gate). FIG. 29B shows the well-defined lymphocyte gate using smaller axes for FSC and SSC. FIG. 29C illustrates representative data showing dead cell exclusion using DRAQ7 versus event counts on a histogram plot for both tumor tissues. FIG. 29D illustrates representative data showing immune cell identification using CD45 as a marker. Histograms show live CD45 positive cells were found in both a large tumor cell gate and the smaller lymphocyte gate. FIG. 29E illustrates representative data of immune cells assessed using CD45 as a marker. CD45 positive cells were detected within the large gate defined for tumor cells. Quantification of PD-L1 negative immune cells and PD-L1 positive immune cells were identified Pre 3D and Post 3D for both tumor tissues is shown in the right panel. FIG. 29F illustrates representative data of immune cells assessed using CD45 as a marker within the lymphocyte gate. Quantification of PD-L1 negative immune cells and PD-L1 positive immune cells were identified Pre 3D and Post 3D for both patient samples is shown in the right panel. Two independent experiments and three independent experiments were conducted for Pre 3D and Post 3D, respectively. Post 3D for all data shown=48 hours in 3D culture. Isotype controls (black events) were subtracted from marker values (OVC45=blue events; OVC33=green events). Unpaired t tests were used for sample comparison using GraphPad. Error bars reflect SD. *p<0.05, **p<0.01.

FIGS. 30A-G—Determination of inter-patient proportion of T-cell subpopulations. FIG. 30A illustrates representative data and quantification of total EpCAM positivity within the tumor cell gate. FIG. 30B illustrates representative data and quantification of total CD3 positivity within the lymphocyte gate. Two independent experiments were conducted for Pre 3D and three independent experiments were conducted for Post 3D. Post 3D for all data shown=72 hours in culture. Isotype controls (black events) were subtracted from marker values (OVC45=blue events, OVC33=green events). Unpaired t tests were used for sample comparison using GraphPad. Error bars reflect SD. Not significant=n.s., *p<0.05, ***p<0.005. FIG. 30C illustrates representative data and quantification of CD4 positive T-cells (CD3+CD4+). FIG. 30D illustrates representative data and quantification of CD8 positive T-cells (CD3+CD8+). FIG. 30E illustrates representative images verify the presence of CD8 positive cells as determined by immunofluorescence following 48 hours in 3D spheroid culture. Scale bars=75 μm. Two independent experiments and three independent experiments were conducted for Pre 3D and Post 3D, respectively. FIG. 30F illustrates representative data of DCs defined by dual CD45 and CD11c positivity. The presence of DCs following 48 hours in 3D spheroid culture was determined via immunofluorescence. Scale bars=75 μm. Image inset shows a zoomed version of the same magnification. FIG. 30G illustrates representative data of DCs defined by dual CD45 and CD11c positivity. The percent of dual CD45 and CD11c events were determined for OVC45 and OVC33. Isotype controls (black events) were subtracted from marker values (OVC45=blue events, OVC33=green events). Expression of MHC-II and CD103 expression was determined for DC populations across the patient samples. Two independent experiments were conducted for OVC45 and three independent experiments were conducted for OVC33. Isotype controls were subtracted from marker values. Error bars reflect SD. Not significant=n.s., *p<0.05, **p<0.01, ***p<0.005.

FIGS. 31A-C—Impact of T-cells on PD-L1-EpCAM dual positive cells.

Specifically, FIG. 31A-C illustrates increased PD-L1+/EpCAM+ cell proportion observed Post 3D in OVC33 is partially T-cell dependent. FIG. 31A illustrates representative flow cytometry data of tumor cells assessed for PD-L1 expression using EpCAM cultured in 3D for 48 hours following T-cell depletion. FIG. 31B shows the quantification of the percent dual PD-L1/EpCAM positive cells following 48 hours of 3D culture with or without T-cell depletion.

FIG. 31C shows IFNγ secretion determined following 72 hours of 3D culture for bulk ovarian tumor cells with or without T-cell depletion.

FIGS. 32A-E—Characterization of T-cell populations for markers of activation. FIG. 32A illustrates representative data of CD4 positive T-cells evaluated for activation and exhaustion via PD-1 expression. FIG. 32B shows that Tregs were identified by dual CD4 and CD25 positivity. FIG. 32C illustrates that CD8 positive cytotoxic T-cells were examined by analyzing expression levels of PD-1. FIG. 32D illustrates that the activation status of CD8 positive cytotoxic T-cells was evaluated via CD69 expression. Two independent experiments were conducted for OVC45 and Pre 3D OVC33 and three independent experiments were conducted for OVC33 Post 3D. Post 3D for all data shown=72 hours in culture. Isotype controls (black events) were subtracted from marker values (OVC45=blue events, OVC33=green events). Unpaired t tests were used for sample comparison using GraphPad. FIG. 32E shows the determination of the concentration of cytokines from supernatants collected following 72 hours in 3D culture from three independent experiments for OVC45 and OVC33. Unpaired t tests were used for sample comparison using GraphPad. Error bars reflect SD. Not significant=n. s., *p<0.05, **p<0.01, ***p<0.005, ****p<0.001.

FIGS. 33A-C—Evaluation of different cell types and different activation mechanisms following a single treatment. FIG. 33A illustrates representative data and quantification of the activation status of cytotoxic T-cells was defined by dual CD8 high and CD69 high positivity following no treatment or T-cell CM treatment for 48 hours. Quantification of activated cytotoxic T-cells from three independent experiments for OVC45 and two independent experiments for OVC33. FIG. 33B shows the quantification of MHC-II high expression from two independent experiments. OVC45 and OVC33 is shown in blue or green, respectively. FIG. 33C illustrates representative data showing PD-L1 expression on tumor cells defined by dual PD-L1 and EpCAM positivity. Quantification of PD-L1 positive tumor cells from two independent experiments. Isotype controls (black events) were subtracted from marker values (OVC45=blue events, OVC33=green events). Unpaired t tests were used for sample comparison using GraphPad. Error bars reflect SD. *p<0.05, **p<0.01.

FIGS. 34A-D—Examination of changes in cytokine secretion and cell viability following treatment with pembrolizumab. FIG. 34A shows how cytokine secretion was determined from supernatants collected for both patient samples following VC or pembrolizumab treatment for 48 hours. Supernatant was collected from three independent experiments. Depicted is the fold change in cytokine concentrations as determined by normalizing pembrolizumab treatment to VC. FIG. 34B shows data from 3D spheroids that were treated with media (No Tx), VC, olaparib, pembrolizumab, or both olaparib and pembrolizumab (Combo) for 48 hours. FIG. 34C illustrates images of 3D spheroids following 48 hours of treatment. Scale bars=650 μm. FIG. 34D illustrates results from when Pre 3D bulk T-cells were separated and incubated with pembrolizumab. Pre 3D bulk cells were then either cultured with or without treated T-cells, and after 48 hours, spheroid viability was determined. Spheroid viability for all experiments was determined using CellTiter-Glo® Glo. Unpaired one-way ANOVA with multiple comparison was conducted on the mean of three independent experiments. Error bar=SD. Not significant=n.s., *p<0.05, **p<0.01.

FIGS. 35A-E—Evaluation of synergistic effect of treatment with durvalumab with olaparib. FIGS. 35A and 35B show the viability determination of OVC45 and OVC33 that were treated with a range of olaparib or durvalumab. Percent viability was determined by normalizing to VC. Dose-response curves and relative IC95 or absolute IC50 was determined across two independent experiments using Combenefit software. FIG. 35C depicts the mean and error of percent spheroid viability following olaparib and durvalumab cross dose-response treatment for OVC45 and OVC33 normalized to VC. FIG. 35D shows the Loewe synergy and antagonism that were determined from the change in spheroid viability following olaparib and durvalumab cross dose-response across two independent experiments. Synergistic combinations (blue) or antagonistic combinations (red) are only color coded if there is statistical significance. Synergy and antagonism heat maps and significance were calculated and generated by Combenefit software. * p<0.05. A single combination that is highlighted by the circled data set on the synergy heatmap for OVC45 and OVC33. FIG. 35E shows representative spheroid images of OVC33 are shown post treatment. Scale bar=650 μm.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +1-1% or less, and +1-0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide 3D in vitro tissue models and methods that maintain autologous patient tumor and immune cells for the testing and prediction of immune cell responses to treatments with various drugs. This type of modeling recapitulates the patient tumor microenvironment and allows for personalized methods of treatment which can include screening therapeutic agents and responsiveness of tumor cells to treatment modalities.

In specific embodiments of the methods of treating cancer, methods of measuring the level of immune cells in a 3D culture system, the 3D cancer cell models, and the 3D systems for co-culturing cells contemplated by the present invention, an original tissue sample is a tumor tissue sample. The tumor may include, without limitation, solid tumors such as sarcomas and carcinomas. Examples of solid tumors include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, epithelial carcinoma, bronchogenic carcinoma, hepatoma, colorectal cancer (e.g., colon cancer, rectal cancer), anal cancer, pancreatic cancer (e.g., pancreatic adenocarcinoma, islet cell carcinoma, neuroendocrine tumors), breast cancer (e.g., ductal carcinoma, lobular carcinoma, inflammatory breast cancer, clear cell carcinoma, mucinous carcinoma), ovarian carcinoma (e.g., ovarian epithelial carcinoma or surface epithelial-stromal tumour including serous tumour, endometrioid tumor and mucinous cystadenocarcinoma, sex-cord-stromal tumor), prostate cancer, liver and bile duct carcinoma (e.g., hepatocellular carcinoma, cholangiocarcinoma, hemangioma), choriocarcinoma, seminoma, embryonal carcinoma, kidney cancer (e.g., renal cell carcinoma, clear cell carcinoma, Wilm's tumor, nephroblastoma), cervical cancer, uterine cancer (e.g., endometrial adenocarcinoma, uterine papillary serous carcinoma, uterine clear-cell carcinoma, uterine sarcomas and leiomyosarcomas, mixed mullerian tumors), testicular cancer, germ cell tumor, lung cancer (e.g., lung adenocarcinoma, squamous cell carcinoma, large cell carcinoma, bronchioloalveolar carcinoma, non-small-cell carcinoma, small cell carcinoma, mesothelioma), bladder carcinoma, signet ring cell carcinoma, cancer of the head and neck (e.g., squamous cell carcinomas), esophageal carcinoma (e.g., esophageal adenocarcinoma), tumors of the brain (e.g., glioma, glioblastoma, medullablastoma, astrocytoma, medulloblastoma, craniopharyngioma, ependymoma, pinealoma, hemangioblastoma, acoustic neuroma, oligodenroglioma, schwannoma, meningioma), neuroblastoma, retinoblastoma, neuroendocrine tumor, melanoma, cancer of the stomach (e.g., stomach adenocarcinoma, gastrointestinal stromal tumor), or carcinoids. Lymphoproliferative disorders are also considered to be proliferative diseases.

In some embodiments of the methods of treating cancer, methods of measuring the level of immune cells in a 3D culture system, the 3D cancer cell models, and the 3D systems for co-culturing cells contemplated by the present invention, the ratio of tumor cells to immune cells may range anywhere from about 1:0.05 to about 1:100, such as from about 1:0.05 to about 1:20. The ratio may be about 1:0.05, 1:0.06, 1:0.07, 1:0.08, 1:0.09, 1:0.1, 1:0.2, 1:0.3, 1:0.4, 1:0.5, 1:0.6, 1:0.7, 1:0.8, 1:0.9, 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5, 1:10, 1:10.5, 1:11, 1:11.5, 1:12, 1:12.5, 1:13, 1:13.5, 1:14, 1:14.5, 1:15, 1:15.5, 1:16, 1:16.5, 1:17, 1:17.5, 1:18, 1:18.5, 1:19, 1:19.5, 1:20, 1:30; 1:40: 1:50, 1:60, 1:70, 1:80, 1:90, 1:100, or anywhere in between.

In some embodiments of the methods of treating cancer, methods of measuring the level of immune cells in a 3D culture system, the 3D cancer cell models, and the 3D systems for co-culturing cells contemplated by the present invention, a ratio of immune cells to tumor cells can be determined. In some embodiments, the ratio of tumor cells to immune cells is 1:0.05 to 1:100, such as 1:0.05 to 1:20. In specific embodiments, the ratio of tumor cells to immune cells is 1:10.

In some embodiments of the methods of treating cancer, methods of measuring the level of immune cells in a 3D culture system, the 3D cancer cell models, and the 3D systems for co-culturing cells contemplated by the present invention, the immune cells and the tumor cells are from the same subject.

In some embodiments of the methods of treating cancer, methods of measuring the level of immune cells in a 3D culture system, the 3D cancer cell models, and the 3D systems for co-culturing cells contemplated by the present invention, the immune cells comprise T cells, natural killer cells, dendritic cells, macrophages, or a combination thereof. In specific embodiments, the immune cells comprise T cells.

In some embodiments of the methods of treating cancer, methods of measuring the level of immune cells in a 3D culture system, the 3D cancer cell models, and the 3D systems for co-culturing cells contemplated by the present invention, at least one therapeutic agent can be utilized that comprises at least one checkpoint inhibitor. In some embodiments, the checkpoint inhibitor targets PD-1.

In some embodiments of the methods of treating cancer, methods of measuring the level of immune cells in a 3D culture system, the 3D cancer cell models, and the 3D systems for co-culturing cells contemplated by the present invention, the at least one therapeutic agent that is utilized further comprises at least one poly(ADP-ribose) polymerase inhibitor. In some embodiments, the at least one poly(ADP-ribose) polymerase inhibitor comprises olaparib, niraparib, rucaparib, talazoparib, or a combination thereof.

In some embodiments, the ratio of the checkpoint inhibitor to the PARP inhibitor can be from about 100:0 to about 0:100, such as from about 75:25 to about 25:75, such as about 50:50, or any ranges therebetween, depending on application and treatment.

In some embodiments of the methods of treating cancer, methods of measuring the level of immune cells in a 3D culture system, the 3D cancer cell models, and the 3D systems for co-culturing cells contemplated by the present invention, a cell mass can be formed that is a tumor spheroid.

In some embodiments of the methods of treating cancer, methods of measuring the level of immune cells in a 3D culture system, the 3D cancer cell models, and the 3D systems for co-culturing cells contemplated by the present invention, the responsiveness of the tumor cells is a decrease in viability.

In some embodiments of the methods of treating cancer, methods of measuring the level of immune cells in a 3D culture system, the 3D cancer cell models, and the 3D systems for co-culturing cells contemplated by the present invention, identifying a patient-specific treatment based on the decrease in tumor cell viability can be determined.

In some embodiments of the methods of treating cancer, methods of measuring the level of immune cells in a 3D culture system, the 3D cancer cell models, and the 3D systems for co-culturing cells contemplated by the present invention, the at least one therapeutic agent induces secretion of TNF-α, MIP-1α, and IFNγ.

In some embodiments of the methods of treating cancer, methods of measuring the level of immune cells in a 3D culture system, the 3D cancer cell models, and the 3D systems for co-culturing cells contemplated by the present invention, isolated immune cells can be used in cell therapy.

In another aspect, the methods of treating cancer, methods of measuring the level of immune cells in a 3D culture system, the 3D cancer cell models, and the 3D systems for co-culturing cells contemplated by the present invention invention can include use of a 3D cancer cell model comprising immune cells and tumor cells isolated from a subject that are co-cultured under conditions that allow the immune cells and the tumor cells to form a cell mass.

### Three-Dimensional Cancer Cell Models

In some embodiments, the invention provides a 3D cancer cell model comprising immune cells and tumor cells isolated from a subject that are co-cultured under conditions that allow the immune cells and the tumor cells to form a cell mass. The cancer cell model may be designed to comprise a particular ratio of immune cells and tumor cells, for example a ratio that is subject-specific.

As used herein, immune cells may include any cells of the innate or adaptive immune system, such as, but not necessarily limited to, monocytes, macrophages, dendritic cells, peripheral blood mononuclear cells, T cells, B cells, or natural killer cells, or a combination thereof.

In certain embodiments, a tissue sample is obtained from a subject. As used herein, a “sample”, “tissue sample”, or “biological sample” may contain whole cells and/or live cells and/or cell debris. The sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The tissue sample can be dissociated into a single cell population. Standard tissue dissociation techniques may be used. For example, tissue can be dissociated into single cell suspension using mechanical and enzymatic dissociation techniques. General techniques useful in the practice of this invention in cell culture and media uses are known in the art (e.g., Large Scale Mammalian Cell Culture (Hu et al. 1997. Curr Opin Biotechnol 8: 148); Serum-free Media (K. Kitano. 1991. Biotechnology 17: 73); or Large Scale Mammalian Cell Culture (Curr Opin Biotechnol 2: 375, 1991). The terms “culturing” or “cell culture” are common in the art and broadly refer to maintenance of cells and potentially expansion (proliferation, propagation) of cells in vitro. Typically, animal cells, such as mammalian cells, such as human cells, are cultured by exposing them to (i.e., contacting them with) a suitable cell culture medium in a vessel or container adequate for the purpose (e.g., a 96-, 24-, or 6-well plate, a T-25, T-75, T-150 or T-225 flask, or a cell factory), at art-known conditions conducive to in vitro cell culture, such as temperature of 37° C., 5% v/v CO₂ and >95% humidity. Furthermore, cells may be cultured for the expansion of cells like tumor cells or tumor-infiltrating lymphocytes (TILs), as described further below. Optionally, prior to carrying out further assays, the cells can be characterized for particular checkpoint target expression and/or CD8 or other markers.

In specific embodiments, an original tissue sample is a tumor tissue sample. The tumor may include, without limitation, solid tumors such as sarcomas and carcinomas. Examples of solid tumors include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, epithelial carcinoma, bronchogenic carcinoma, hepatoma, colorectal cancer (e.g., colon cancer, rectal cancer), anal cancer, pancreatic cancer (e.g., pancreatic adenocarcinoma, islet cell carcinoma, neuroendocrine tumors), breast cancer (e.g., ductal carcinoma, lobular carcinoma, inflammatory breast cancer, clear cell carcinoma, mucinous carcinoma), ovarian carcinoma (e.g., ovarian epithelial carcinoma or surface epithelial-stromal tumour including serous tumour, endometrioid tumor and mucinous cystadenocarcinoma, sex-cord-stromal tumor), prostate cancer, liver and bile duct carcinoma (e.g., hepatocellular carcinoma, cholangiocarcinoma, hemangioma), choriocarcinoma, seminoma, embryonal carcinoma, kidney cancer (e.g., renal cell carcinoma, clear cell carcinoma, Wilm's tumor, nephroblastoma), cervical cancer, uterine cancer (e.g., endometrial adenocarcinoma, uterine papillary serous carcinoma, uterine clear-cell carcinoma, uterine sarcomas and leiomyosarcomas, mixed mullerian tumors), testicular cancer, germ cell tumor, lung cancer (e.g., lung adenocarcinoma, squamous cell carcinoma, large cell carcinoma, bronchioloalveolar carcinoma, non-small-cell carcinoma, small cell carcinoma, mesothelioma), bladder carcinoma, signet ring cell carcinoma, cancer of the head and neck (e.g., squamous cell carcinomas), esophageal carcinoma (e.g., esophageal adenocarcinoma), tumors of the brain (e.g., glioma, glioblastoma, medullablastoma, astrocytoma, medulloblastoma, craniopharyngioma, ependymoma, pinealoma, hemangioblastoma, acoustic neuroma, oligodenroglioma, schwannoma, meningioma), neuroblastoma, retinoblastoma, neuroendocrine tumor, melanoma, cancer of the stomach (e.g., stomach adenocarcinoma, gastrointestinal stromal tumor), or carcinoids. Lymphoproliferative disorders are also considered to be proliferative diseases.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Co-culture conditions are envisioned that allow migration (e.g., the movement of cells from one compartment to another compartment) of immune cells to the tumor cells. In particular example embodiments, the tumor cells and the immune cells may form a cell mass, such as a tumor spheroid. As used herein, a cancer spheroid means a cell mass containing tumor initiating cells. Tumor progenitor cells mean cells that form tumors when transplanted into an immunodeficient animal. Cancer spheroids usually contain about 100 to about 10000, preferably about 500 to about 2000 cells, and are close to a sphere of about 0.01 mm to about 2 mm, preferably about 0.1 mm to about 0.5 mm in diameter. A spheroid is a sphere-like three dimensional shape, which can include prolate spheroids and oblate spheroids. Here, “diameter” means the length of the longest axis of cell mass.

In some embodiments, the ratio of tumor cells to immune cells may range anywhere from about 1:0.05 to about 1:100, such as from about 1:0.05 to about 1:20. The ratio may be about 1:0.05, 1:0.06, 1:0.07, 1:0.08, 1:0.09, 1:0.1, 1:0.2, 1:0.3, 1:0.4, 1:0.5, 1:0.6, 1:0.7, 1:0.8, 1:0.9, 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5, 1:10, 1:10.5, 1:11, 1:11.5, 1:12, 1:12.5, 1:13, 1:13.5, 1:14, 1:14.5, 1:15, 1:15.5, 1:16, 1:16.5, 1:17, 1:17.5, 1:18, 1:18.5, 1:19, 1:19.5, 1:20, 1:30; 1:40: 1:50, 1:60, 1:70, 1:80, 1:90, 1:100, or anywhere in between.

In specific embodiments, the ratio of tumor cells to immune cells is about 1:5 to about 1:15, such as about 1:8 to about 1:12, such as about 1:9 to about 1:11, or such as about 1:10. In embodiments, the ratio of tumor cells to immune cells is adjusted according to subject specific ratios, cancer-specific ratios, or tumor specific ratios.

In some embodiments, the immune cells and the tumor cells are from the same subject (autologous). In some embodiments, the immune cells and the tumor cells are from different subjects (allogeneic).

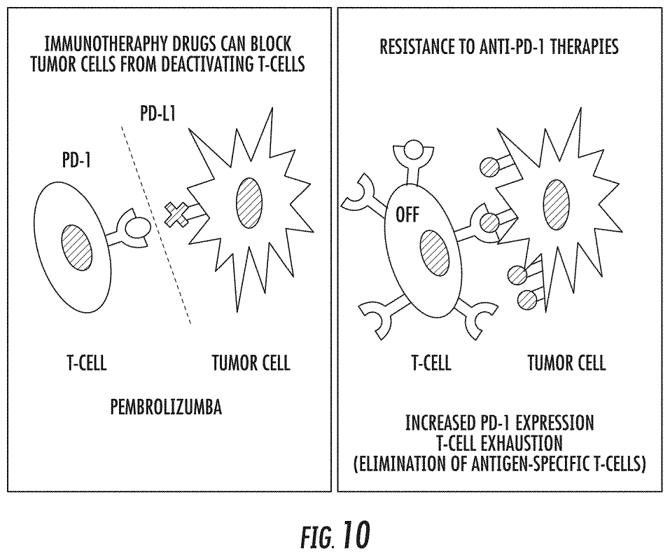

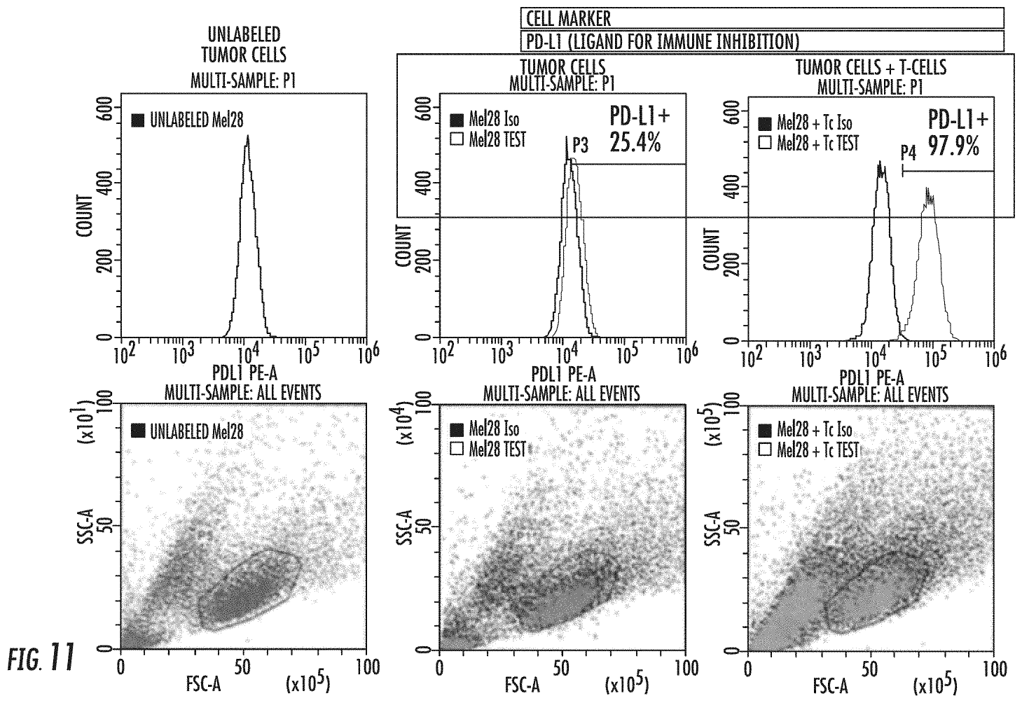

In some embodiments, the immune cells express an immune checkpoint protein, which may be targeted by at least one therapeutic agent. Immune checkpoints are inhibitory pathways that slow down or stop immune reactions and prevent excessive tissue damage from uncontrolled activity of immune cells. A model for possible adaptive immune resistance is shown in FIG. 10, while FIG. 11 summarizes data showing detection of adaptive immune resistance response by tumor cells when they are treated with T cells. For instance, PD-L1 on the cells increases in the presence of T cells. Specifically, when PD-1 is bound to PD-L1, it helps keep T cells from killing other cells, including cancer cells. As such, anticancer drugs, called immune checkpoint inhibitors, can be used to block PD-1. When this protein is blocked, the “brakes” on the immune system are released and the ability of T cells to kill cancer cells is increased.

In certain embodiments, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy? Biochem Soc Trans. 2016 Apr. 15; 44(2):356-62). SHP-1 is a widely expressed inhibitory protein tyrosine phosphatase (PTP). In T cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

WO2014172606 relates to the use of MT1 and/or MT2 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

Additional targets for immune checkpoints may include, but are not necessarily limited to, CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCY1A2, GUCY1A3, GUCY1B2, GUCY1B3, MT1, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT.

In some embodiments, the immune cells as described herein express an immune checkpoint protein such as CTLA4, BTLA, LAG3, ICOS, PD-1, PDL1, KIR, CD40, OX40, CD137, GITR, CD27 or TIM-3.

In specific embodiments, the immune checkpoint protein is PD-1.

In specific embodiments, the invention comprises use of at least one therapeutic agent. The at least one therapeutic agent can be a checkpoint inhibitor. Therapeutic agents may comprise a checkpoint inhibitor of one or more checkpoint proteins, and may be administered to the 3D systems disclosed herein to screen and identify potential modulating agents and/or treatments. Suitable checkpoint inhibitors bind to or inhibit any of the aforementioned checkpoint proteins. Suitable checkpoint inhibitors are well known in the art and include, but are not necessarily limited to, pembrolizumab, nivolumab, ipilimumab, anti-PVL1, durvalumab, atezolizumab, or a combination thereof.

In addition to a checkpoint inhibitor, the invention can also include the use of another therapeutic agent in combination with the checkpoint inhibitor. For instance, the invention can include at least one poly(ADP-ribose) polymerase inhibitor. Poly(ADP-ribose) polymerase inhibitors, which are often called PARP inhibitors, are targeted therapies that are used to treat cancer. PARP is a protein that has a role in cellular growth, regulation and cell repair which helps the cancer cells repair themselves and survive. The PARP inhibitor stops the cancer cells being repaired which causes the cells to die and so reduces tumor growth. In some embodiments, the at least one poly(ADP-ribose) polymerase inhibitor comprises olaparib, niraparib, rucaparib, talazoparib, or a combination thereof. The ratio of the checkpoint inhibitor to the PARP inhibitor can be from about 100:0 to about 0:100, such as from about 75:25 to about 25:75, such as about 50:50, or any ranges therebetween, depending on application and treatment.

### Three-Dimensional Systems for Co-Culturing Cells

In some embodiments, the invention provides a 3D system for co-culturing cells comprising a first compartment comprising immune cells isolated from a subject; a second compartment comprising tumor cells isolated from a subject; wherein the first and second compartments comprise a porosity that allows migration of immune cells between compartments; and wherein the first compartment and the second compartment are stacked relative to each other.

As used herein, a compartment may comprise any structure that initially maintains or separates a first cell type from a second cell type. In certain embodiments, a compartment may be a discrete volume or discrete space, such as a container, scaffold, receptacle, or other defined volume or space that can be defined by properties that prevent and/or inhibit migration of cells and reagents necessary to carry out the methods disclosed herein, for example a volume or space defined by physical properties such as walls, for example the walls of a well, tube, or a surface of a droplet, which may be impermeable or semipermeable, or as defined by other means such as chemical, diffusion rate limited, electro-magnetic, or light illumination, or any combination thereof. The term “diffusion rate limited” (for example diffusion defined volumes) refers to spaces are only accessible to certain molecules or reactions because diffusion constraints effectively defining a space or volume as would be the case for two parallel laminar streams where diffusion will limit the migration of a target molecule from one stream to the other. The term “chemical” defined volume or space refers to spaces where only certain target molecules can exist because of their chemical or molecular properties, such as size, where for example gel beads may exclude certain species from entering the beads but not others, such as by surface charge, matrix size or other physical property of the bead that can allow selection of species that may enter the interior of the bead. The term “electro-magnetically” defined volume or space refers to meant spaces where the electro-magnetic properties of the target molecules or their supports such as charge or magnetic properties can be used to define certain regions in a space such as capturing magnetic particles within a magnetic field or directly on magnets. The term “optically” defined volume refers to any region of space that may be defined by illuminating it with visible, ultraviolet, infrared, or other wavelengths of light such that only target molecules within the defined space or volume may be labeled. One advantage to the use of non-walled, or semipermeable compartments is that some reagents, such as buffers, chemical activators, or other agents maybe passed in and out through the discrete volume, while other material, such as target molecules, may be maintained in the discrete volume or space. Typically, a discrete volume will include a medium, (for example, an aqueous solution, an oil, a buffer, and/or a media capable of supporting cell growth) suitable for support of cell culture. Exemplary discrete volumes or spaces useful in the disclosed methods include droplets (for example, microfluidic droplets and/or emulsion droplets), hydrogel beads or other polymer structures (for example poly-ethylene glycol di-acrylate beads or agarose beads), tissue slides (for example, fixed formalin paraffin embedded tissue slides with particular regions, volumes, or spaces defined by chemical, optical, or physical means), microscope slides with regions defined by depositing reagents in ordered arrays or random patterns, tubes (such as, centrifuge tubes, microcentrifuge tubes, test tubes, cuvettes, conical tubes, and the like), bottles (such as glass bottles, plastic bottles, ceramic bottles, Erlenmeyer flasks, scintillation vials and the like), wells (such as wells in a plate), plates, pipettes, or pipette tips among others. In certain example embodiments, the individual discrete volumes are the wells of a microplate. In certain example embodiments, the microplate is a 96 well, a 384 well, or a 1536 well microplate. In some embodiments, the compartment may comprise a compartment as disclosed in U.S. Pat. No. 8,865,460, US Patent Application Publication No. 2012/0183987, or US Patent Application Publication No. 2015/0247112, all of which are incorporated by reference herein.

In certain example embodiments, each compartment may be connected by a means that allows for migration of cells between compartments. In other embodiments, a compartment may be a structure like a cell scaffold material, such as a disk or cube scaffold. In specific embodiments, the scaffold is a porous scaffold, suitable for tissue engineering. Another object of the present invention is to provide a porous scaffold obtainable by the above method, and its use in tissue engineering, cell culture and cell transport.

A schematic of one possible embodiment for a 3D microtumor is shown in FIG. 14. Immune cells and tumor cells can be placed in two separate compartments that are stacked relative to each other (left). Tumor cells then recruit immune cells to form a cell mass (right).

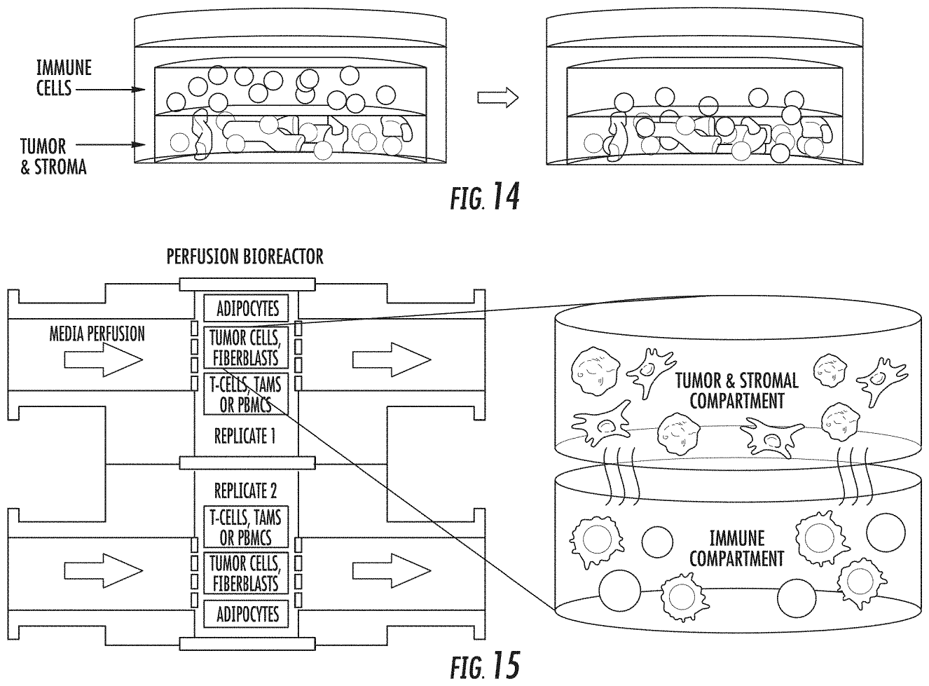

Meanwhile, FIG. 15 is a schematic of a 3D microtumor similar to FIG. 14, except that the microtumor is shown inside a perfusion bioreactor. In certain embodiments, the bioreactor could also be static. Co-culture of immune and tumor cells could be direct or segregated in various embodiments, and culture could progress over 10 days to 64 days. Tissue engineering is generally defined as seeding cells on or within a scaffold suitable for transplantation to produce an equivalent of a tissue or organ. The scaffold must be biocompatible and the cells must be able to attach and proliferate on the scaffold to form the equivalent of a tissue or organ. Thus, the scaffold can be considered as a substrate for cell growth in vitro or in vivo.

Ideal biocompatibility scaffold properties include the ability to support cell growth in vitro or in vivo, the ability to support the growth of a wide range of cell types or lineages, the ability to have varying levels of flexibility or stiffness required, and varying levels of biodegradability, the ability to be introduced into the desired site in vivo without causing secondary damage, and the ability to serve as a reservoir or carrier for the delivery of the drug or bioactive material to the desired site of action.

Many different scaffold materials have been used for induced tissue regeneration and/or as biocompatible surfaces. Biodegradable polymeric materials are preferred in many cases, because scaffolds degrade over time and the cell-scaffold structure is wholly replaced by cells. Among the many candidates that can serve as useful scaffolds needed to support tissue growth or regeneration include gels, foams, sheets, and multiple porous particle structures of different shapes and shapes.

In certain example embodiments the first and second scaffold may reside within a common chamber. In such embodiments, a physical means connecting the first and second scaffold is not required as cells may migrate directly between the first and second scaffold by means of a common medium or within the shared chamber.

In one embodiment, the present invention is directed to multi-chambered co-culture systems. The systems of the invention can be utilized for the growth and development of isolated cells of one or more cell types in a dynamic in vitro environment more closely resembling that found in vivo. For instance, a multi-chambered or multi-compartmental system can allow biochemical communication between cells of different types while maintaining the different cell types in a physically separated state, and moreover, can do so while allowing the cell types held in any one chamber to grow and develop with a three-dimensional aspect. In addition, the presently disclosed devices and systems can allow for variation and independent control of environmental factors within the individual chambers. For instance, the chemical make-up of a nutrient medium that can flow through a chamber as well as the mechanical force environment within the chamber including the perfusion flow, hydrostatic pressure, and the like, can be independently controlled and maintained for each separate culture chamber of the disclosed systems.

In some embodiments, the compartment may be porous to the extent that it allows migration of immune cells between two or more compartments. There are some systems in which an attempt has been made to physically separate cell types in dynamic systems, for instance through location of a porous substrate between the two cell types. However, in these systems, all cell-types cultured in the system are still subjected to the same culture media, similar to the above-described static systems. Additionally, the porous substrate usually also serves as the support scaffold to which cells are intended to attach and grow. Attachment of cells to the porous substrate will alter the flow characteristics of biochemicals across and through the substrate, which in turn affects communication between the cells.

In certain example embodiments, the first compartment and the second compartment are stacked relative to each other. In some embodiments, the compartment comprising the immune cells is stacked on top of the compartment comprising the tumor cells. In other embodiments, the compartment comprising the tumor cells is stacked on top of the compartment comprising the immune cells.

In one application the co-culture systems of the invention can be utilized for culturing product cells for medical use, for instance, for transplant to a patient or for manufacture of a protein product, such as a biopharmaceutical. According to this embodiment, cells can be grown in an environment that includes the biochemical products of different cell types, at least some of which may be necessary for the growth and development of the desired cells. However, cell types can be maintained in a physically isolated state during their growth and development. As such, possible negative consequences due to the presence of aberrant or undesired cell types in the desired product cells can be avoided.

It should be noted that the patient being treated has the disease (e.g., cancer), not the cells, and the focus of the present application is on outcome driven metrics in the patient once the therapeutic has been administered. Data analytics and high success rates are some of the powerful and unique features of the technology that differ over the prior art.

In some embodiments, cells may be clonally expanded prior to being placed or seeded in a compartment. Such methods are described in U.S. Pat. No. 8,637,307, which is herein incorporated by reference in its entirety. For example, the number of T cells and/or tumor cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000 fold, or most preferably at least about 100,000-fold. The numbers of T or tumor cells may be expanded using any suitable method known in the art. Exemplary methods of expanding the numbers of cells are described in patent publication No. WO 2003057171, U.S. Pat. No. 8,034,334, and U.S. Patent Application Publication No. 2012/0244133, each of which is incorporated herein by reference.

In some embodiments, the method further comprises determining a ratio of immune cells to tumor cells prior to generating the 3D cancer cell model. In some embodiments, the ratio of tumor cells to immune cells is 1:0.05 to 1:100, such as 1:0.05 to 1:20. In specific embodiments, the ratio of tumor cells to immune cells is 1:10.

In some embodiments, the immune cells and the tumor cells are from the same subject.

In some embodiments, the immune cells comprise T cells, natural killer cells, dendritic cells, macrophages, or a combination thereof. In specific embodiments, the immune cells comprise T cells.

In some embodiments, the at least one therapeutic agent comprises at least one checkpoint inhibitor. In some embodiments, the checkpoint inhibitor targets PD-1.

In some embodiments, the at least one therapeutic agent further comprises at least one poly(ADP-ribose) polymerase inhibitor. In some embodiments, the at least one poly(ADP-ribose) polymerase inhibitor comprises olaparib, niraparib, rucaparib, talazoparib, or a combination thereof.

In some embodiments, the ratio of the checkpoint inhibitor to the PARP inhibitor can be from about 100:0 to about 0:100, such as from about 75:25 to about 25:75, such as about 50:50, or any ranges therebetween, depending on application and treatment.

In some embodiments, the cell mass is a tumor spheroid.

In some embodiments, the responsiveness of the tumor cells is a decrease in viability.

In some embodiments, the method further comprises identifying a patient-specific treatment based on the decrease in tumor cell viability.

In some embodiments, the at least one therapeutic agent induces secretion of TNF-α, MIP-1α, and IFNγ.

In some embodiments, the method further comprises using isolated immune cells for use in a cell therapy.

In another aspect, the invention comprises a 3D cancer cell model comprising immune cells and tumor cells isolated from a subject that are co-cultured under conditions that allow the immune cells and the tumor cells to form a cell mass.

In one embodiment, ex vivo expansion of cells can be performed by isolation of cells and subsequent stimulation or activation followed by further expansion. In one embodiment of the invention, the cells may be stimulated or activated by a single agent. In another embodiment, cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment both primary and secondary agents are co-immobilized on a surface, for example a bead or a cell. In one embodiment, the molecule providing the primary activation signal may be a CD3 ligand on a T cell, and the co-stimulatory molecule may be a CD28 ligand or 4-1BB ligand.

Cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells and/or tumor cells can be expanded in vitro or in vivo.

In specific embodiments, the immune cells are clonally expanded prior to being placed in the first compartment. In specific embodiments, the tumor cells are clonally expanded prior to being placed in the second compartment. In some embodiments, both the immune cells and the tumor cells are clonally expanded prior to being placed in the respective compartments.

In some embodiments, the ratio of tumor cells to immune cells may range anywhere from about 1:0.05 to about 1:100, such as from about 1:0.05 to about 1:20. The ratio may be about 1:0.1, 1:0.2, 1:0.3, 1:0.4, 1:0.5, 1:0.6, 1:0.7, 1:0.8, 1:0.9, 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5, 1:10, 1:10.5, 1:11, 1:11.5, 1:12, 1:12.5, 1:13, 1:13.5, 1:14, 1:14.5, 1:15, 1:15.5, 1:16, 1:16.5, 1:17, 1:17.5, 1:18, 1:18.5, 1:19, 1:19.5, 1:20, 1:30; 1:40: 1:50, 1:60, 1:70, 1:80, 1:90, 1:100, or anywhere in between.

In specific embodiments, the ratio of tumor cells to immune cells is 1:10.

In some embodiments, the immune cells and the tumor cells are from the same subject or autologous. In some embodiments, the immune cells and tumor cells are from different subjects or allogeneic. In some embodiments, the immune cells are isolated from a healthy subject. In some embodiments, the immune cells express an immune checkpoint protein, as described earlier. In some embodiments, the immune cells as described herein express an immune checkpoint protein such as CTLA4, BTLA, LAG3, ICOS, PD-1, PD-L1, KIR, CD40, OX40, CD137, GITR, CD27 or TIM-3. In specific embodiments, the immune cells express PD-1.

### Methods of Screening Therapeutic Agents for Treating Cancer

In some embodiments, the invention comprises a method of screening one or more therapeutic agents for to administer to a patient for the treatment of cancer comprising co-culturing immune cells and tumor cells isolated from a subject under conditions that allow the immune cells and the tumor cells to form a cell mass, exposing the cell mass to at least one therapeutic agent, and measuring the responsiveness of the tumor cells in the cell mass to the at least one therapeutic agent.

It is to be understood that the term “treatment” as used above refers to the delivery of a therapeutic agent that provides some beneficial effect upon administration to a patent. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

The compositions and methods described herein comprise exposing the cell mass to one or more therapeutic agents. Embodiments may comprise exposing to a single agent or a combination of multiple agents, for example two, three, four, five, six or more agents. Exposing the agents may comprise administering multiple agents together, separately, or over different time courses. The ex vivo model system derived from a subject to be treated and the agents screened are to select for the best treatment or treatment combination. Accordingly, a variety of permutations of single or multiple agents administered, time course of exposing the cell-based system, dose of agents and varying combinations of agents can be utilized to optimize selection of treatment.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

Preferably, the therapeutic agent may be administered in a therapeutically effective amount of the active components. The term “therapeutically effective amount” refers to an amount which can elicit a biological or medicinal response in a tissue, system, animal or human that is being sought by a researcher, veterinarian, medical doctor or other clinician, and in particular can prevent or alleviate one or more of the local or systemic symptoms or features of a disease or condition being treated.

For example, in methods for treating cancer in a subject, an effective amount of a combination of inhibitors targeting epigenetic genes is any amount that provides an anti-cancer effect, such as reduces or prevents proliferation of a cancer cell or is cytotoxic towards a cancer cell.

In some embodiments, the method may further comprise determining a ratio of immune cells to tumor cells prior to generating the 3D cancer cell model.

In some embodiments, the ratio of tumor cells to immune cells ranges from about 1:0.05 to about 1:100, such as from about 1:0.05 to about 1:20, as described elsewhere herein. In specific embodiments, the ratio of tumor cells to immune cells is about 1:10.

In some embodiments, the immune cells and the tumor cells are from the same subject, as described elsewhere herein.

In some embodiments, the immune cells comprise T cells, natural killer cells, dendritic cells, macrophages, or a combination thereof, as described elsewhere herein. In specific embodiments, the immune cells are T cells.

In some embodiments, the at least one therapeutic agent comprises at least one checkpoint inhibitor. In specific embodiments, the checkpoint inhibitor targets PD-1.

As described elsewhere herein, drugs that target these checkpoint proteins are well known in the art and include, but are not necessarily limited to, pembrolizumab, nivolumab, ipilimumab, anti-PVL1, durvalumab, atezolizumab, or a combination thereof.

In addition to a checkpoint inhibitor, the invention can also include the use of another therapeutic agent in combination with the checkpoint inhibitor. For instance, the invention can include at least one poly(ADP-ribose) polymerase inhibitor. Poly(ADP-ribose) polymerase inhibitors, which are often called PARP inhibitors, are targeted therapies that are used to treat cancer. PARP is a protein that has a role in cellular growth, regulation and cell repair which helps the cancer cells repair themselves and survive. The PARP inhibitor stops the cancer cells being repaired which causes the cells to die and so reduces tumor growth. In some embodiments, the at least one poly(ADP-ribose) polymerase inhibitor comprises olaparib, niraparib, rucaparib, talazoparib, or a combination thereof. Further, the ratio of the checkpoint inhibitor to the PARP inhibitor can be from about 100:0 to about 0:100, such as from about 75:25 to about 25:75, such as about 50:50, or any ranges therebetween, depending on application and treatment.

In specific embodiments, the cell mass is a tumor spheroid, as described earlier. The method may further comprise measuring responsiveness of the tumor cells in the cell mass to the at least one therapeutic agent. Responsiveness may be measured in a number of ways. In some embodiments, the responsive phenotype is measured by a change in one or more cell types or cell states of the cell mass or spheroid. The change in one or more cell types of cell states of the cell mass or spheroid can, in embodiments, indicate reduced fitness of the cell mass or cell death of one or more target cell types in the cell mass.

In some embodiments, the non-responsive phenotype is measured by no change in cell mass phenotype or a change in one or more cell types or cell states indicating increased growth or fitness of the cell mass or one or more cell types in the cell mass.

In some embodiments, responsiveness of the tumor cells is a decrease in viability or cell death.

In some embodiments, the method may further comprise identifying a patient-specific treatment based on the decrease in tumor cell viability.

In some embodiments, secretion of analytes from immune cells may be correlated with decreased microtumor growth rate. For example, in some embodiments, a therapeutic agent may induce secretion of analytes from immune cells, which correlates with death or lack of viability of tumor cells, as described in Example 6. In specific embodiments, the therapeutic agent induces secretion of TNF-α and MIP-1α by T cells. In some embodiments, the therapeutic agent may induce secretion of IFNγ and MIP-1β.

In some embodiments, the method may further comprise isolating immune cells from the formed cell mass and further expanding the immune cells as described elsewhere herein, for use in a cell therapy.

Cell therapy is therapy in which cellular material is injected, grafted or implanted into a patient; this generally means intact, living cells. For example, T cells capable of fighting cancer cells via cell-mediated immunity may be injected in the course of immunotherapy.

Cell therapy may include allogeneic cell therapy, where the donor is a different person to the recipient of the cells, or autologous cell therapy, where the donor cells are from the patient in need of therapy. In some embodiments, the cell therapy may comprise adoptive cell therapy. As used herein, “ACT”, “adoptive cell therapy” and “adoptive cell transfer” may be used interchangeably. In certain embodiments, adoptive cell therapy (ACT) can refer to the transfer of cells to a patient with the goal of transferring the functionality and characteristics into the new host by engraftment of the cells (see, e.g., Mettananda et al., Editing an α-globin enhancer in primary human hematopoietic stem cells as a treatment for β-thalassemia, Nat Commun. 2017 Sep. 4; 8(1):424). As used herein, the term “engraft” or “engraftment” refers to the process of cell incorporation into a tissue of interest in vivo through contact with existing cells of the tissue. Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing graft-versus-host disease (GVHD) issues. The adoptive transfer of autologous tumor infiltrating lymphocytes (TIL) (Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57.) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma and colorectal carcinoma, as well as patients with CD19-expressing hematologic malignancies (Kalos et al., (2011) Science Translational Medicine 3 (95): 95ra73). In certain embodiments, allogenic cells immune cells are transferred (see, e.g., Ren et al., (2017) Clin Cancer Res 23 (9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

### Methods of Measuring the Level of Migration of Immune Cells

In some embodiments, the invention comprises a method of measuring the level of migration of immune cells in a 3D co-culture system (e.g., the movement of cells from one compartment to another compartment) comprising seeding immune cells isolated from a subject in a first compartment; seeding tumor cells isolated from a subject in a second compartment; culturing the immune cells and the tumor cells under conditions that allow migration of the immune cells from the first compartment to the second compartment; exposing at least the second compartment to at least one therapeutic agent; and measuring the responsiveness of the tumor cells to the at least one therapeutic agent.

In some embodiments, the compartment comprising the immune cells is stacked on top of the compartment comprising the tumor cells. In some embodiments, the compartment comprising the tumor cells is stacked on top of the compartment comprising the immune cells.

In some embodiments, migration of the immune cells from the first compartment to the second compartment allows the immune cells to form a cell mass with the tumor cells, as described earlier.

In some embodiments, the isolated immune cells are clonally expanded prior to seeding them in the first compartment. In some embodiments, the isolated tumor cells are clonally expanded prior to seeding them in the second compartment. In some embodiments, both the isolated immune cells and the isolated tumor cells are clonally expanded prior to seeding them in the first and second compartments, as described earlier.

In some embodiments, the ratio of tumor cells to immune cells ranges from about 1:0.05 to about 1:100, such as from about 1:0.05 to about 1:20. In some embodiments, the ratio of tumor cells to immune cells is about 1:10.

In some embodiments, the immune cells and the tumor cells are from the same subject. In some embodiments, the immune cells and the tumor cells are from different subjects.

In some embodiments, the immune cells express an immune checkpoint protein such as CTLA4, BTLA, LAG3, ICOS, PD-1, PDL1, KIR, CD40, OX40, CD137, GITR, CD27, TIM-3, or any other checkpoint proteins described above.

In some embodiments, the at least one therapeutic agent is an immune checkpoint inhibitor. In specific embodiments, the immune checkpoint inhibitor targets PD-1.

In some embodiments, the immune checkpoint inhibitor may include, but is not necessarily limited to, pembrolizumab, nivolumab, ipilimumab, anti-PVL1, durvalumab, atezolizumab, or a combination thereof.

In specific embodiments, the immune checkpoint inhibitor is pembrolizumab.

In addition to a checkpoint inhibitor, the invention can also include the use of another therapeutic agent in combination with the immune checkpoint inhibitor. For instance, the invention can include at least one poly(ADP-ribose) polymerase inhibitor. Poly(ADP-ribose) polymerase inhibitors, which are often called PARP inhibitors, are targeted therapies that are used to treat cancer. PARP is a protein that has a role in cellular growth, regulation and cell repair which helps the cancer cells repair themselves and survive. The PARP inhibitor stops the cancer cells being repaired which causes the cells to die and so reduces tumor growth. In some embodiments, the at least one poly(ADP-ribose) polymerase inhibitor comprises olaparib, niraparib, rucaparib, talazoparib, or a combination thereof.

In some embodiments, the ratio of the checkpoint inhibitor to the PARP inhibitor can be from about 100:0 to about 0:100, such as from about 75:25 to about 25:75, such as about 50:50, or any ranges therebetween, depending on application and treatment.

In some embodiments, the cells in the second compartment are a cancer spheroid, as described elsewhere herein.

In some embodiments, the responsiveness comprises death of the tumor cells, as described elsewhere herein.

The invention is further described in the following examples, which do not limit the scope of the invention described in the claims.

### EXAMPLES

### Example 1—Cytotoxic T Cell Mediated Tumor Spheroid Death

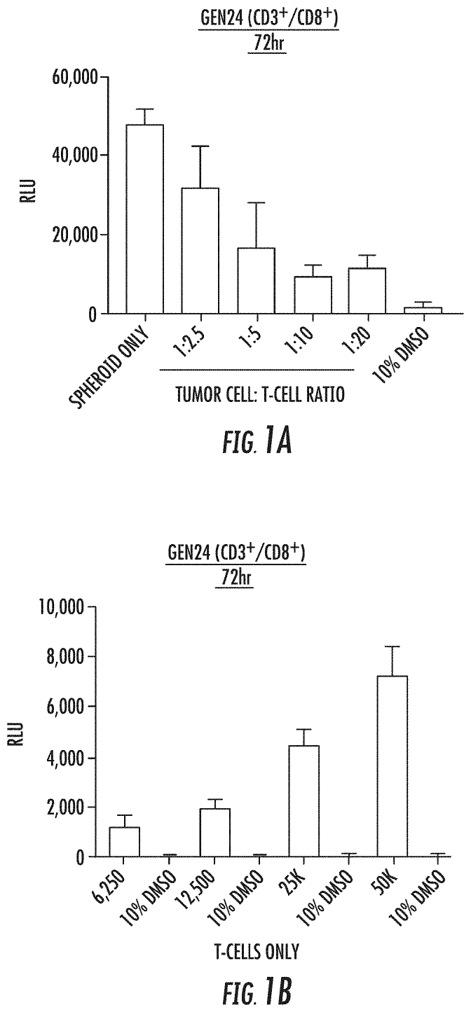

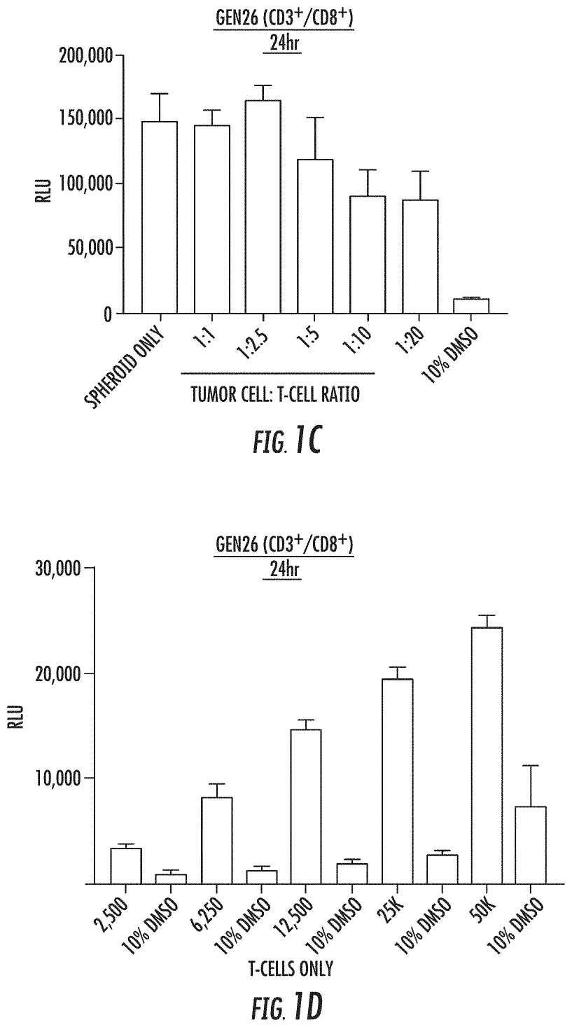

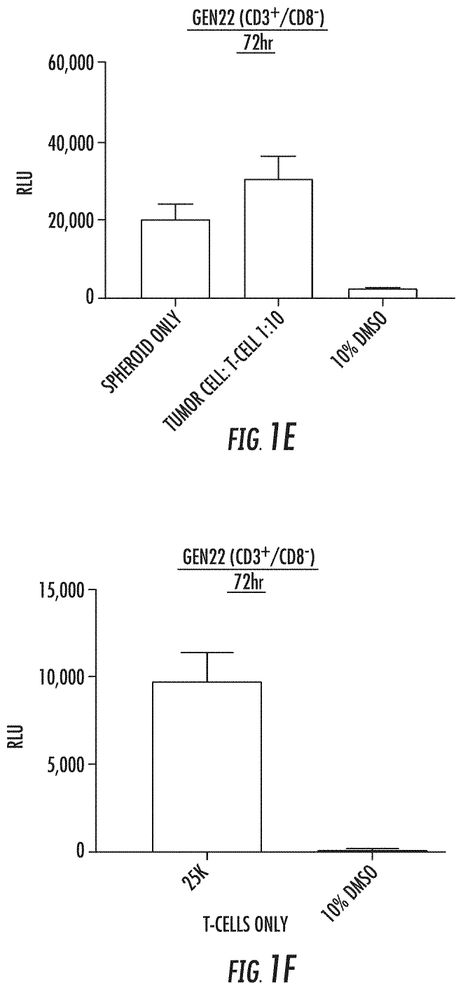

Resected melanoma tumors were dissociated and cultured for the expansion of tumor cells and autologous CD3+ Tumor-infiltrating lymphocytes (TILs). Prior to initiating tumor spheroid assays, expanded tumor cells and CD3+ TILs were characterized for PD-L1 expression or CD8 positivity, respectively (Tables 1 and 2). Applicant first sought to determine if the expanded T cells possess the capacity to kill matched tumor spheroids. Applicant utilized matched tumor cells and T cells from patient GEN24, which has a CD3+/CD8+ TIL population, to identify a lethal tumor cell per spheroid to T cell ratio. At increasing ratios, Applicant detected a step-wise decrease in tumor spheroid viability (FIG. 1A). This result suggests this decrease in viability is directly related to an increase in tumor spheroid death since T cells alone were found to be viable at all T cell numbers tested (FIG. 1B). A cell death control, 10% DMSO, was used for comparison. Applicant found that cytotoxic T cell-induced tumor spheroid death plateaued at about a 1:10 ratio. Next, Applicant compared these results to patient GEN26, which harbors CD3+/CD8+ TILs, but Applicant shortened the co-culture time from 72 hours to 24 hours. Even at this shorter timepoint, Applicant detected a decrease in tumor spheroid viability which again plateaued at about 1:10 ratio in the presence of viable T cells (FIG. 1C, 1D). To test that the measured cytotoxic T cell mediated tumor spheroid death is in fact dependent on the presence of a CD3+/CD8+ population, Applicant utilized TILs from patient GEN22 which were characterized as possessing no detectable CD3+/CD8+ population (Table 1). At the lethal ratio of about 1:10, Applicant did not observe a decrease in matched tumor spheroid viability (FIG. 1E). In fact, Applicant detected an increase in viability compared to tumor spheroid alone which is likely due to the presence of co-cultured viable T cells (FIG. 1F). These results demonstrate that CD8 specific cytotoxic T cell function can be monitored through autologous tumor spheroid death.

TABLE 1
Characteristics of Resected Melanoma Samples. Seven patient
samples were utilized for this study. Patient ID, metastatic site,
and current clinical treatments are depicted. Following expansion
of tumor cells and CO3+ TILs, samples were evaluated for the
presence of PD-L1 expression or CD8 positivity, respectively.

| Patient | Metastatic | | PD-L1+ | co3+/cos+ |
|---|---|---|---|---|
| ID | site | Treatment | Melanoma | TILs |
| GEN19 | Vulva | Pembrolizumab | No | 82% |
| GEN20 | Brain | Nivolumab | Yes | Not Tested |
| GEN21 | Lung | Ipilimumab | Yes | 95% |
| GEN22 | Brain | Pembrolizumab | Not Tested | 0% |
| GEN24 | Brain | No Treatment | Yes | 74% |
| GEN25 | Brain | Radiation | No | 63% |
| GEN26 | Lymph node | Ipilimumab | Yes | 86% |

TABLE 2
Characteristics of Resected Melanoma Samples.

| | | | | | CD3+/ |
|---|---|---|---|---|---|
| Patient | Metastatic | | CD44+ | PD-L1+ | CD8+ |
| ID | site | Treatment | Melanoma | Melanoma | TILs |
| GEN19 | Groin | Pembrolizumab | 93% | 0% | 82% |
| GEN20 | Vulva | Nivolumab | 99% | 45% | 73% |
| GEN21 | Lung | Ipilimumab | 90% | 11% | 95% |
| GEN26 | Lymph node | Ipilimumab | 99% | 6% | 86% |

### Example 2—Detection of Tumor Spheroid-Specific T Cell Responses in a Personalized Manner

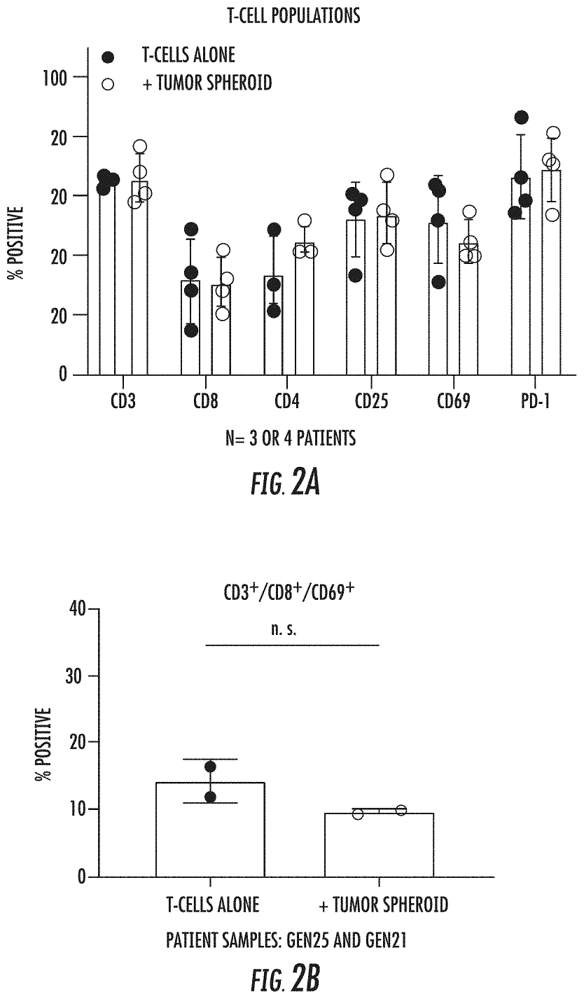

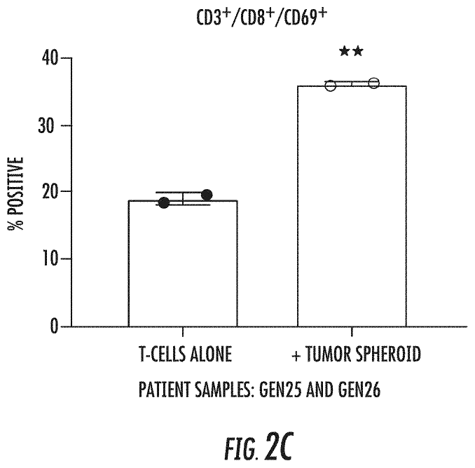

Applicant next wanted to determine the effects of tumor spheroids on autologous T cell populations across multiple patients. The overall number of CD3+ TILs did not significantly change following stimulation with tumor spheroids (FIG. 2A). Applicant detected a modest shift in CD4+ populations which increased across all patient samples tested, although not significantly. Overall, Applicant was unable to detect substantial changes in T cell populations following co-culture with matched tumor spheroids when all patients were evaluated together. However, T cell population shifts differed across patient samples, and Applicant therefore concluded that results should be evaluated at the individual level. For instance, GEN19 and GEN26 T cells displayed an increase in CD8+/CD69+ positive CD3+ subtypes unlike GEN21 and GEN25 which remained unchanged when stimulated with matched tumor spheroids (FIGS. 2B and 2C). With this perspective, Applicant was able to establish a platform that can induce and analyze T-cell population shifts in a personalized manner which may be reflective of the intrinsic tumor reactivity of the patient's TILs.

### Example 3—Detection of Pembrolizumab Occupancy and Pembrolizumab Induced Tumor Spheroid Death

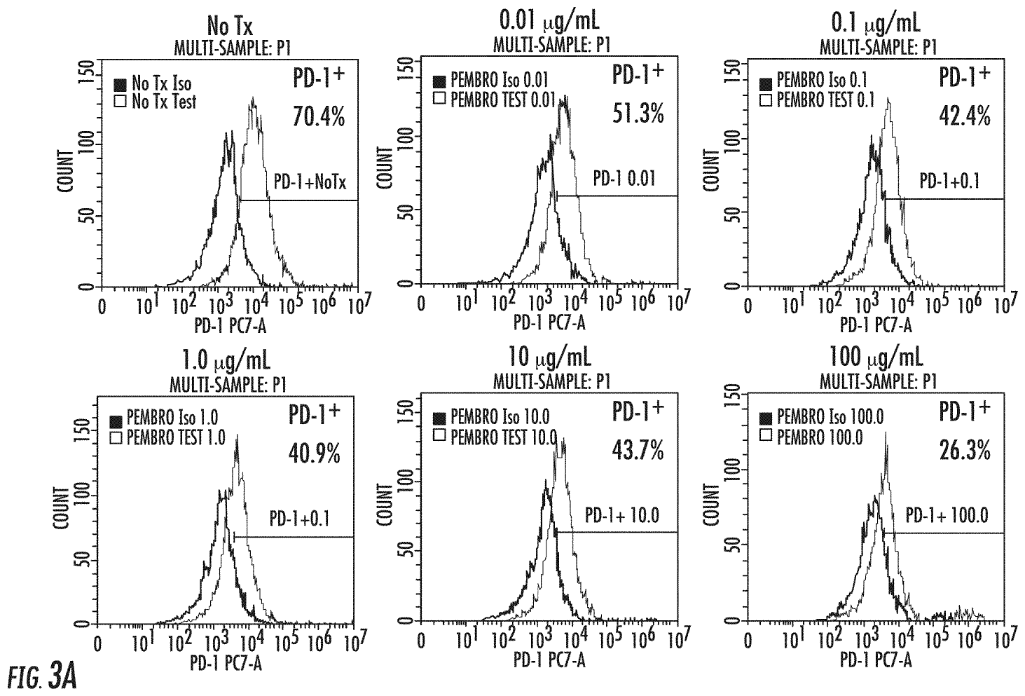

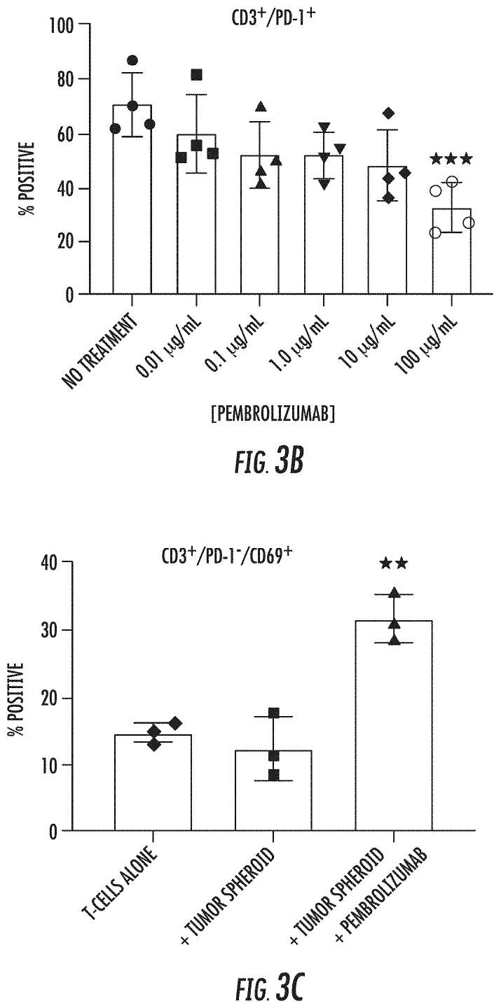

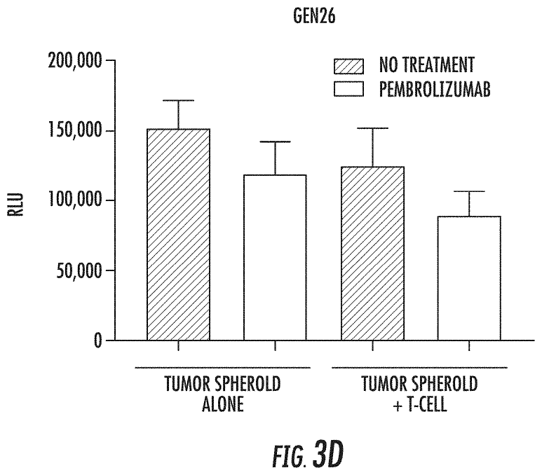

To evaluate potential pembrolizumab-mediated modulation of T cell responses when co-cultured with matched tumor spheroids, Applicant first determined if pembrolizumab binding to T cells could be detected. Representative flow cytometric data shows a right-ward shift in spectra in a dose dependent manner which is indicative of pembrolizumab binding to its target PD-1 protein (FIG. 3A). PD-1 occupancy was determined for four patients at increasing amounts of pembrolizumab. Applicant detected a downward trend in PD-1 site availability at increasing amounts of pembrolizumab with the highest concentration being significantly lower compared to no treatment (FIG. 3B). Applicant therefore used this concentration of pembrolizumab for all downstream experiments. Applicant then tested if pembrolizumab could increase T cell activation in the presence of matched tumor spheroids at the patient specific level. Of the four patient samples tested, three demonstrated a significant increase in CD3+/PD-1−/CD69+ levels in the presence on tumor spheroids and pembrolizumab compared to T cells with or without tumor spheroid stimulation (FIG. 3C). GEN25 did not display an increase in the CD3+/PD-1−/CD69+ population (data not shown). Of the three patients which demonstrated a pembrolizumab induced increase in T cell activation, Applicant was able to test GEN26 to determine if pembrolizumab could enhance T cell mediated tumor spheroid death. Applicant chose a tumor cell to T cell ratio of 1:5, which did not result in maximum tumor spheroid death at a 24 hour time point (FIG. 1C). A slight decrease in viability was detected for tumor spheroids alone when treated with pembrolizumab compared to no treatment. This result was comparable to tumor spheroid viability when co-cultured with cytotoxic T cells. The greatest reduction in tumor spheroid viability was observed when they were co-cultured with T cells and treated with pembrolizumab (FIG. 3D). This result demonstrates the tumor spheroid assay can be utilized to identify patient specific pembrolizumab responses via increase in T cell activation and decrease in tumor spheroid viability.

### Example 4—Detection of T Cell Infiltration in a Microtumor Model

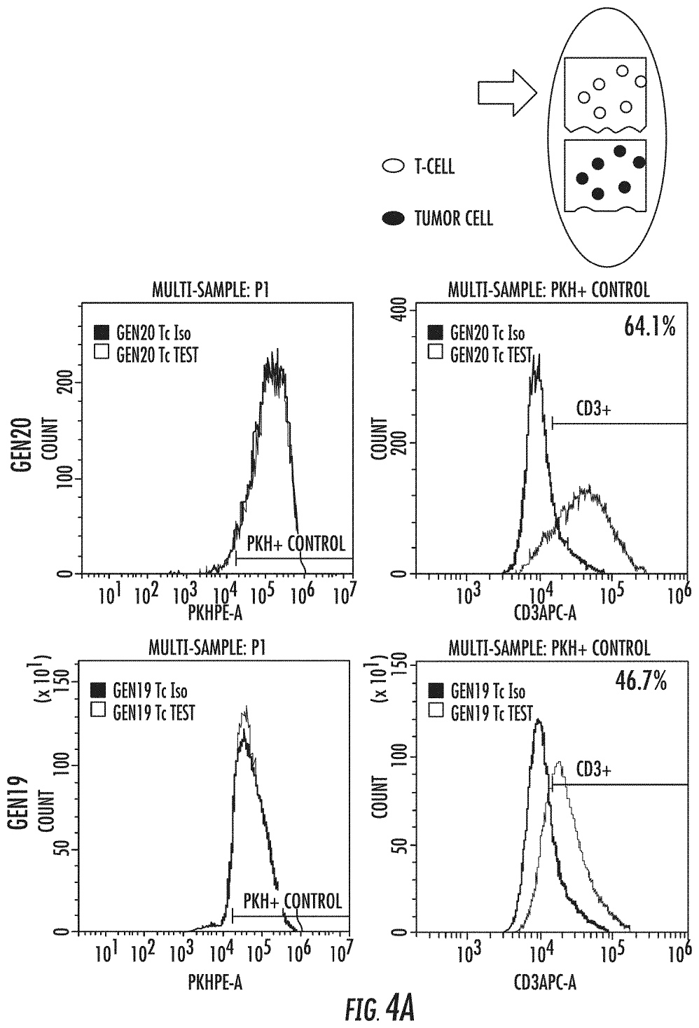

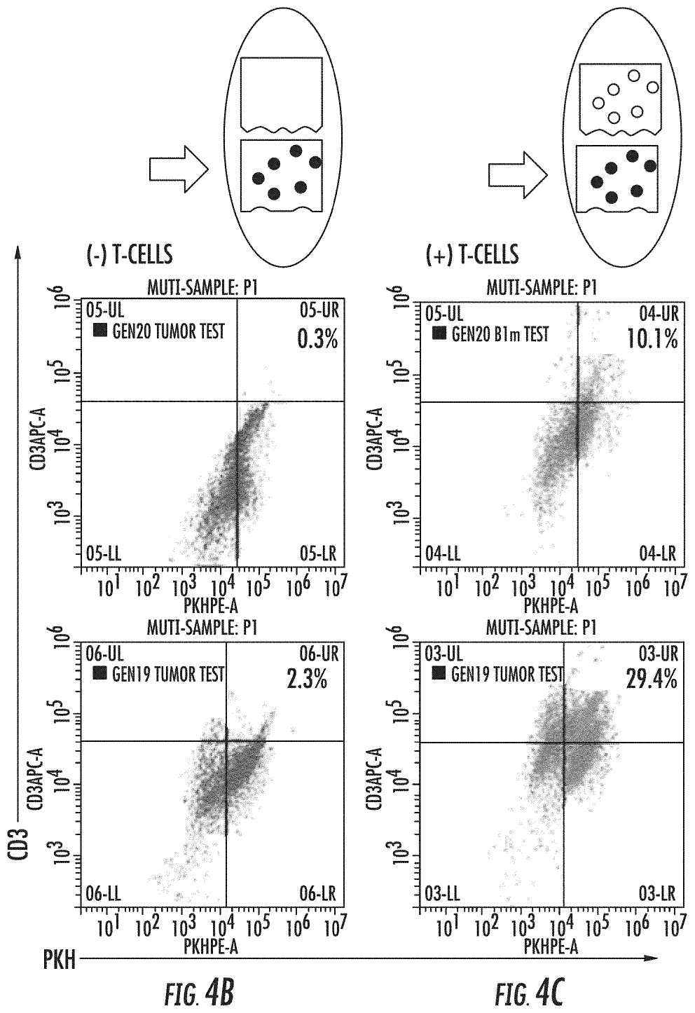

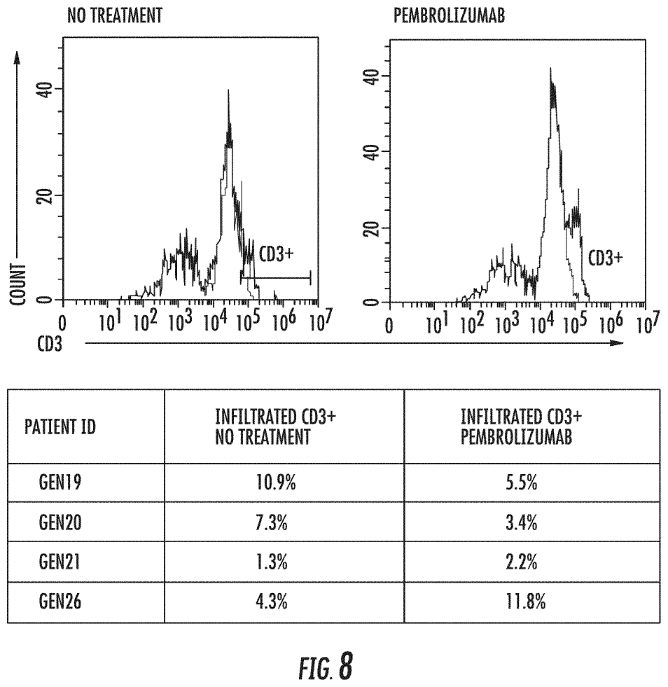

Upon detection of T cell functionality through tumor spheroid death and increased activation upon tumor spheroid stimulation, Applicant then sought to develop other methods to measure T cell function. Consequently, Applicant developed a microtumor model in order to determine if the expanded TILs are capable of infiltration. Since these expanded TILs were isolated from tumor tissue, the model should recapitulate their invasive immune phenotype. TILs were labeled with PKH stain then seeded into a compartment stacked on top of an microtumor composed of matched tumor cells (FIG. 4A). Following seven days of microtumor culture in the presence or absence of T-cells, cells were dissociated from their scaffold composed of porous ECM and evaluated using flow cytometry. TIL compartments were analyzed as a positive control for CD3+ cells that were also PKH positive for two patient samples (FIG. 4A). These gates were used to determine dual CD3/PKH positivity in microtumor samples which would be indicative of T cell infiltration. Microtumors which were cultured in the absence of a TIL compartment were first analyzed for CD3/PKH positivity as a metric to determine tumor cell specific background fluorescence (FIG. 4B). Only 0.3% and 2.3% of the events detected for the two patient samples stained positive for CD3 and PKH. Using the same gating scheme, Applicant detected a moderate level of dual CD3/PKH positivity in the GEN20 microtumor, and also detected a significantly greater amount of dual CD3/PKH staining in the GEN19 microtumor (10.1% versus 29.4%, respectively) (FIG. 4C). In addition, percent infiltration of CD3+ T cells in tumor cells of various patients in the presence and absence of treatment with pembrolizumab was also analyzed (FIG. 8). Applicants detected an increase in infiltration of CD3+ T cells with treatment for GEN21 and GEN26, while a decrease was observed with treatment for GEN19 and GEN20. These results indicate the need for screening of individual patients and the variability of biological reactions between individual patient samples. These results demonstrate that this microtumor model provides a platform to monitor TIL infiltration and decipher patient specific differences in TIL infiltration capacity.

### Example 5—Pembrolizumab Decreases Microtumor Growth Rate in a Personalized Manner

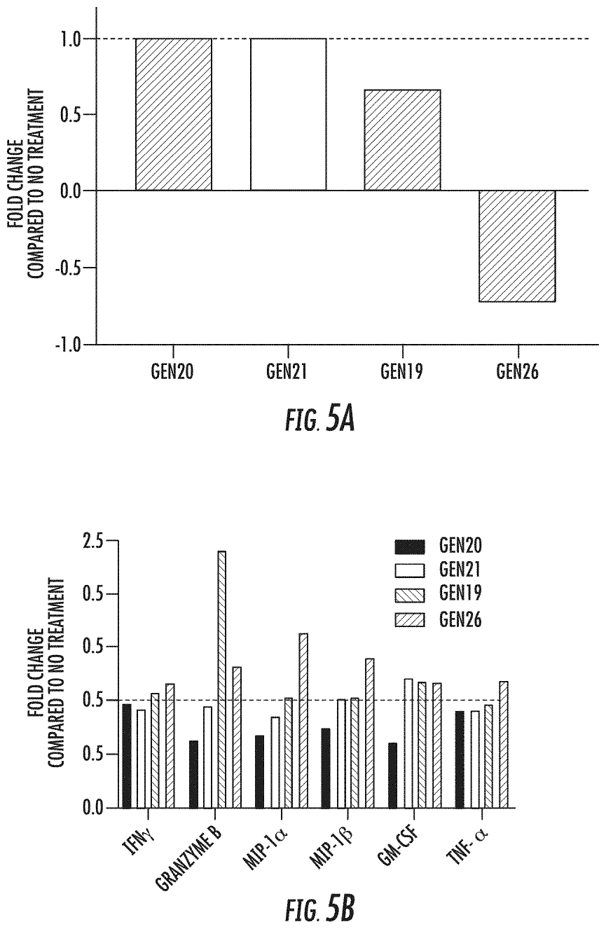

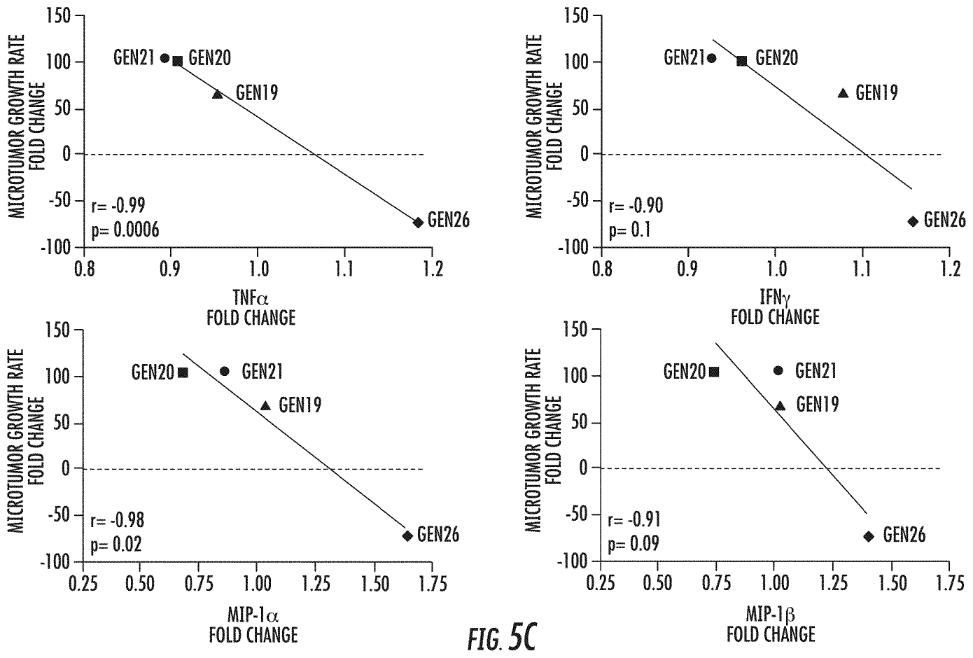

Once TIL infiltration into microtumors was confirmed, Applicant next wished to evaluate changes in microtumor viability in the presence of pembrolizumab. Applicant hypothesized the patients which displayed an increase in T cell activation in the presence of pembrolizumab in the tumor spheroid assay (FIG. 3C) would also respond in the microtumor assay via a pembrolizumab induced decrease in microtumor growth. Microtumor growth rate was determined by comparing microtumor viability at day one and day seven following co-culturing with a stacked T cell compartment that were untreated or treated with pembrolizumab. To determine fold change, pembrolizumab-treated microtumor growth rates were compared to no treatment. Applicant did not detect a difference in microtumor growth rates over the course of one week for patient samples GEN20 or GEN21 (FIG. 5A). This result was surprising for GEN21 since Applicant did detect an increase in T cell activation in the presence of pembrolizumab in the tumor spheroid assay (FIG. 3C). The lack of a difference in microtumor growth rate even in the presence of activated T cells in most likely due to differences in the infiltration of T cells into the tumor compartment (FIG. 4C). GEN19 had a decrease in microtumor viability while having a T cell infiltration of about 29.4% while GEN20 who had no decrease in microtumor viability only had a T cell infiltration of about 10%. As hypothesized for GEN19 and GEN26, Applicant did detect a pembrolizumab response via reduced microtumor growth rates compared to untreated (FIG. 5A). GEN26 resulted in a reduction in overall microtumor viability when treated with pembrolizumab compared to untreated microtumors showing that not only can pembrolizumab reduce tumor cell growth rates, but it can also reduce tumor cell viability in this system (FIG. 5A).

### Example 6—Pembrolizumab Induces Analyte Secretion which Correlates with Decreased Microtumor Growth Rate

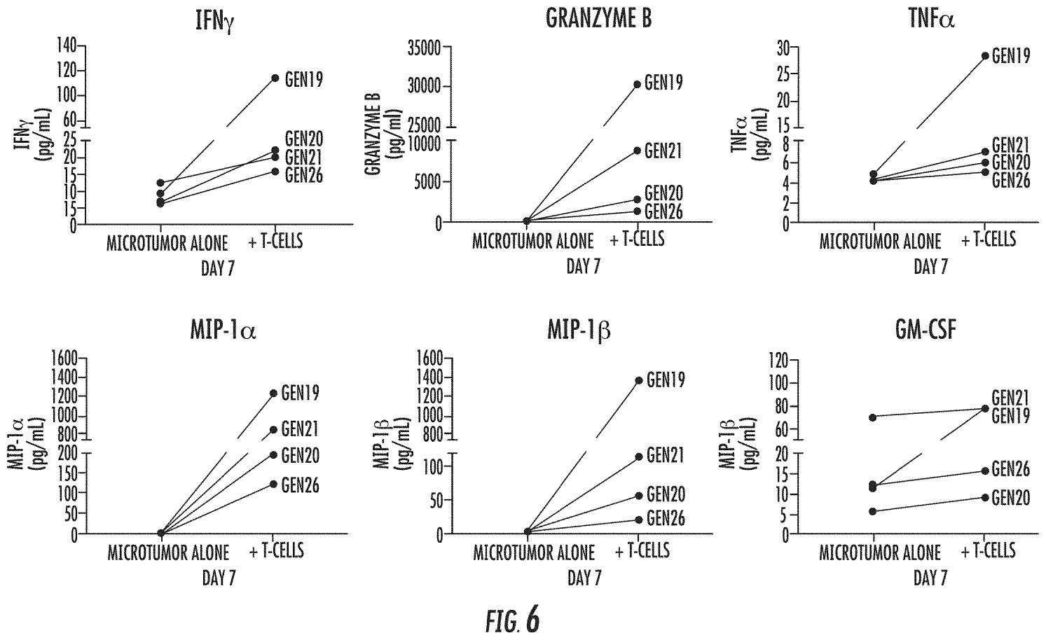

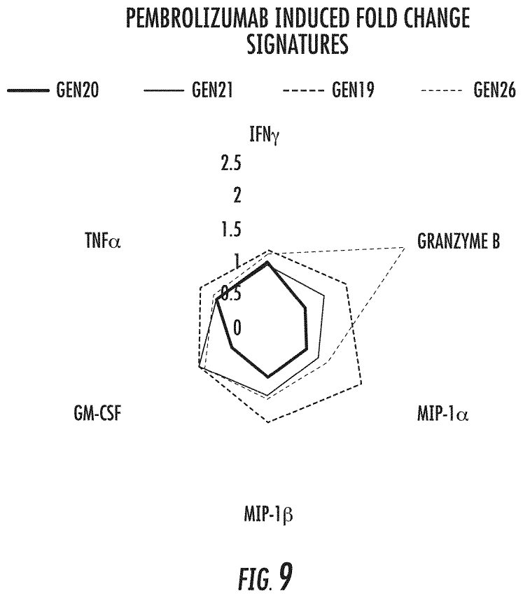

Secretions of analytes collected from supernatant at day 7 for microtumor models were then compared. All tested analytes increased when T cells were present in the system compared to microtumor alone, indicating all patient T cells were viable and functional (FIG. 6). Therefore, the lack of a pembrolizumab response for some patients was not due to non-functional T cells. Not surprisingly, the two pembrolizumab responders, GEN19 and GEN26, resulted in an increase in nearly all analytes tested following pembrolizumab treatment compared to no treatment (FIG. 5B). The greatest increase was observed from GEN19 granzyme B secretion. GEN26 displayed more consistency for increased analyte concentrations induced by pembrolizumab. GEN20 resulted in a reduction of all tested analytes following pembrolizumab treatment. Interestingly, GEN21 showed an increase in GM-CSF and moderate or no change for other tested analytes with pembrolizumab treatment which may indicate a modest response to pembrolizumab but not substantial enough to enhance a decrease in microtumor growth rates. When pembrolizumab mediated fold change in microtumor growth rates was compared with analyte secretion, Applicant found pembrolizumab-induced secretion of TNF-α and MIP-1α significantly correlated with reduction in microtumor growth rates (FIG. 5C). Additionally, IFNγ and MIP-1β showed a strong correlation, albeit not statistically significant, when the two predicative metrics were compared (FIG. 5C). Schematic showing cytokine expression signatures in different patients in response to treatment with pembrolizumab is shown based on a fold-change analysis as well (FIG. 9).

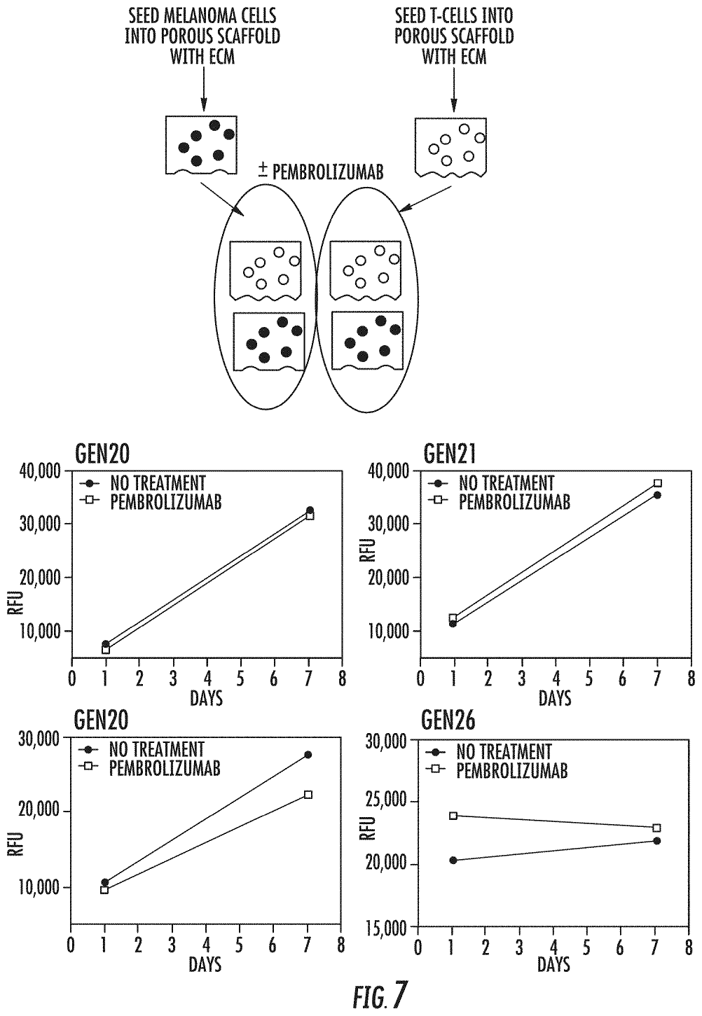

In addition, PrestoBlue absorbance readings (RFUs) for day one to day seven were measured to determine microtumor growth rates (FIG. 7). As shown in FIG. 7, pembrolizumab treatment altered the RFU readings over the 7 day period for GEN19 and GEN26 more significantly than GEN20 and GEN21.

These results demonstrate the microtumor model can detect pembrolizumab responses in a personalized manner via effects on microtumor growth and cytokine secretion.

### Example 7—Methods

Melanoma tumor cell and T cell isolation and expansion from human primary tissues. Seven frozen primary human melanoma tissues were purchased from GHS Biorepository. Generation of single-cell suspensions from tissue chunks was executed using the enzymatic digestion. Following single cell isolation, cells were separated for tumor cell expansion in EV3D media or for T cell expansion in ImmunoCult™-XF T cell Expansion Media (StemCell™ Technologies 10981) supplemented with Immunocult™ Human CD3/CD28 T cell Activator (StemCell™ Technologies 10971) according to manufacturer's recommendations and 50 ng/ml interleukin-2 (Roche 11011456001). T cells were expanded for approximately two weeks, and then frozen until needed for downstream experiments. Tumor cells were either expanded to sufficient numbers for experimentation or frozen after one passage to be utilized at a later date. PD-L1 expression on tumor cells or CD3/CD8 population detection in T-cell expansions was determined following expansion and prior to being frozen or assayed as described under flow cytometry. Antibodies: PD-L1-PE (BO 557924, 1:20), PE Mouse IgG1, kappa Isotype control (BO 555749, 1:20), CD3-APC (Miltenyi 130-113-135, 1:100), REA control APC (Miltenyi 130-104-614, 1:20), CD8-PerCP-Vio700 (Miltenyi 130-110-682, 1:20), REA control (S)-PerCP-Vio700 (Miltenyi 130-113-441, 1:20).

Tumor spheroid death assay. Expanded T cells at different densities (2.5×10³, 6.25×10³, 12.5×10³, 25×10³, or 50×10³) were seeded in the presence or absence of patient matched tumor cells (2.5×10³) with or without the addition of 100 μg/ml pembrolizumab (SelleckChem A2005) as six or seven technical replicates in a 384-well round bottom ultra-low attachment plate (Corning 3830). The cell death control, 10% DMSO, was added at the same time as pembrolizumab. After a 1 hour incubation at 37 C 5% CO₂, plates were centrifuged for 5 minutes at 500×g. Tumor spheroids were allowed to form overnight. After 24 or 72 hours, CellTiter-Glo® 3D Cell Viability Assay (Promega G9683) was used according to manufacturer's recommendations and relative luminescence units (RLUs) were recorded using a TEGAN infinite M1000pro (Mannedorf, Switzerland). RLUs were graphed using GraphPad Prism.

Tumor spheroid assay to assess T cell responses. Expanded T cells were cultured in the absence of activation components (EV3D media) for 72 hours prior to tumor spheroid assay. T cells were seeded in the presence or absence of patient matched tumor cells with or without the addition of indicated concentration of pembrolizumab (SelleckChem A2005) as seven technical replicates in a 384-well round bottom ultra-low attachment plate (Corning 3830). After a 1 hour incubation, plates were centrifuged for 5 minutes at 500×g. Patient samples were then cultured for 24 hours, and wells were then harvested for flow cytometric analysis.

Flow cytometry. Cells were harvested from 384-well plates following tumor spheroid assays or dissociated from scaffolds and resuspended in 100 μl FACS buffer (2% FBS, 2 mM EDTA, 0.05% sodium azide in PBS). Test antibodies PD-1-PE-Vio770 (Miltenyi 130-117-698, 1:100), CD3-APC (Miltenyi 130-113-135, 1:100), CD8-PerCP-Vio700 (Miltenyi 130-110-682), CD4-FITC (Miltenyi 130-114-531, 1:100), CD25-PE (Miltenyi 130-115-534, 1:100), CD69-APC-Vio770 (Miltenyi 130-112-616, 1:100) or isotype controls IgG2b-PE-Vio770 (Miltenyi 130-096-825, 1:100), REA control (S)-APC (Miltenyi 130-104-614, 1:20), REA control (S)-PerCP-Vio700 (Miltenyi 130-113-441, 1:100), REA Control (S)-FITC (Miltenyi 130-113-437, 1:20), REA Control (S)-PE (Miltenyi 130-104-612, 1:20), REA Control (S)-APC-Vio770™ (Miltenyi 130-104-618, 1:20) were added and incubated for 10 minutes at 4° C. Samples were diluted to 200 μl of FACS buffer and centrifuged 500×g for 5 minutes. Cells were washed in 100 μl of FACS buffer, centrifuged again, then resuspended in 100 μl of FACS buffer. Samples were then analyzed using the CytoFLEX LX flow cytometer and software (Beckman Coulter, Brea, Calif.). Percent of parent was then graphed and evaluated for statistics using GraphPad Prism.

Detection of T cell infiltration. T cells were stained with the fluorescent stain PKH (Sigma PKH26GL-1KT) according to manufacturer's recommendations. On the same day, T cells and tumor cells were seeded in EV3D media and Matrigel (Corning 356234) in scaffolds. Scaffolds were incubated for 1 hour at 37° C. and 5% CO₂ then 200 μl of EV3D media was added to each scaffold well. After an additional hour, T cell scaffolds were placed on top of tumor cell scaffolds (microtumors). Media was changed every one or two days. Viability was determined for T cell scaffolds and microtumors after one week using PrestoBlue™ Cell Viability Reagent (Invitrogen A13262). Microtumors were washed in PBS, then harvested using Liberase DH (Sigma LIBDH− RO) in serum-free media while rotating at 37° C. 5% CO₂. Harvested cells were then analyzed for PKH (PE channel), and CD3 using flow cytometry. Unstained T cells were used to define PKH+ gates.

PrestoBlue viability assays. PrestoBlue™ Cell Viability Reagent (Invitrogen A13262) was diluted 1:10 in EV3D media. Each T cell scaffold or microtumor was placed in 250 μl of diluted PrestoBlue reagent in a 96-well plate and allowed to incubate for 2 hours at 37° C. 5% CO₂. Scaffolds and microtumors were then placed in fresh media and duplicates of 100 μl per PrestoBlue sample was pipetted into a black walled 96-well plate and read on a TECAN infinite M1000pro (Mannedorf, Switzerland) at an excitation of 560 nm and an emission 590 nm with a gain of 60. Absorbance measurements from scaffolds with no cells were subtracted from each sample and relative fluorescent units (RFU) were graphed using GraphPad Prism.

Microtumor viability assay. T cells and tumor cells were seeded in EV3D media and Matrigel (Corning 356234) with collagen (Corning 354236) in scaffolds. For checkpoint inhibitor treated groups, pembrolizumab (SelleckChem A2005) was seeded within the T cell scaffolds and microtumors at a final concentration of 100 μg/ml. Scaffolds were incubated for 1 hour at 37° C. and 5% CO₂ then 200 μl of EV3D media was added to each scaffold. After an additional hour, T cell scaffolds were placed on top of tumor cell scaffolds (microtumors) or microtumors were cultured in the absence of a stacked T cell scaffold. Media was changed every one to two days. Supernatant from day seven was collected and stored at −80° C. for analyte analysis. Viability was determined for T cell scaffolds and microtumors after 24 hours (day one) and at day seven using PrestoBlue™ Cell Viability Reagent (Invitrogen A13262). PrestoBlue absorbance readings (RFUs) for day one were subtracted from day seven to determine microtumor growth rates. Fold change in pembrolizumab treated microtumor growth rates were determined by dividing treatment group growth rates by no treatment group growth rates.

Detection of Analyte Secretion. Individual beads (GM-CSF, IFNγ, Granzyme B, MIP-1α, MIP-1β, TNFα) were purchased from the Milliplex™ MAP Human CD8+ T cell Magnetic Bead Panel 96-well Plate Assay (EMO Millipore HCD8MAG-15K). Supernatant was collected at day seven of microtumor viability assays and stored at −80° C. Samples were processed according to manufacturer's recommendations and the assay plate was run on the BIORAD Luminex® BioPLEX200™ System (Hercules, Calif.). Analyte concentration was determined by interpolation of assay standards using GraphPad Prism. Fold change analyte secretion was determined for pembrolizumab treated samples by dividing them by the analyte concentrations for no treatment samples.

Statistical Analysis. Unless stated otherwise, results are expressed as the means±standard deviation (SD). Unpaired t tests were used to determine significance between two groups. Unpaired One-way ANOVA was used to determine significance across three or more groups. Pearson correlations were determined using GraphPad Prism.

### Example 8—Predicting Patient Response to Checkpoint Blockade Therapy Using In Vitro 3D Culture

Knowledge of immune responses that correlate with clinical outcome is essential for the development of strategies to harness a patient's immune system to eradicate cancer. Pre-clinical platforms that recapitulate the immune response in the context of cancer are necessary for adequate understanding and detection of clinical efficacy, however, the technology to accurately test immuno-oncology (I/O) therapy response is lacking. Despite the value animal models provide in a pre-clinical setting, they lack matched patient tumor and immune cell interactions. To address this shortcoming, Applicant developed in vitro 3D tissue models that maintain autologous patient tumor cells and immune cells for the testing and prediction of immune cell responses. Applicant hypothesize that these 3D tissue models will recapitulate the patient tumor microenvironment and detect response to I/O agents.

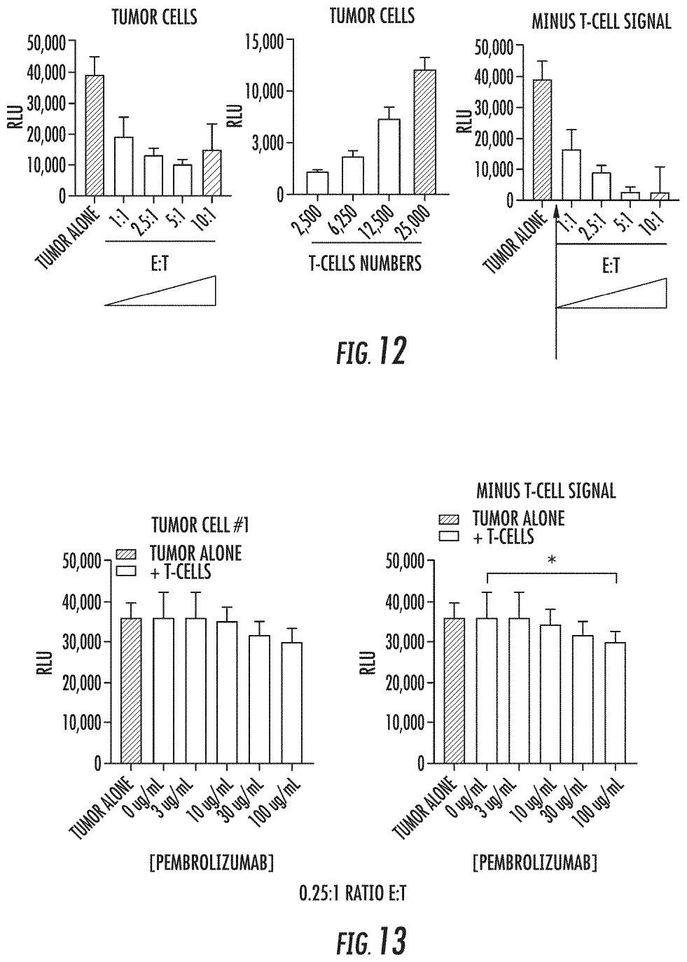

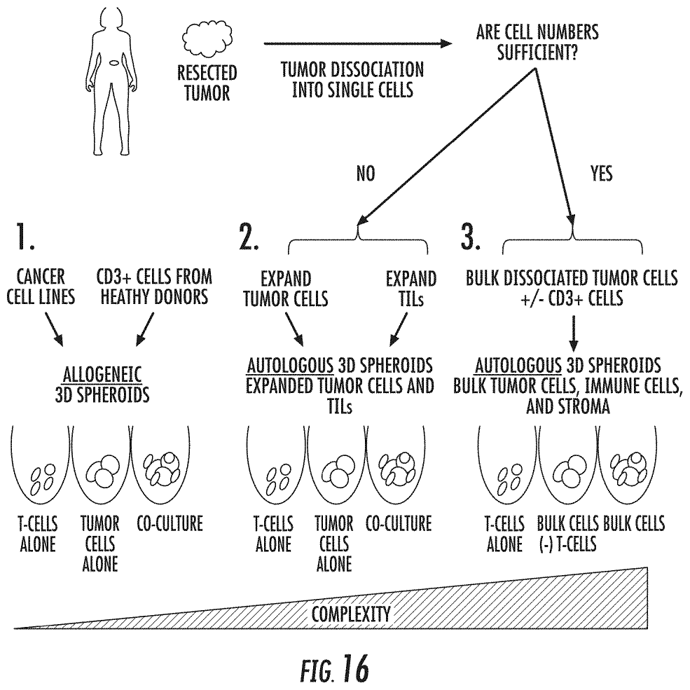

Referring to FIG. 16, tumor cells and T cells were obtained from seven melanoma patient biopsies and screened for PD-L1 and lymphocyte populations prior to incorporation into 3D culture. Effector cell to Tumor cell (E:T) optimization assays were conducted with expanded T cells at different densities and co-cultured at different time points with tumor cells. See FIGS. 12-13 for data showing the results of screening for the most effective effector cell (E) to tumor cell (T) ratios and the results of therapy response using tumor infiltrating T lymphocytes, where data is shown at 24 hours.

Viability was measured using CellTiter-Glo® 3D. T cell response was determined using flow cytometry following 24-hour co-culture with tumor cells.

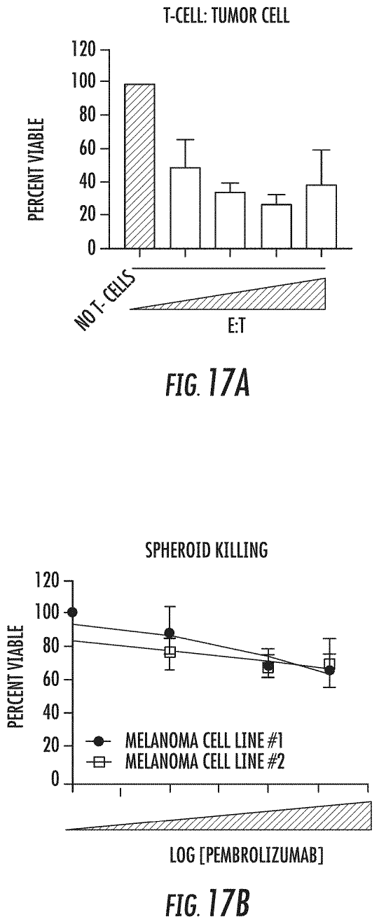

FIGS. 17A-17C show a dose-dependent response to checkpoint blockade in 3D cell line spheroids. FIG. 17A shows effector cell (T cell) to target cell (tumor cell) (E:T) ratio screens with T-cells from healthy donors. From these data, a single ratio was selected for drug response profiling. FIG. 17B shows T cell mediated killing of tumor spheroids for two melanoma cell lines after 24 hour treatment with Pembrolizumab. Results of Atezolizumab and Durvalumab tumor spheroid killing in the presence or absence of T cells using a NSCLC cell line for 24 hours are shown in FIG. 17C.

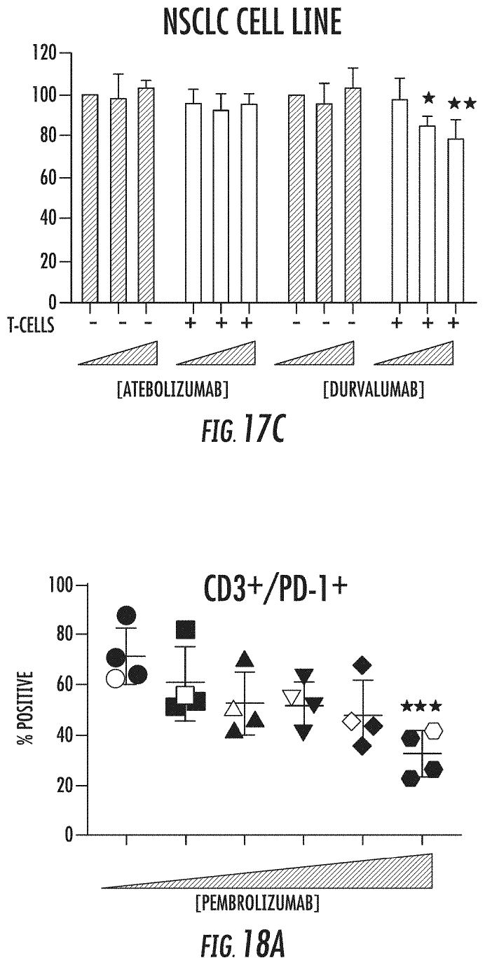

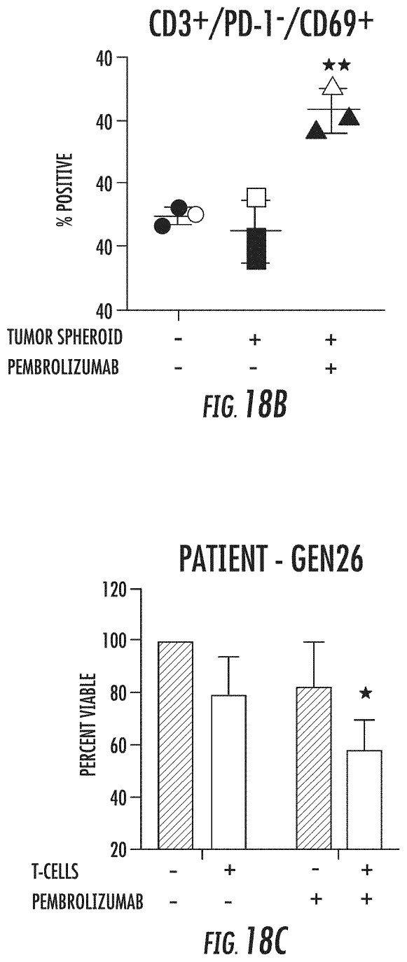

FIGS. 18A-18C show that pembrolizumab binds and activates expanded TILs ex vivo, inducing patient-specific tumor cell death. Following 72 hours of treatment with Pembrolizumab drug occupancy determined for T cells (FIG. 18A). FIG. 18B shows data for CD3+/PD-1−/CD69+ T cells compared to T cells stimulated with spheroids ±100 μg/mL of pembrolizumab. GEN26 spheroids were seeded ± matched T cells and treated ±100 μg/mL pembrolizumab for 24 hrs (FIG. 18C).

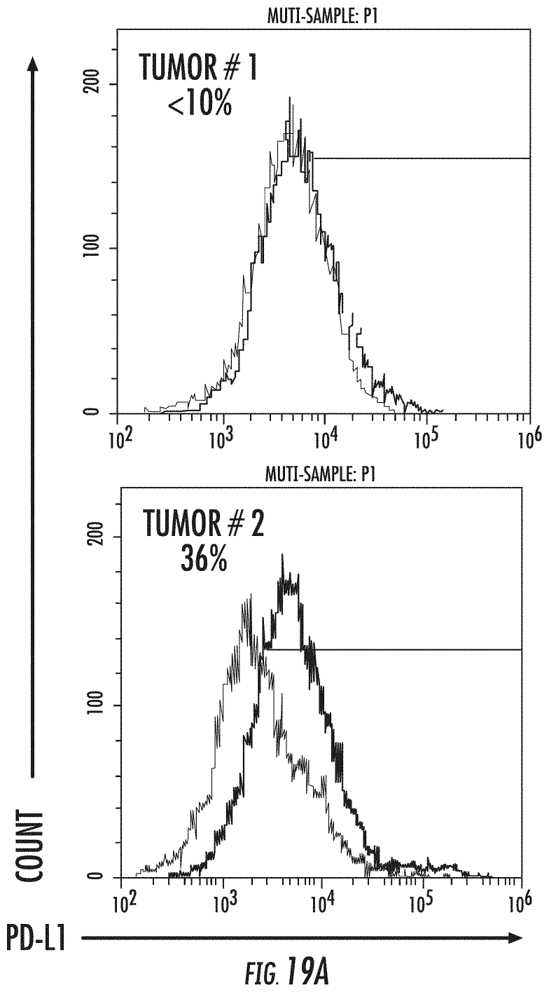

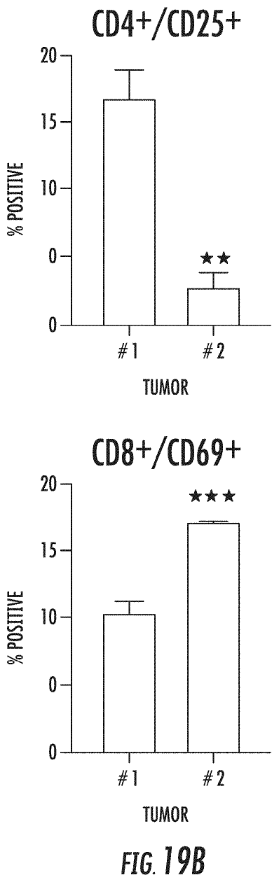

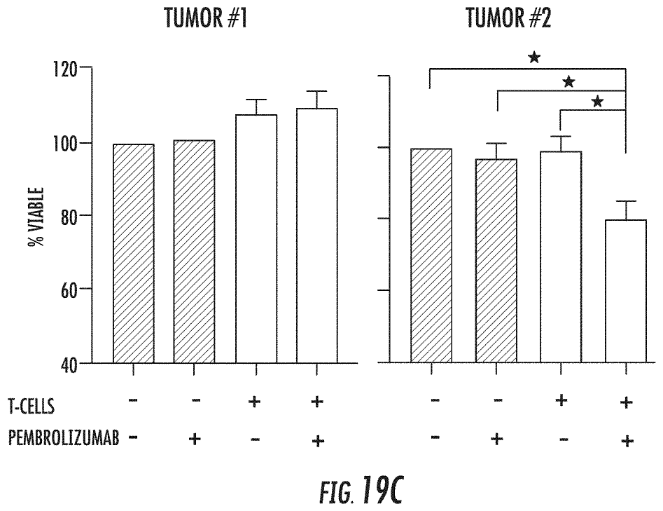

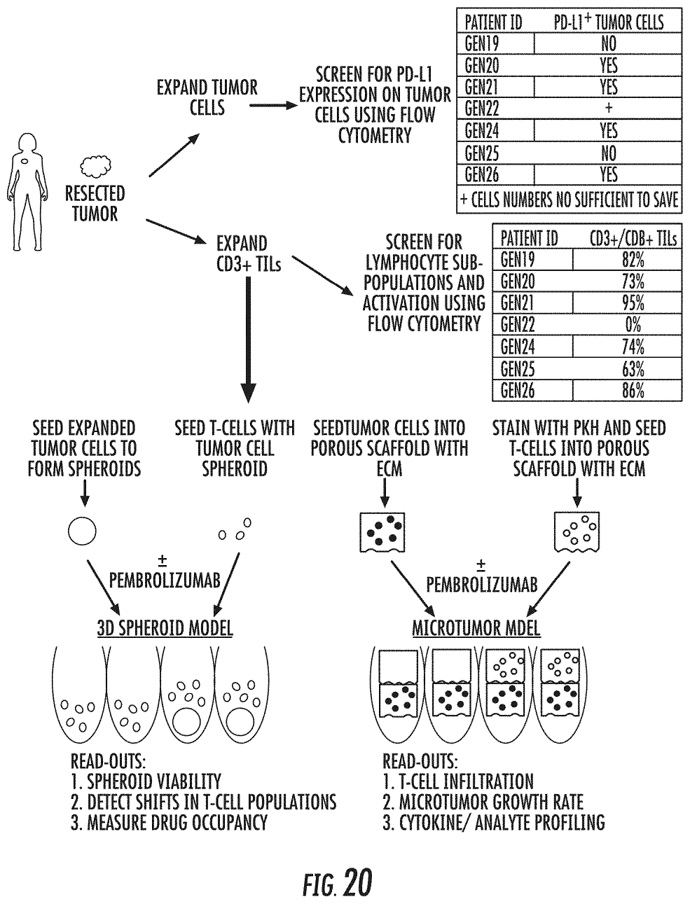

Tumor samples were dissociated into single cells and tested for PD-L1 expression using flow cytometry (FIG. 19A). T cell populations from within the dissociated tumor cells were analyzed for CD4+/CD25+ positivity and for CD8+/CD69+ positivity. Results are shown in FIG. 19B. Spheroids were treated with or without 300 μg/mL of pembrolizumab and spheroid viability was determined after 24 hrs (FIG. 19C). The results suggest that the high throughput autologous tumor spheroid model is capable of detecting T cell mediated spheroid death, pembrolizumab occupancy, and shifts in T cell population induced by tumor spheroid stimulation and pembrolizumab treatment. These in vitro 3D platforms are suitable and complimentary for preclinical testing of new I/O agents.

### Example 9—Predicting Patient Response to Immune-Oncology Agents In Vitro Using 3D Cultures

Immuno-oncology (I/O) based therapeutics, including cellular therapies and checkpoint inhibitors have surged in the last 2 years, but the technology to accurately test them in a pre-clinical setting is significantly lacking. While animal models have tried to provide accurate testing platforms, the ultimate goal of a matched patient tumor and immune system is not achievable in mice. To overcome this issue, Applicant has developed two 3D tissue systems for in vitro testing that combine a patient's tumor cells and autologous immune cells for accurate testing and prediction. Applicant hypothesizes that the 3D cell culture systems can recapitulate the patient's tumor microenvironment to detect I/O response. Applicant's spheroid-based system allows for monitoring of how primary T cells are affected by paired tumor cells and/or the PD-1 inhibitor pembrolizumab using flow cytometry. Applicant has successfully detected pembrolizumab binding to T cells in a dose dependent manner, clonal expansion of lymphocyte populations, as well as increased expression of activation markers on CD3+ cells following combination with tumor cells and exposure to pembrolizumab. This model also accurately detects CD3+CD8+ T cell mediated tumor cell death and can be used to track changes in secreted cytokines and chemokines such as Granzyme B and IFNγ. Applicant's second model, a 3D microtumor platform, allows to detect immune cell migration and infiltration and therapy related cell death. The results show pembrolizumab can increase lymphocyte infiltration while simultaneously decreasing microtumor growth in matched patient samples whose tumor cells express PD-L1 and whose lymphocytes are CD8+. Cytokine secretion detected by multiplex technology from the microtumor model supports the observed enhanced T cell activation in the presence of pembrolizumab. The data generated from the two complex 3D in vitro models can recapitulate in vivo biology in order to derive correlations to I/O drug response. These models can be utilized for preclinical testing of new I/O agents as well as for patient response predictions to I/O therapies.

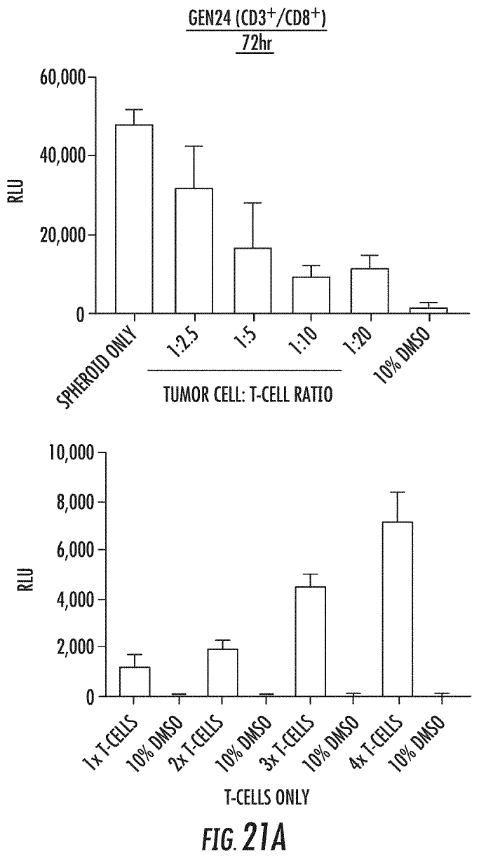

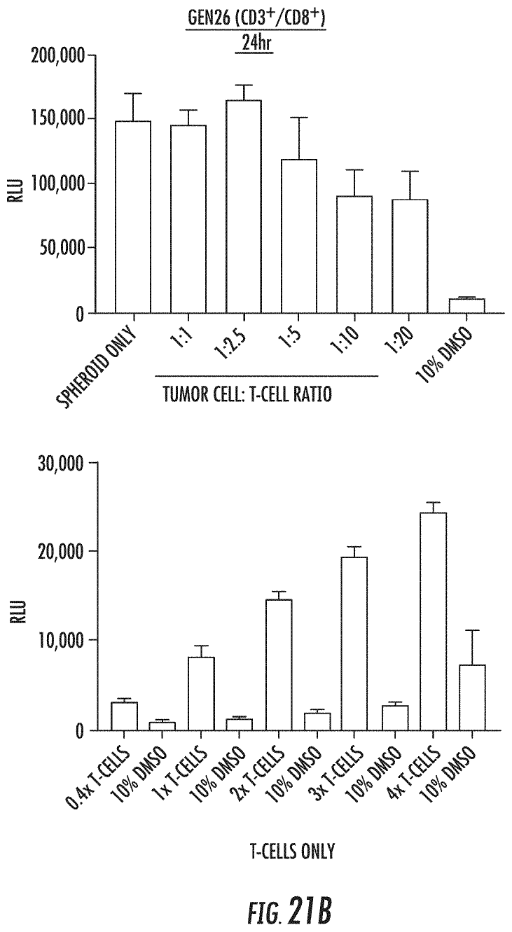

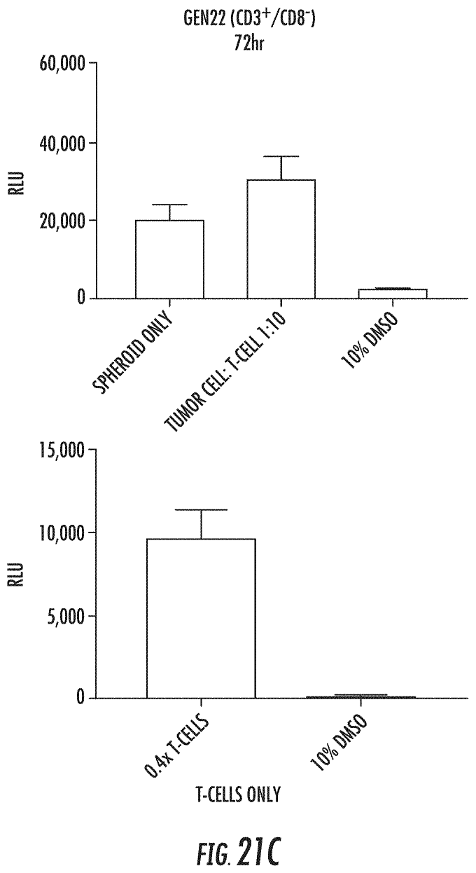

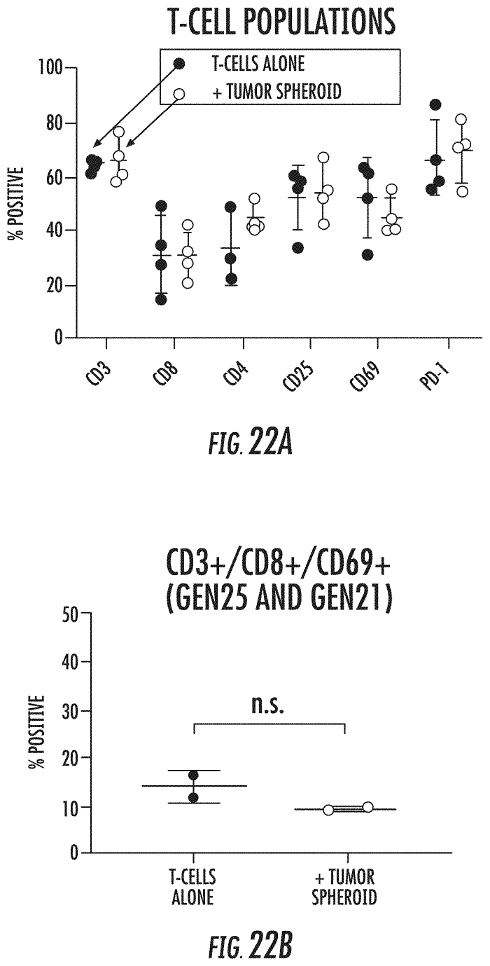

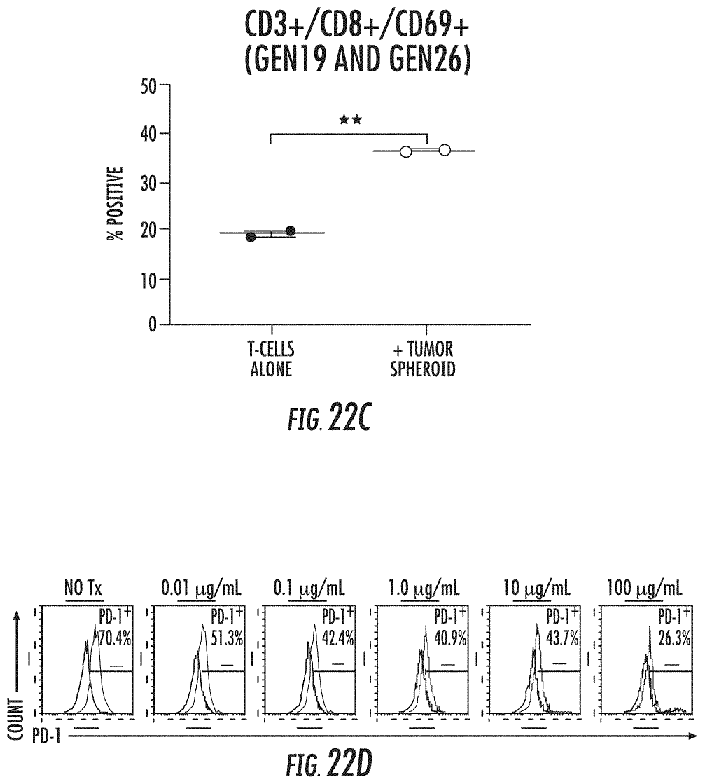

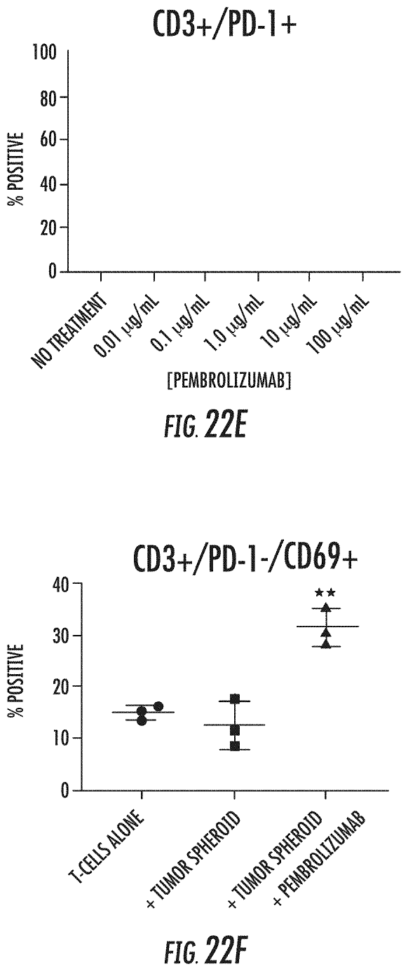

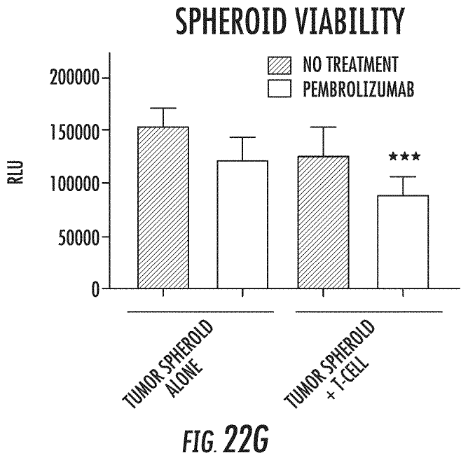

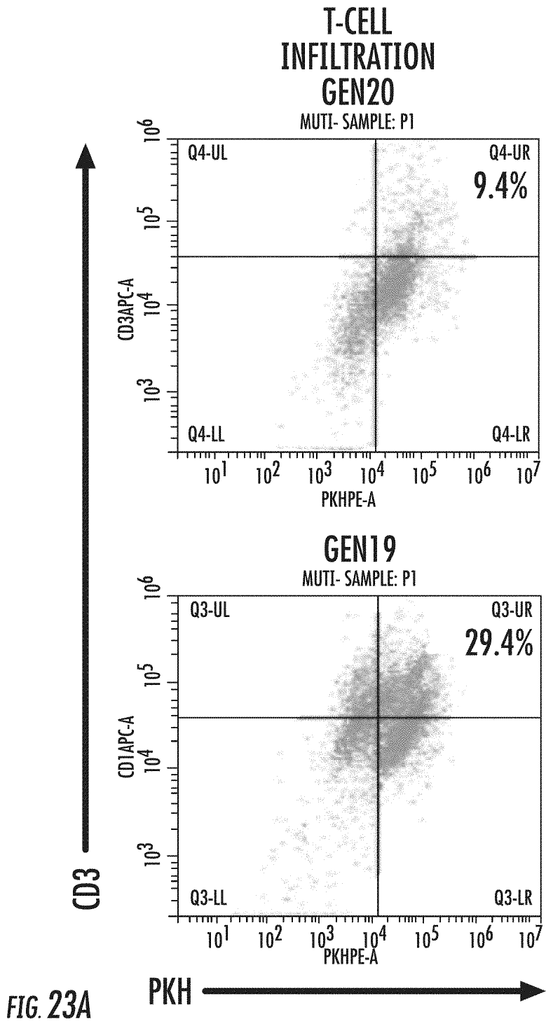

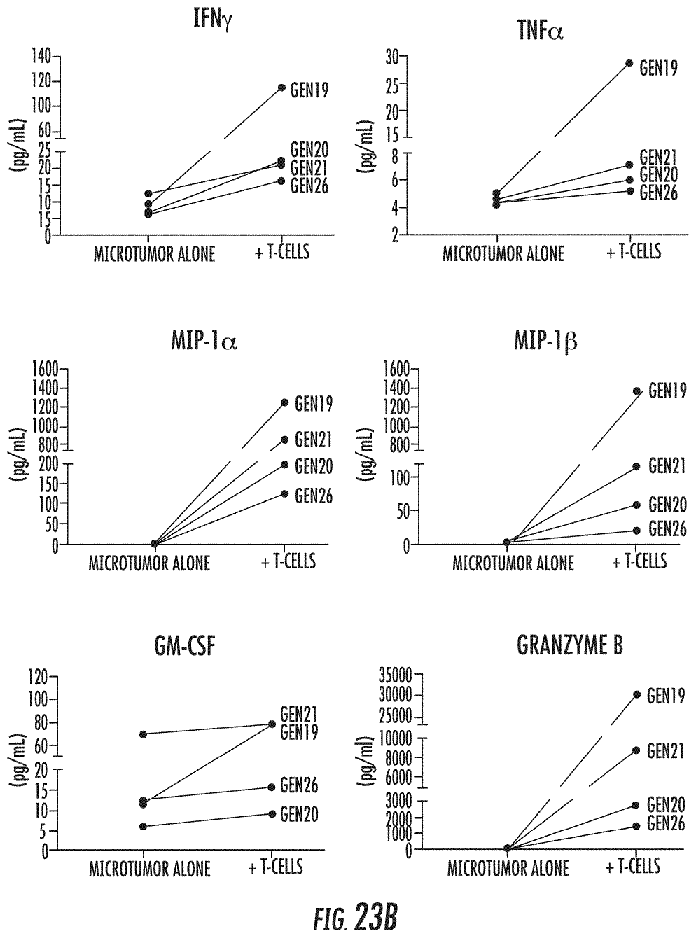

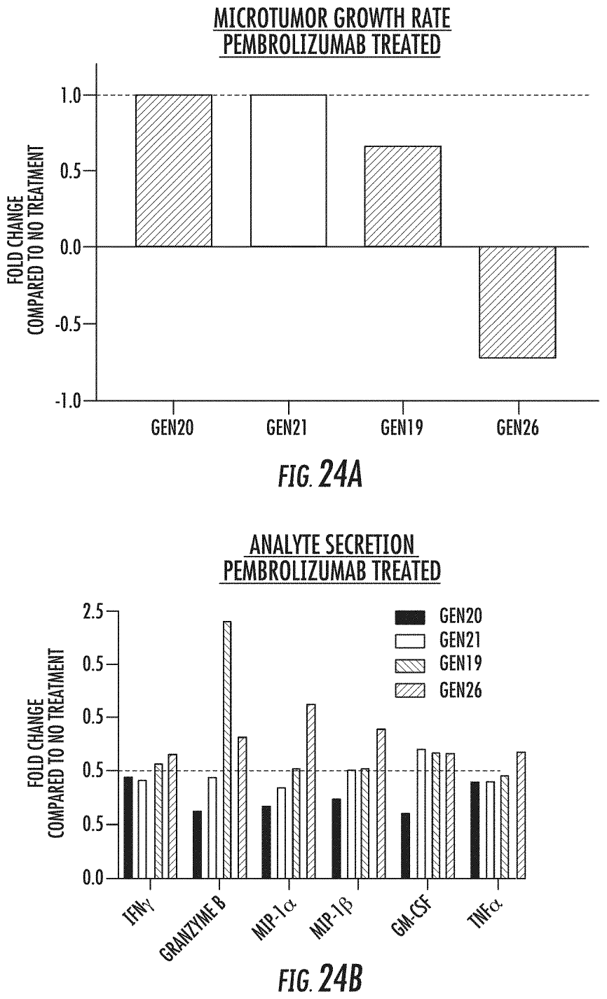

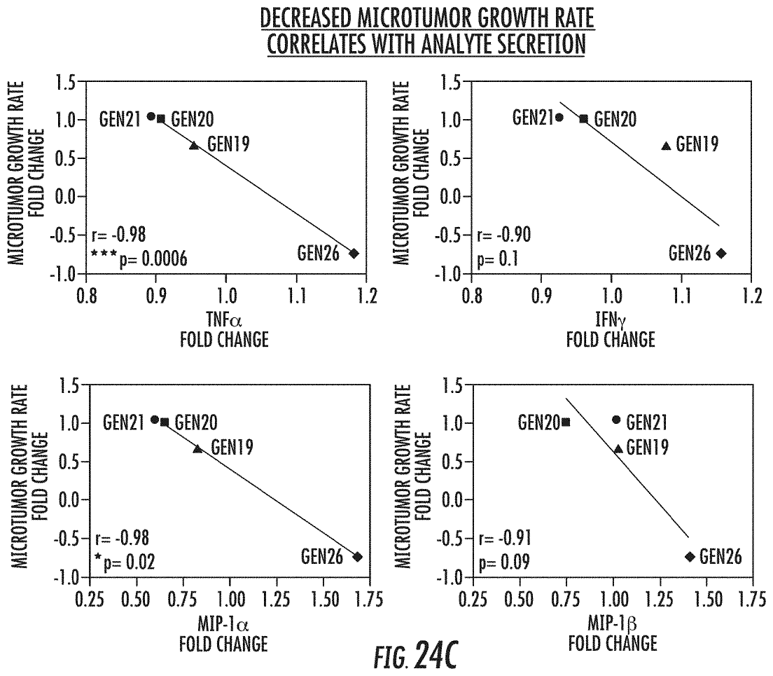

A schematic for the autologous 3D model is illustrated in FIG. 20. FIGS. 21A-21C illustrate how the model can be used to induce cytotoxic T cell-mediated tumor spheroid death. T cell population shifts as a result of treatment of spheroid cells with pembrolizumab were detected as shown in FIGS. 22A-22G. Treatment with pembrolizumab also induced analyte secretion and T cell infiltration in the microtumor model (FIGS. 23A, 23B). As such, microtumors aid in predicting response of tumor cells to pembrolizumab (FIGS. 24A-24C).

The high throughput autologous tumor spheroid model is capable of detecting T cell mediated spheroid death, pembrolizumab occupancy, and shifts in T-cells population induced by tumor spheroid stimulation and pembrolizumab treatment. The microtumor model is also capable of detecting patient-specific T cell infiltration, and therapy mediated reduction of microtumor growth rate. Microtumor analyte secretion was shown to correlate with treatment response. These two in vitro 3D platforms are ideal and complementary for preclinical testing of new I/O agents as well as patient response predictions to I/O therapies.

### Example 10—Multifaceted Functional Assessment of Checkpoint Inhibitor Efficacy Using 3D Tumor Spheroids

As described above three-dimensional (3D) culture of immortalized cell lines and patient-derived primary cells has been shown to be a more representative in vitro model of tumor biology compared to standard 2D techniques. For immune-oncology, animal models continue to lack the ability to fully recapitulate the human immune system. The use of 3D models to study immunotherapies provides the opportunity to mimic the complex interactions between immune cells and the tumor microenvironment in a fully human system. However, standard well-based assays that measure cell viability prevent the obtainment of useful knowledge on tumor-immune cell interactions and immunotherapy effect on immune cell numbers and viability. To address these limitations, Applicant developed an in vitro based assay for the visualization and quantitation of T-cell-mediated cell death using fluorescently labeled live tumor cells and T-cells in a 3D spheroid platform. Enhanced T-cell mediated tumor cell killing using CD3/CD28 activator and anti-PDL1 drug, Durvalumab, can be measured allowing for real-time evaluation of tumor-specific apoptosis in the presence of cytotoxic T-cells.

In order to better understand tumor/T-cell interactions in the 3D model, Applicant cultured primary tumor cells in 3D and measured changes in T-cell populations, such as T-cell activation, and changes in CD8+:Treg ratios, induced by tumor spheroids with or without T-cell conditioned media treatment using flow cytometry. In order to detect changes to tumor biology induced by T-cells as well as immune-oncology drug treatments, Applicant monitored dynamic fluctuation of PD-L1 using immunofluorescence in the spheroids. Additionally, using TUNEL staining, Applicant confirmed that tumor cell death was T-cell mediated. In summary, Applicant was able to observe T-cell mediated tumor cell killing and detect changes in biomarker expression and immune cell composition in response to changes in culture conditions. This multifaceted approach is ideal for the functional evaluation of preclinical I/O agents and for identifying drug combinations and timepoints, as it represents a holistic perspective of drug response.

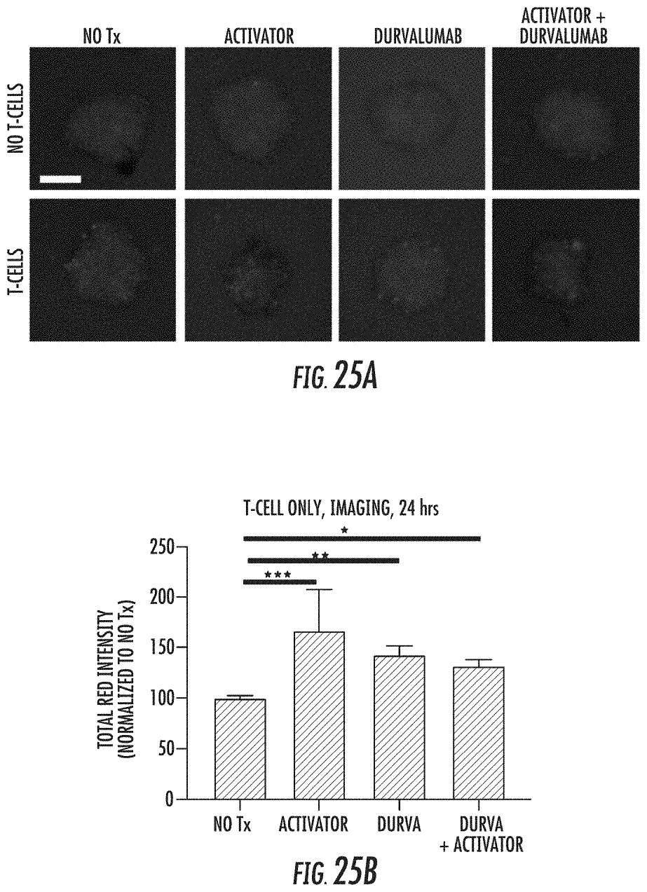

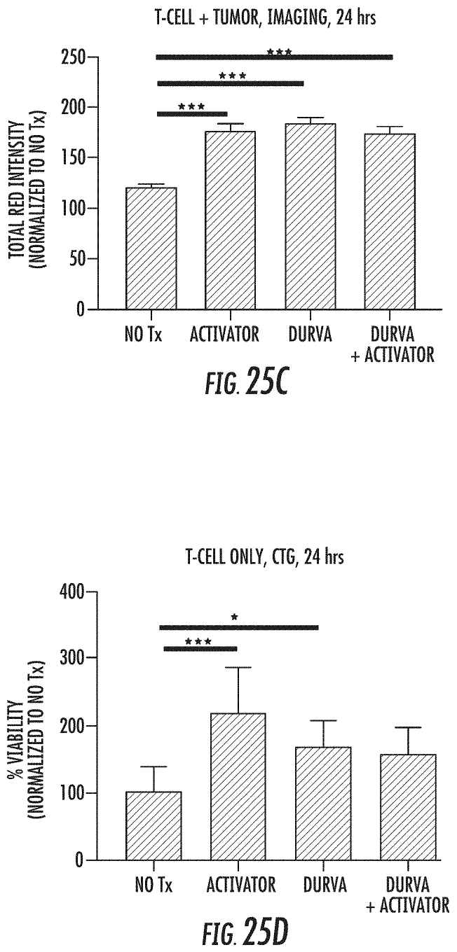

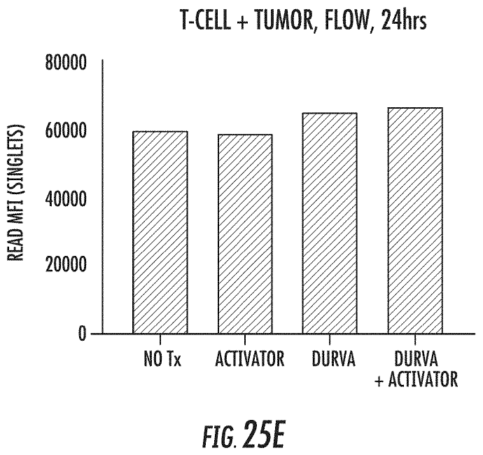

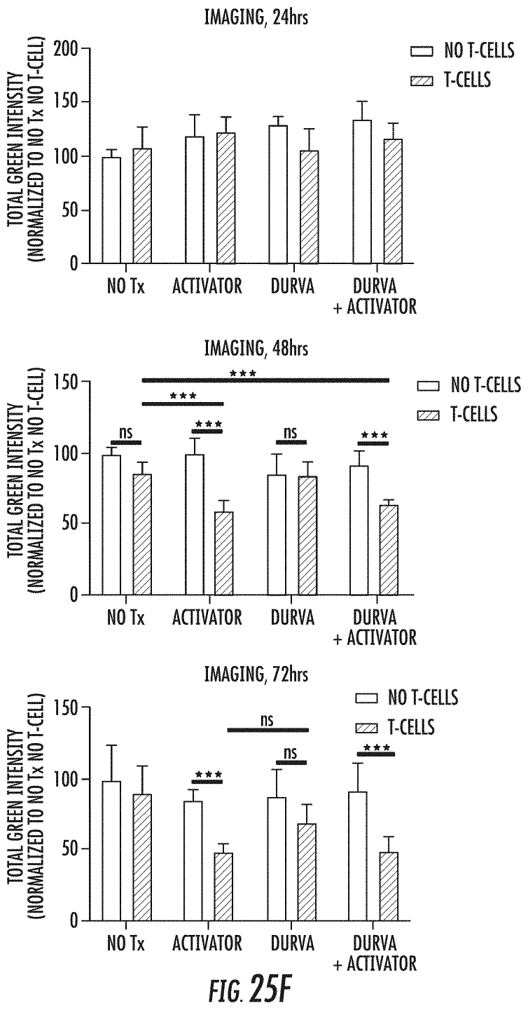

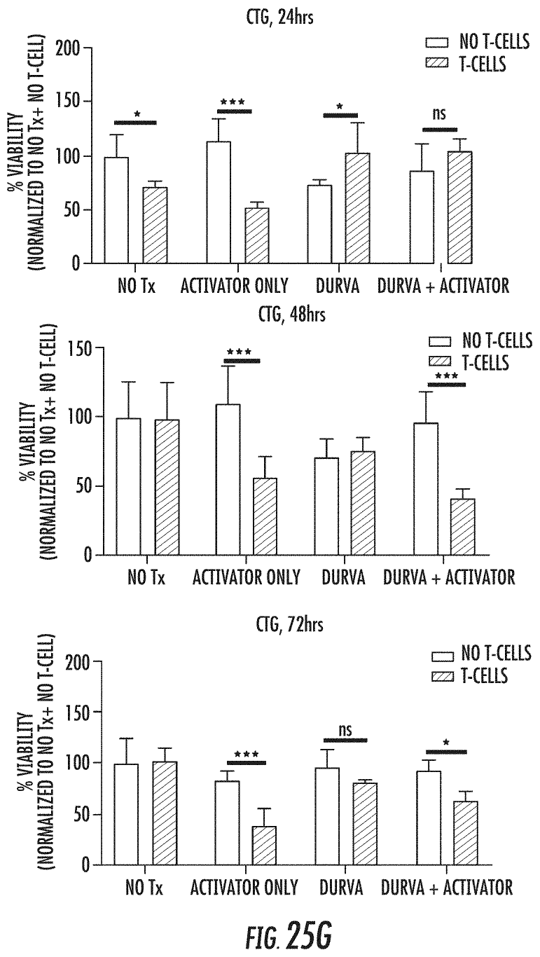

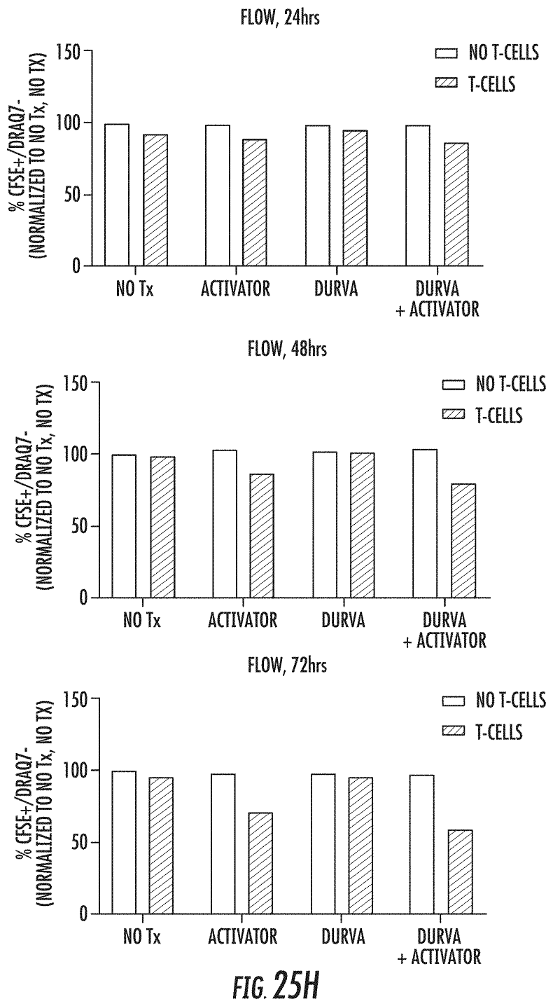

Referring to FIGS. 25A-H, a summary of an image-based assay for measuring tumor and T-cell interactions for I/O applications is shown. FIG. 25(A) illustrates representative images of a melanoma cell line (Green) and tumor infiltrating lymphocytes (Red) at a 1:3 Effector:Target cell ratio (E:T) cultured as 3D spheroids at 72 hours, Scale bar=200 μm. Groups were treated with immunomodulators (Activator=CD3/CD28 antibody, Durva=Durvalumab) and analyzed every 24 hours for 72 hours (FIGS. 25F-25H). Total red or green fluorescence was measured for T-cells (FIGS. 25B and 25C) and tumor cells (FIG. 25F) using an algorithm developed in Celleste Image Analysis software. Imaging based metrics were compared to conventional methods for assessing cell viability, CellTiter-Glo® (FIGS. 25D and 25G) and flow cytometry (FIGS. 25E and 25H). An increase in T-cell viability in all treatment groups was observed compared to no treatment when cultured with tumor cells (D). Total green fluorescence signal (F) was an acceptable indicator of T-cell induced cytotoxicity when compared to CellTiter-Glo® (FIG. 25G). The use of flow cytometry as a metric for T-cell induced cytotoxicity appears to not be as sensitive at earlier timepoints (24 hrs) as at later timepoints (72 hours). The assay was able to detect changes in tumor viability and T-cell proliferation due to a known T-cell activator (CD3/CD28). Durvalumab alone did not cause significant T-cell induced cytotoxicity. Two-Way ANOVA, error bars reflect SD. *p<0.05, **p<0.01, ***p<0.001.

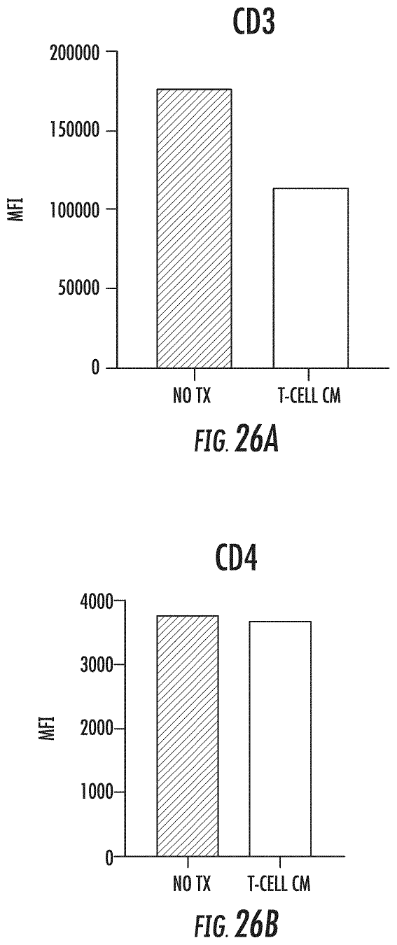

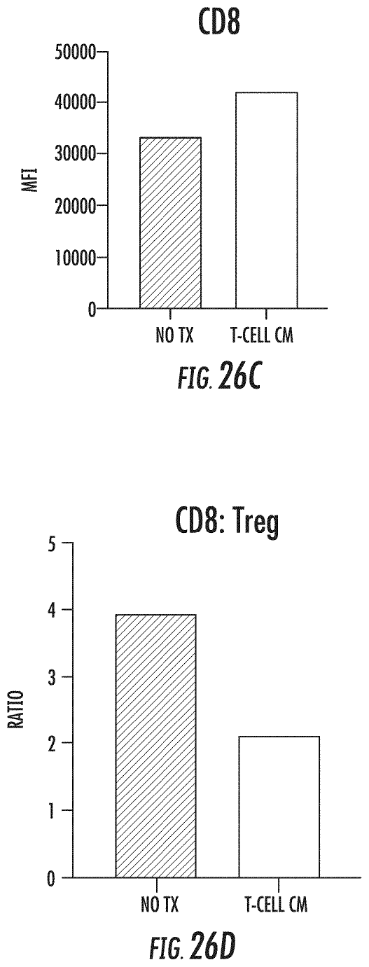

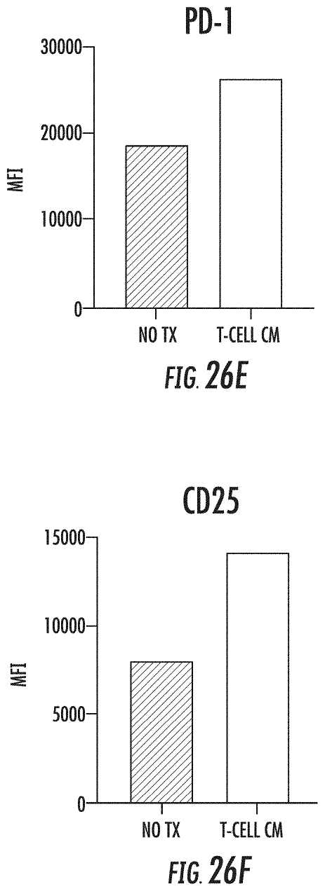

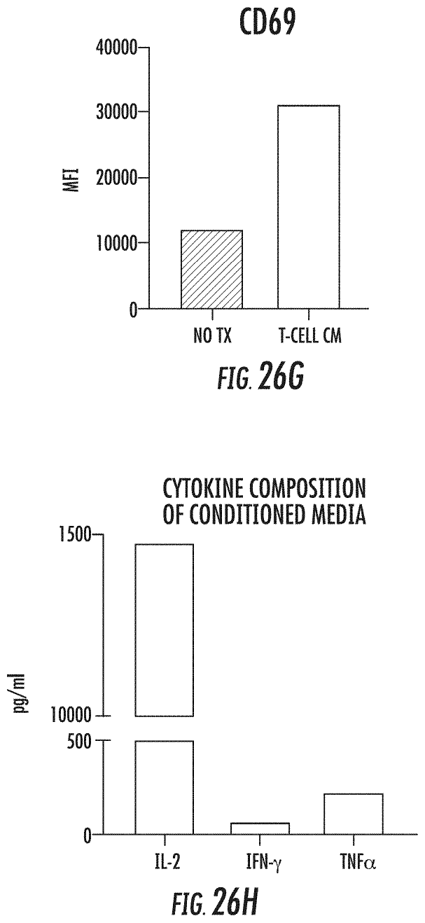

FIGS. 26A-H illustrate T-cell conditioned medica induced changes of immune cell composition of primary tumor tissue. Dissociated cells from a primary ovarian tumor were cultured in 3D with or without T-Cell Conditioned Media (T-cell CM) for 72 hours. Dissociated spheroids were analyzed by flow cytometry. The results showed a decrease in CD8:Treg (FIGS. 26A-26D) and an increase in T-cell activation (FIGS. 26E-2G) in response to T-Cell CM. Cytokines present in CM included IL-2, IFN-γ, and TNFα (H).

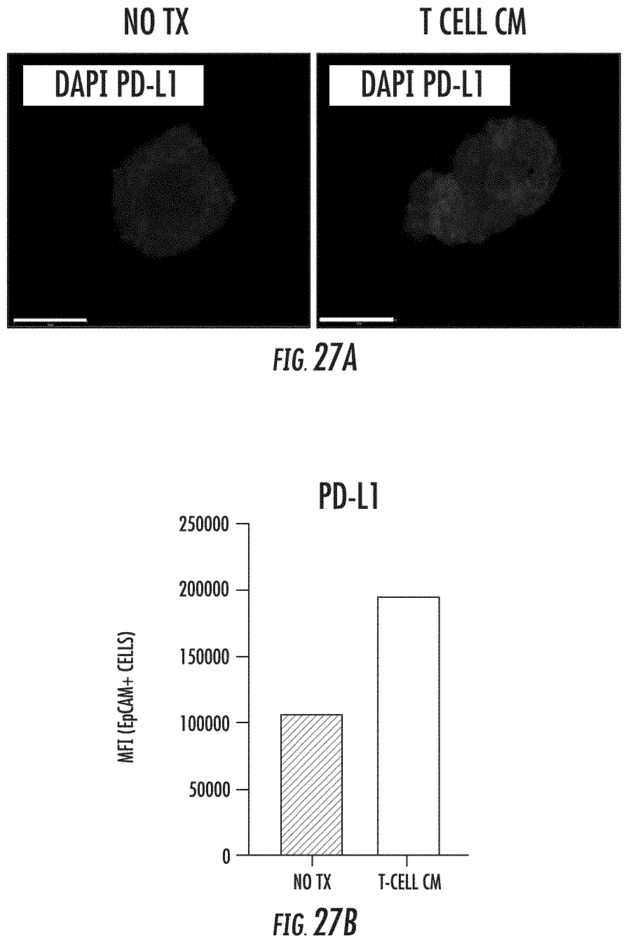

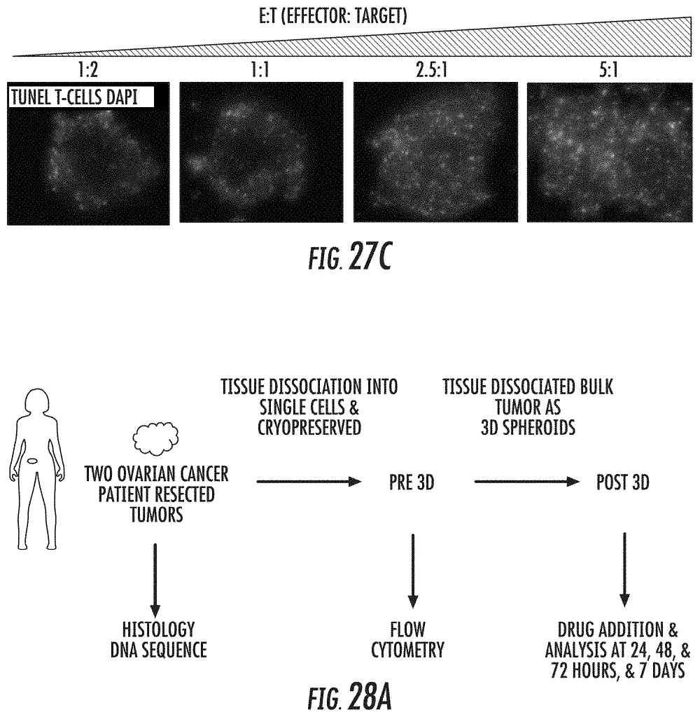

FIGS. 27A-C show induced changes in PD-L1 expression and detection of T-cell mediated apoptosis. Ovarian primary cancer cells were cultured with autologous TILs for 72 hours with or without T-cell CM and subsequently fixed and stained for PD-L1 (FIG. 27A) or dissociated and analyzed via flow cytometry (FIG. 27B). An increase in PD-L1 expression was observed via immunofluorescence and flow cytometry. (FIG. 27C) To confirm changes in viability were T-cell mediated, healthy donor T-cells labeled with Cell Tracker Deep Red were co-cultured with a melanoma cell line for 4 hours, fixed, and then stained with TUNEL to detect apoptotic cells. Increasing amount of TUNEL positive cells were observed with increased E:T ratios.

The imaging analysis platform presents an alternative method for detecting T-cell mediated cell death in response to I/O agents. The platform allows for the rapid analysis of multiple timepoints to probe I/O drug kinetics in the system. By labeling immune cells, it is possible to study the I/O drug's effect specifically on T-cells which is something conventional methods, such as CellTiter-Glo® lack. Applicant was able to detect changes in immune composition of primary cancer cells cultured in 3D, demonstrating a functional and responsive system. Further, it can be confirmed that cells cultured in Applicant's platform are responsive and can induce changes in PD-L1 expression, a biomarker for immune escape. In addition, TUNEL staining demonstrates that the tumor cell death observed is due to the presence of T-cells in the system.

### Example 11—PD-1/PD-L1 Checkpoint Inhibitors in Combination with Olaparib Display Antitumor Activity in Ovarian Cancer Patient Derived Three-Dimensional Spheroid Cultures

Immune checkpoint inhibitors (ICIs) that target programmed cell death protein 1 (PD-1) and programmed death-ligand 1 (PD-L1) have shown modest activity as monotherapies for the treatment of ovarian cancer (OC). The rationale for using these therapies in combination with poly (ADP-ribose) polymerase inhibitors (PARP-Is) has been described and their in vivo application will benefit from ex vivo platforms that aid in the prediction of patient response or resistance to therapy. This example determined the effectiveness of detecting patient-specific immune-related activity in OC using three-dimensional (3D) spheroids. Immune-related cell composition, PD-1 and PD-L1 expression status was evaluated using cells dissociated from fresh OC tissue from two patients prior to and following 3D culture. The patient sample with the greatest increase in the proportion of PD-L1 positive cells also possessed more activated cytotoxic T-cells and mature CD11c+ dendritic cells (DCs) compared to the other patient sample. Upon cytokine stimulation, patient samples demonstrated increases in cytotoxic T-cell activation and DC major histocompatibility complex (MHC) class-II expression. Pembrolizumab increased cytokine secretion, enhanced olaparib cytotoxicity, and reduced spheroid viability in a T-cell dependent manner. Furthermore, durvalumab and olaparib combination treatment increased cell death in a synergistic manner. This work demonstrates that immune cell activity and functional modulation can accurately be detected using our ex vivo 3D spheroid platform, and it presents evidence for their utility to demonstrate sensitivity to ICIs alone or in combination with PARP-Is in a pre-clinical setting.

Ovarian cancer (OC) is the leading cause of death for women with gynecologic cancer in the United States. Surgical debulking followed by chemotherapy are the standard of care for OC, yet most patients become resistant to chemotherapy resulting in a five-year survival rate below 50%. To elicit long-term disease remission, both the incorporation of new therapies into the current treatment paradigm and personalized testing methods to define which patient gets which therapy are under considerable investigation.

Immunotherapy has revolutionized the treatment of many solid tumors, and there is a rationale for their use in the treatment of OC. OC patients with tumor-infiltrating lymphocytes (TILs) display a significant improvement in five-year survival rates compared to patients without TILs. The positive correlation between OC survival and immune cell recruitment to the tumor microenvironment provides compelling evidence that anti-tumor immune surveillance is an important determinant for OC clinical outcomes and suggests the immunogenic nature of OC could be exploited as a treatment option by using immune checkpoint inhibitors (ICIs) such as those that target programmed cell death protein 1 (PD-1) and programmed death-ligand 1 (PD-L1). Unfortunately, the reports of OC patient response to ICI therapy have generally been underwhelming. It is unclear if this drug class is simply ineffective against OC or if the lack of preclinical research to date is hindering the translation of ICI efficacy to the clinic.

Poly(ADP-ribose) polymerase inhibitors (PARP-Is) have shown impressive clinical activities for OC patients. However, intrinsic and acquired resistance often limit their effectiveness as monotherapies. The role PARP-Is play as immune modulators to enhance checkpoint blockade efficacy has recently emerged. A phase I/II clinical trial demonstrated that the PARP-I, niraparib, in combination with pembrolizumab produced complete or partial responses in 18% of patients with recurrent platinum-resistant OC compared to less than a 5% response rate with niraparib alone. Further understanding of the immune modulatory capacity of anti-PD-1/PD-L1 inhibitors alone and in combination with PARP-Is will enhance our knowledge in what drives sensitivity for different solid tumor indications, such as OC.

In efforts to extend immunotherapy research efforts to a broader range of solid tumors, Applicant modified an existing ex vivo OC 3D spheroid assay to detect the potential synergy between anti-PD-1/PD-L1 inhibitors, pembrolizumab or durvalumab, in combination with the PARP-I, olaparib. This work builds upon those studies to test immune modulatory agents through the inclusion and characterization of autologous immune cells. Immune composition and function were evaluated prior to monitoring therapy related changes in 3D spheroid phenotypes and viability. Finally, overall patient-specific differences in immune composition and drug response were determined.

### Methods

Generation of 3D Spheroids

Live OC tissue was received within 24 hours of surgery and dissociated to single cells via mechanical and enzymatic digestion. Cells were then cryopreserved until ready for use. 3D spheroids were generated as previously described. Briefly, cells were seeded in KIYA-PREDICT™ media (KIYATEC, Inc., South Carolina, USA) in 384-well round bottom, ultra-low attachment plates (Corning Inc., New York, USA) and centrifuged at 500×g for five minutes then placed in a 37° C. incubator at 5% CO₂.

3D Spheroid Drug Response Assay

Given the prevalence of drug resistance and altered drug penetration for 3D cultures, drug concentrations for single dose experiments were in the micromolar or microgram per milliliter range for testing. For pembrolizumab and olaparib combination studies, 100 μg/mL pembrolizumab (SelleckChem, Texas, USA), 50 μM or 100 μM olaparib (MedChemExpress, New Jersey, USA) for OVC33 or OVC45 respectively, were added alone or together along with KIYA-PREDICT™ media as no treatment control or 0.2% DMSO as vehicle control. Viability was determined 48 hours later. For durvalumab (Selleckchem, Texas, USA) and olaparib combination studies, spheroids were treated with olaparib for 48 hours, followed by durvalumab for an additional 72 hours. For direct pembrolizumab treatment of T-cells, CD3 positive cells were separated from dissociated bulk tumor cells using the EasySep CD3 Positive Selection kit II (StemCell Technologies, Vancouver, Canada) and incubated in the presence or absence of 300 μg/mL pembrolizumab in order to saturate all PD-1 sites. T-cells were then added to the bulk cells and seeded for 3D spheroid culture. Viability was determined 48 hours later. Viability read outs were conducted using CellTiter-Glo® 3D Cell Viability Assay (Promega, Wisconsin, USA) and relative luminescence units (RLUs) were recorded using a TECAN infinite M1000pro (Mannedorf, Switzerland).

Flow Cytometry

3D spheroids were resuspended and incubated in ACCUTASE™ (StemCell Technologies, Vancouver, Canada) to facilitate spheroid dissociation. Dissociated cells were washed in PBS and resuspended in FACs buffer (2% FBS, 2 mM EDTA, in PBS). Antibodies and dilutions used are listed below in Table 3. Antibodies were added and incubated for 10 minutes at 4° C. Samples were washed, centrifuged then resuspended in FACs buffer. DRAQ 7 (BD Pharmingen™, New Jersey, USA) dead cell dye was added for dead cell detection and exclusion. Samples were analyzed using the CytoFLEX LX flow cytometer and software (Beckman Coulter, California, USA). Percent of parent was graphed and evaluated for statistics using GraphPad Prism (GraphPad Software, California, USA).

TABLE 3
Antibodies used in present study

| | | Catalog | Appli- | |
|---|---|---|---|---|
| Antibody | Supplier | Number | cation | Dilution |
| CD45-FITC | Miltenyi | 130-110-769 | FCM | 1:100 |
| PD-L1-PE | BD | 557924 | FCM | 1:20 |
| | Biosciences | | | |
| EpCAM-PerCP-Vio700 | Miltenyi | 130-111-120 | FCM | 1:100 |
| CD3-APC | Miltenyi | 130-113-135 | FCM | 1:100 |
| CD4-FITC | Miltenyi | 130-114-531 | FCM | 1:100 |
| CD8-PerCP-Vio700 | Miltenyi | 130-110-682 | FCM | 1:100 |
| CD25-PE | Miltenyi | 130-115-534 | FCM | 1:100 |
| CD69-APC-Vio770 | Miltenyi | 130-112-616 | FCM | 1:100 |
| PD-1-PE-Vio770 | Miltenyi | 130-117-698 | FCM | 1:100 |
| CD11c-APC-Vio770 | Miltenyi | 130-114-111 | FCM | 1:100 |
| HLA-DR-APC | Miltenyi | 130-111-943 | FCM | 1:100 |
| CD103-PE | Miltenyi | 130-111-985 | FCM | 1:100 |
| REA control FITC | Miltenyi | 130-113-437 | FCM | 1:100 |
| mouse IgG1k PE | BD | 555749 | FCM | 1:20 |
| | Biosciences | | | |
| REA control (S) | Miltenyi | 130-113-441 | FCM | 1:100 |
| PerCP-Vio700 | | | | |
| REA control (S) APC | Miltenyi | 130-113-434 | FCM | 1:100 |
| REA control (S) FITC | Miltenyi | 130-113-437 | FCM | 1:100 |
| REA control (S) | Miltenyi | 130-113-441 | FCM | 1:100 |
| PerCP-Vio700 | | | | |
| REA control (S) PE | Miltenyi | 130-113-438 | FCM | 1:100 |
| REA control (S) | Miltenyi | 130-113-435 | FCM | 1:100 |
| APC-Vio770 | | | | |
| mouse IgG2b PE-Vio770 | Miltenyi | 130-096-825 | FCM | 1:20 |
| REA control (S) | Miltenyi | 130-113-435 | FCM | 1:100 |
| APC-Vio770 | | | | |
| REA control (S) APC | Miltenyi | 130-113-434 | FCM | 1:100 |
| REA control (S) PE | Miltenyi | 130-113438 | FCM | 1:100 |
| pan Cytokeratin | Abeam | ab86134 | IHC | 1:500 |
| CD11c | Abeam | ab52632 | IHC | 1:50 |
| PD-1 | Abeam | ab170190 | IHC | 1:100 |
| CD8 | Abeam | ab17147 | IHC | 1:50 |
| PD-L1 | Abeam | ab210931 | IHC | 1:1000 |
| mouse IgG1 kappa | Abeam | ab91353 | IHC | 1:50 |
| recombinant rabbit IgG | Abeam | ab172730 | IHC | 1:50 |
| mouse IgG1 kappa | Abeam | ab170190 | IHC | 1:100 |
| mouse IgG2a kappa | Abeam | ab18415 | IHC | 1:1000 |
| CD8 | Abeam | ab4055 | IF | 1:100 |
| CD11c | Abeam | ab254183 | IF | 1:50 |
| PD-L1 | Abeam | ab205921 | IF | 1:200 |
| Alexa fluor 594 | Life | A11012 | IF | 1:500 |
| | Technologies | | | |
| Alexa fluor 488 | Life | A11029 | IF | 1:500 |
| | Technologies | | | |

Table 3 Continued. Antibodies used in present study

Immunohistochemistry

Upon receipt of fresh tissue, a portion was removed and immediately fixed in formalin for 48 hours and processed as previously described. The fixed tissue was embedded in paraffin and sections (10 μm) were mounted onto glass slides. Following hematoxylin and eosin staining, slides were cover slipped using Permount medium. Otherwise, rehydration and antigen retrieval were performed using citrate buffer pH 6.0 (Abcam, Cambridge, UK) or Tris-EDTA buffer pH 9.0 (Abcam, Cambridge, UK) according to antibody specifications. Antibodies and dilutions used are listed in Table 3. Antibody staining was visualized using mouse and rabbit specific HRP/DAB IHC Detection Kit-Micro-polymer (Abcam, Cambridge, UK). Brightfield images were imaged at 40× using EVOS M7000 microscope and Invitrogen™ EVOS™ M7000 Imaging System (Thermo Fisher Scientific, Massachusetts, USA).

Immunofluorescence

Spheroids were fixed using 3.7% formaldehyde. Spheroids were washed in FACs buffer then cytospun to adhere cells to glass slides. Cells were permeabilized using 0.3% Triton X-100 in PBS, incubated in blocking buffer (0.1% bovine serum albumin, 0.2% Triton X-100, 10% goat serum and 0.05% Tween 20 for one hour followed by primary antibody in a humidifier at 4° C. overnight. Antibodies and dilutions used are listed in Table 3. Following primary antibody incubation, cells were washed with blocking buffer then incubated with secondary antibodies in the dark for one hour. Cells were washed with blocking buffer; nuclei were stained, and slides were mounted with a cover slip using Fluoroshield mounting medium with DAPI (Abcam, Cambridge, UK).

Cytokine Stimulation

T-cell conditioned media (T-cell CM) was used as a source of cytokines to stimulate immune related functions. Separated CD3 positive cells were expanded in ImmunoCult-XF T-cell Expansion Medium (StemCell Technologies, Vancouver, Canada) according to manufacturer's recommendations. Briefly, for the initiation of T-cell expansion, ImmunoCult Human CD3/CD28 T-cell Activator (StemCell Technologies, Vancouver, Canada) was added to growth medium with 10 ng/mL interleukin-2 (IL-2) (Sigma, Missouri, USA). Expanded T-cells were pelleted, and the T-cell CM was aliquoted and stored at −20° C. For 3D spheroid stimulation, spheroids were formed overnight, and the T-cell CM was added at a 1:1 ratio the following day. 3D spheroids were stimulated with T-cell CM for 48 or 72 hours.

Cytokine Detection

Human Discovery Immunotherapy Fixed Panel Magnetic Luminex performance assay was purchased from R&D Systems (Minnesota, USA). Supernatant was collected at day three of 3D spheroid culture and stored at −80° C. Samples were processed according to manufacturer's recommendations and the assay plate was run on Bio-Rad Luminex® BioPLEX200™ System (Bio-Rad Laboratories, California, USA). Analyte concentration was determined by interpolation of assay standards using GraphPad Prism (GraphPad Software, California, USA). Fold change analyte secretion was determined for pembrolizumab, olaparib, combination by comparing them to vehicle control samples.

Statistical Analysis

Statistical analysis was performed using GraphPad Prism software version 8.2.1. Results are expressed as the mean±standard deviation (SD). Unpaired t tests were used to determine significance between two groups. Unpaired One-way ANOVA with multiple comparisons was used to determine significance across three or more groups. Combenefit software generated concentration responses and Loewe synergy indices from calculated percent viability normalized to vehicle control.

### Results

Patient Tumor Tissues Display a Non-Desert Phenotype.

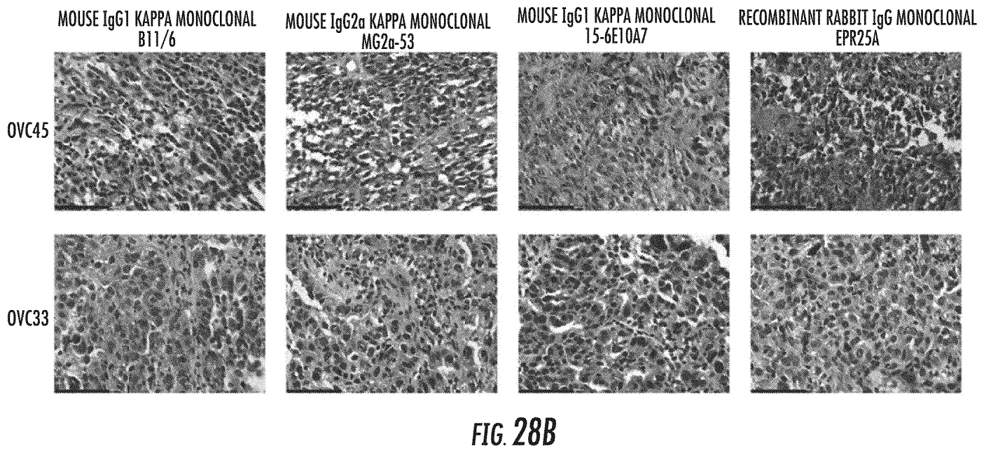

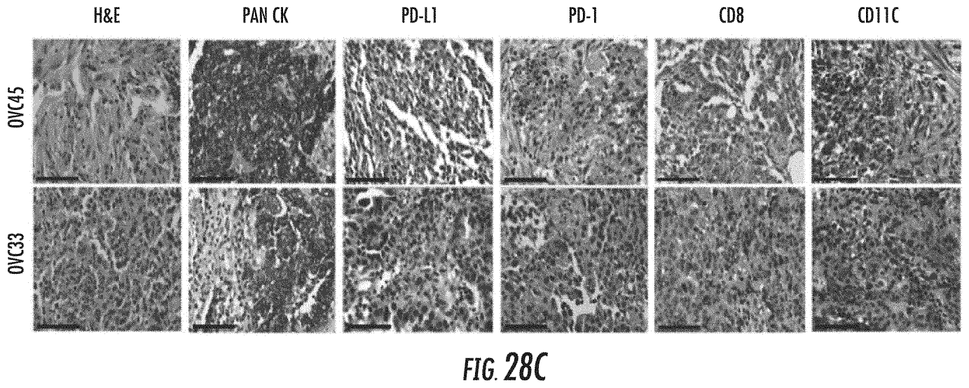

Two newly diagnosed treatment naïve serous OC patient samples that were matched in stage (IIIC) and grade (high) were chosen for testing. OC patient samples were characterized from tissue resection through 3D spheroid culture. Cells were characterized following 3D spheroid culture (Post 3D) and compared back to the original cell composition found Pre 3D (FIG. 28A). Histological analysis of the OC tissues verified the immune composition for the two patient samples tested, OVC45 and OVC33 (FIG. 28A, 28B (control), and 28C). Both samples were composed primarily of tumor cells as identified by pan cytokeratin staining. PD-1 positive and CD8 positive cells were observed distributed throughout both tissues. CD11c+ dendritic cells (DCs) were found in both tested patient samples. While OVC33 stained more positive for PD-L1 expression compared to OVC45, the staining in general was very diffuse and faint. Given detection of both cytotoxic T-cells and DCs, both tissues were classified as non-desert.

An Increased Proportion of PD-L1 Positive Tumor Cells is Detected Following Ex Vivo 3D Spheroid Culture.

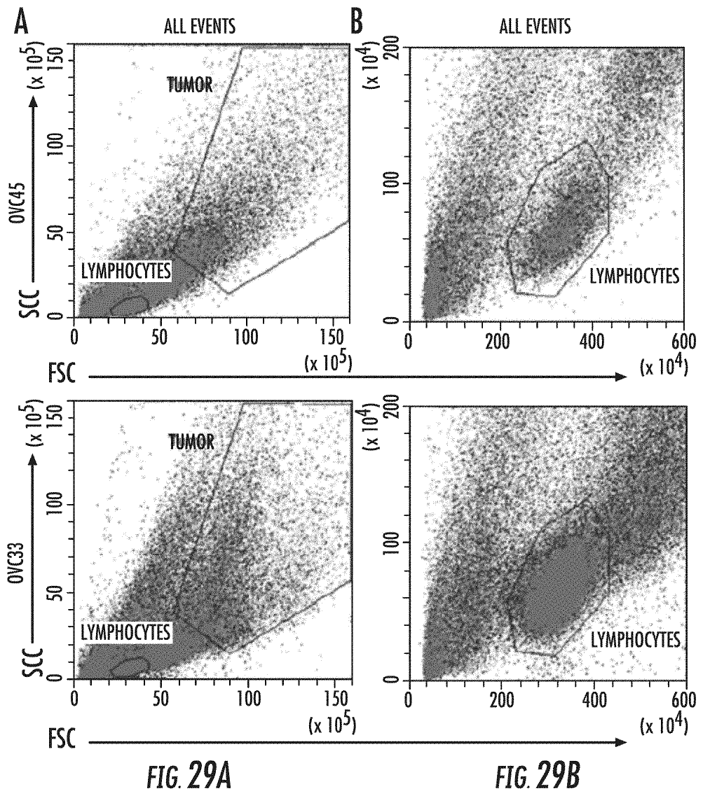

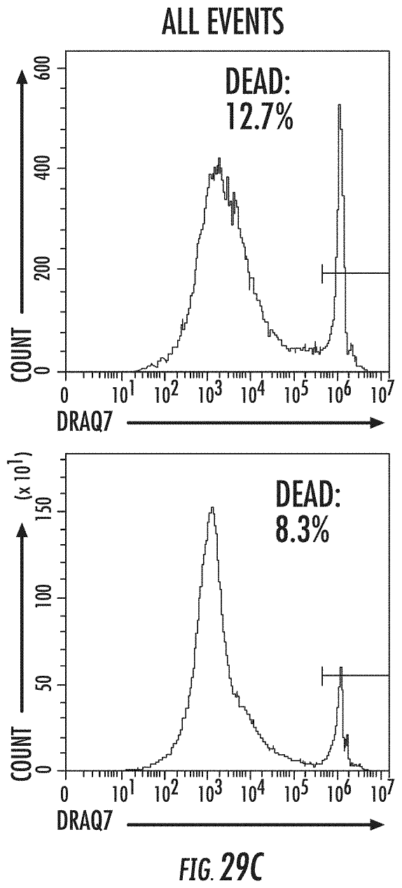

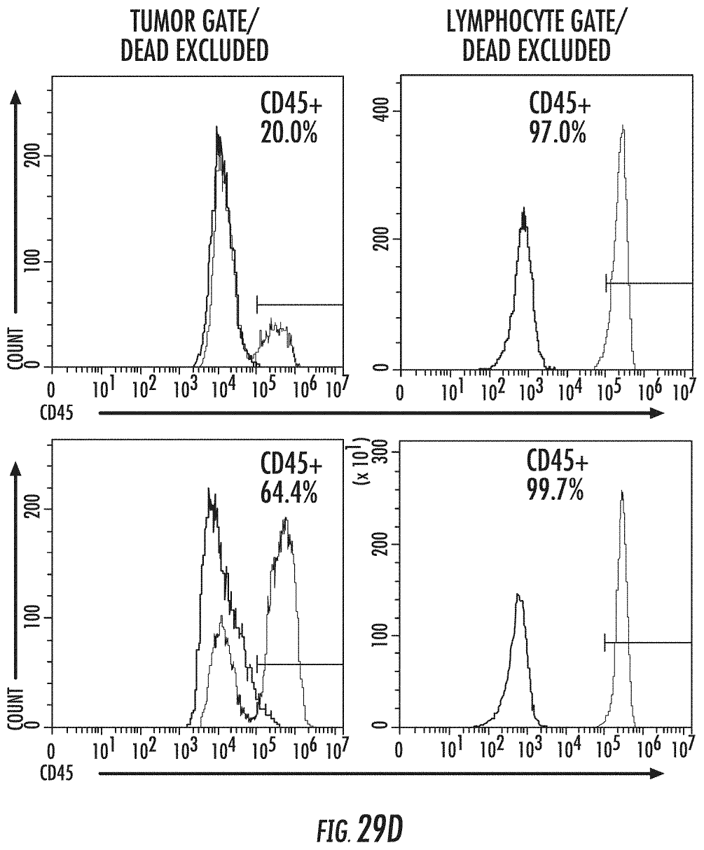

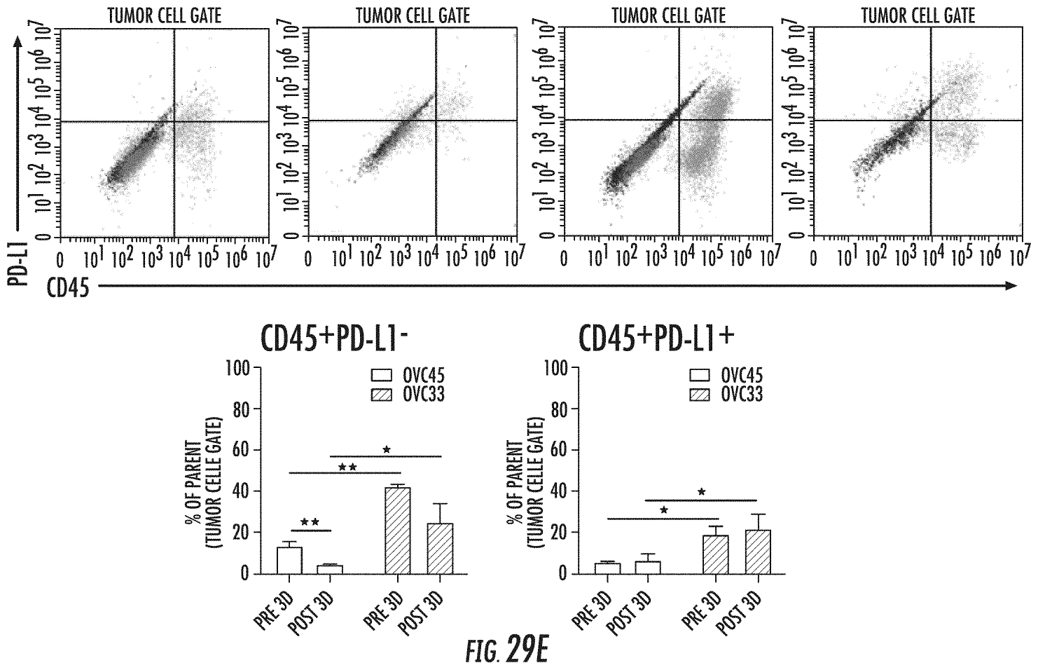

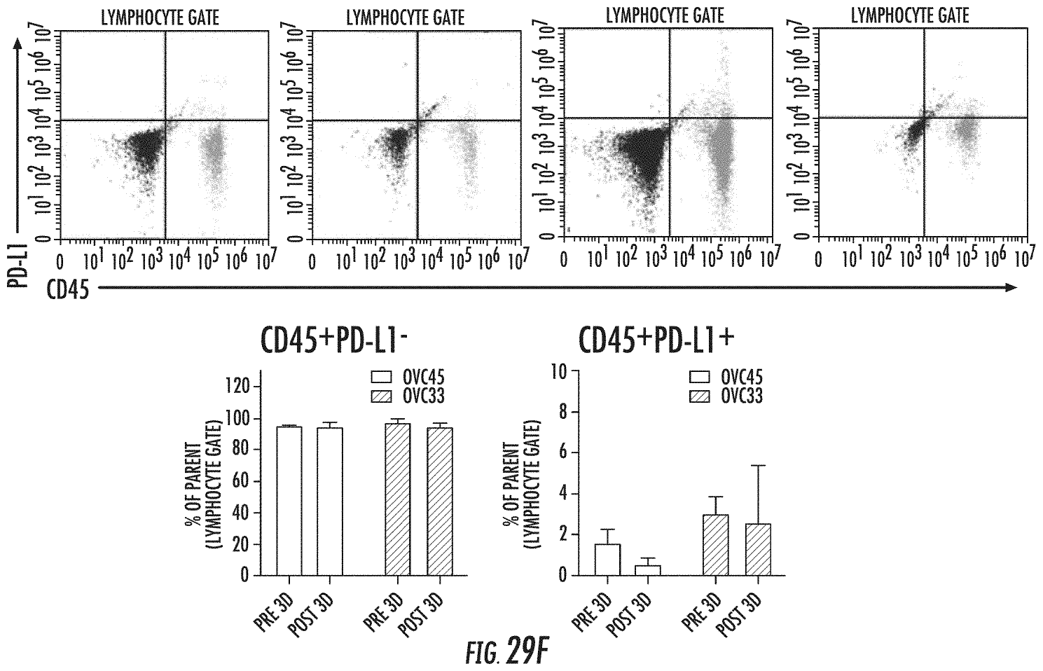

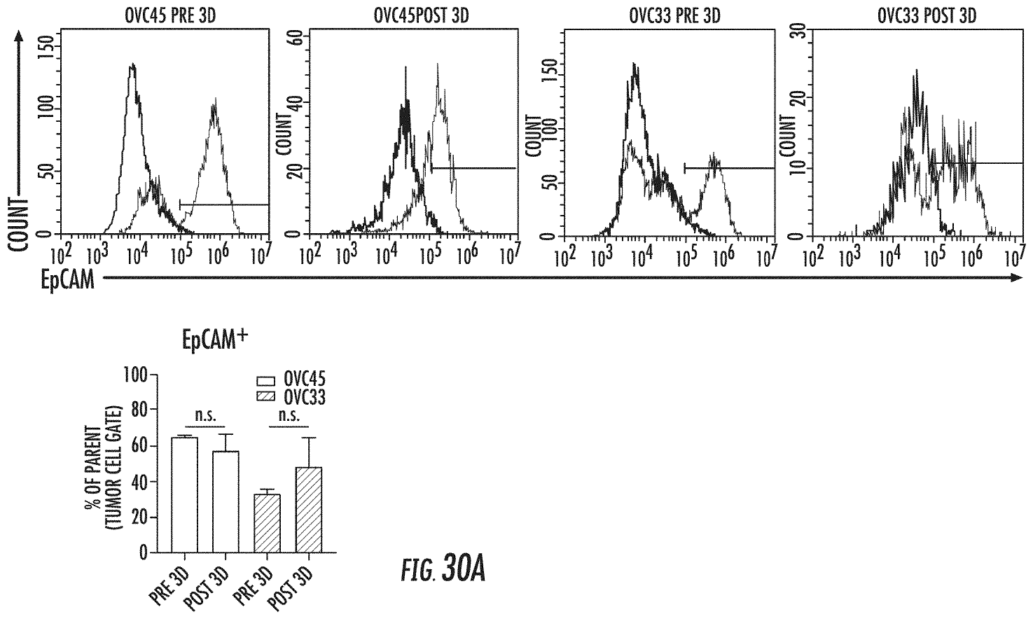

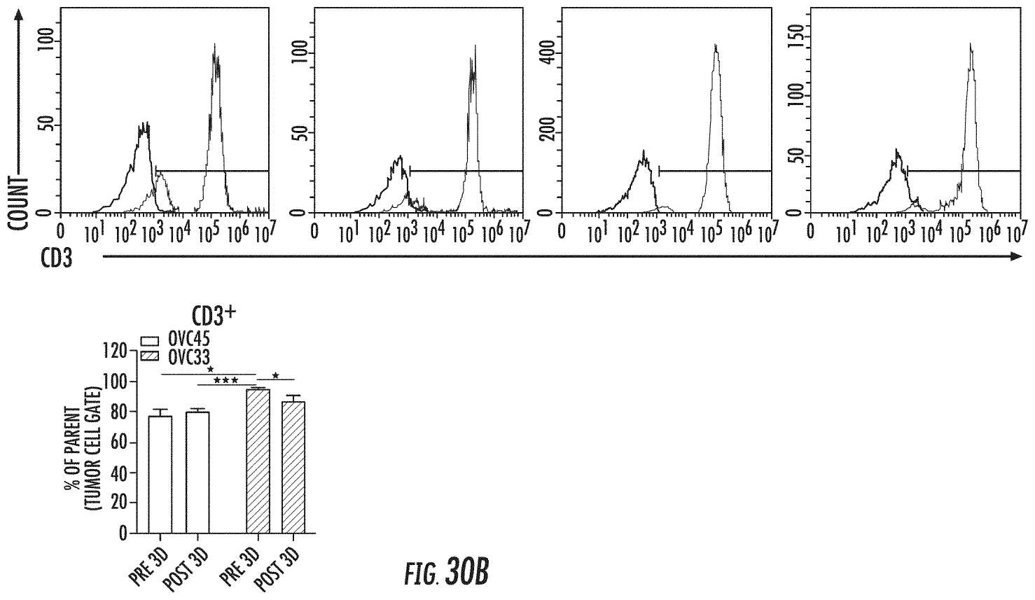

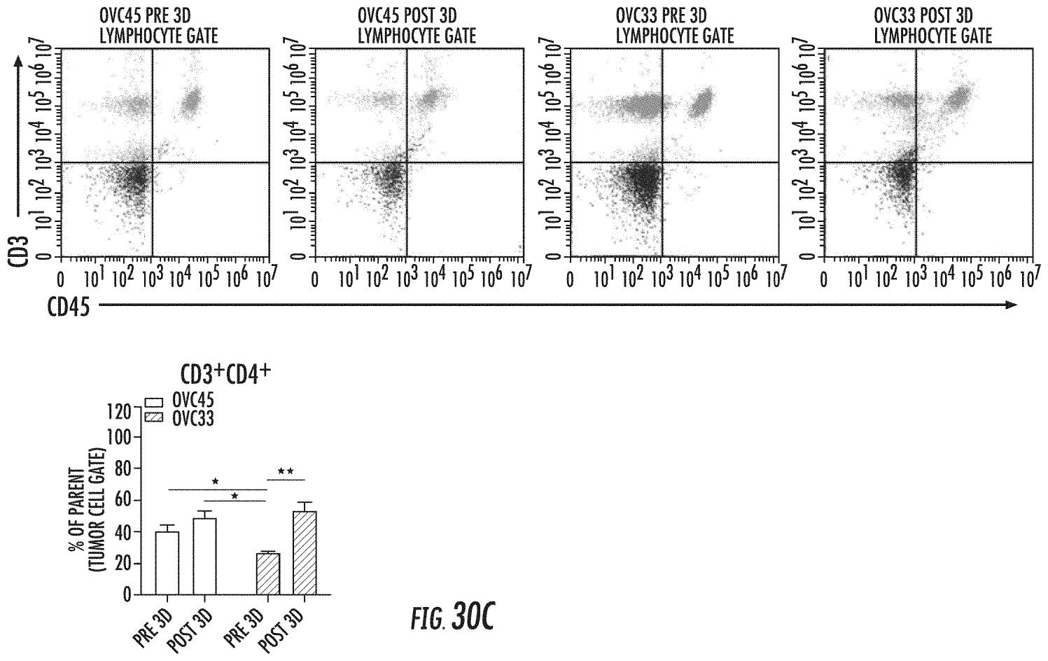

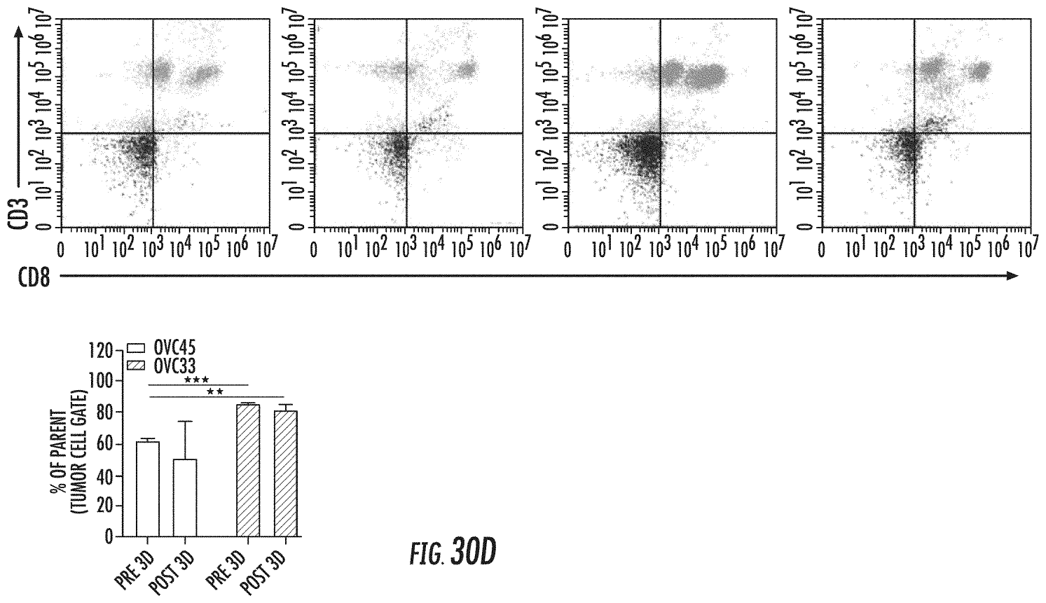

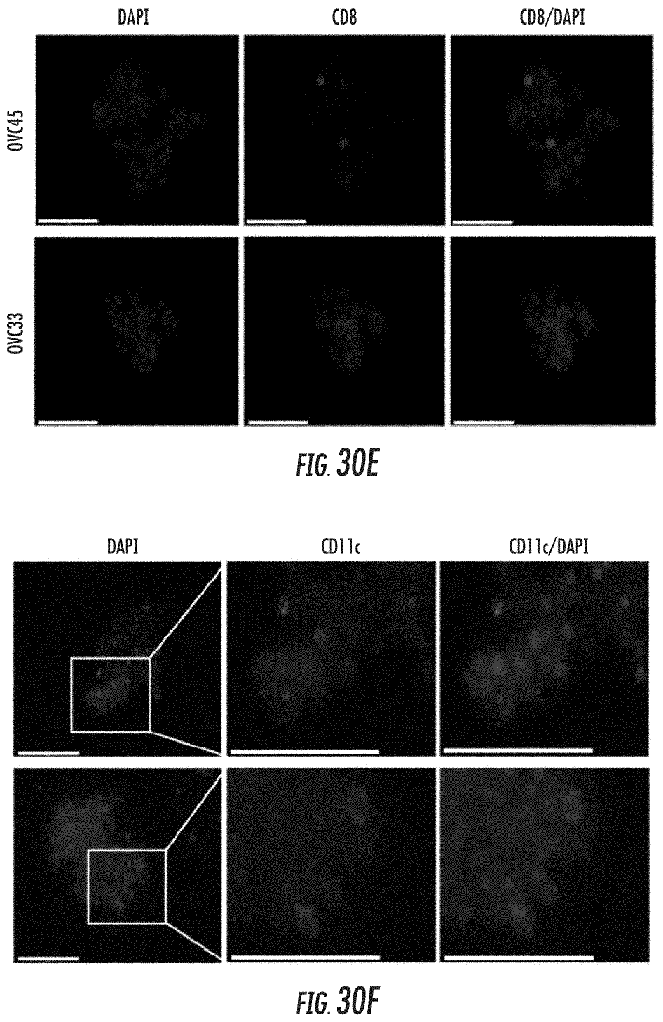

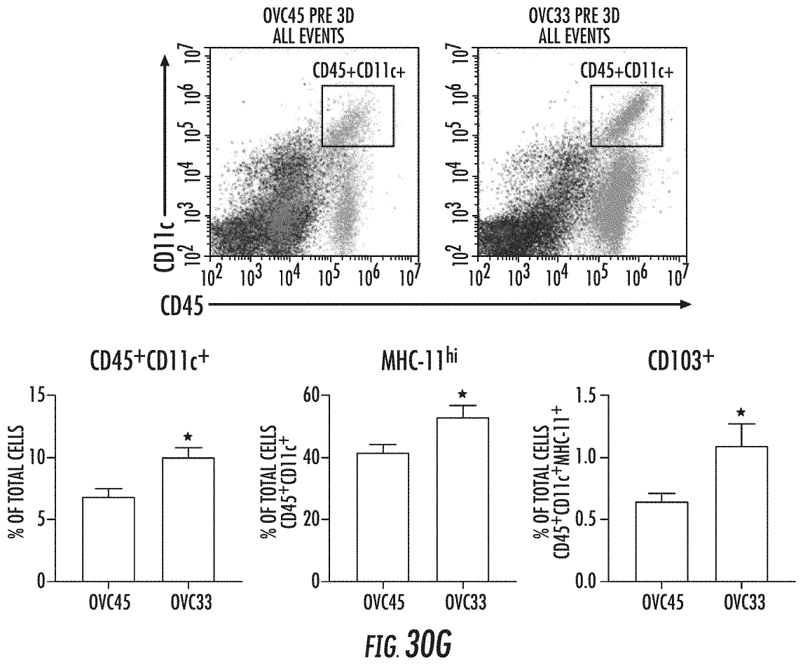

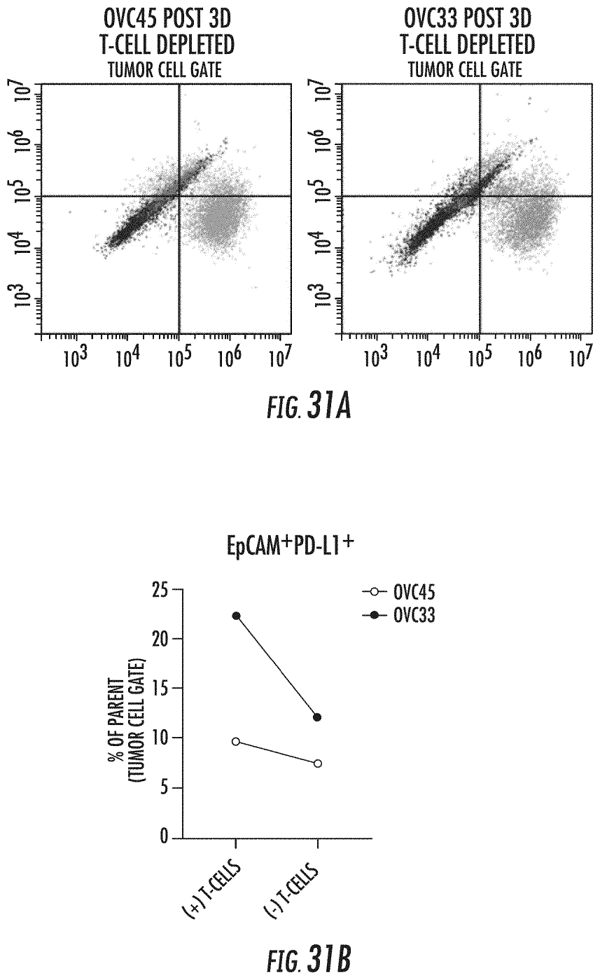

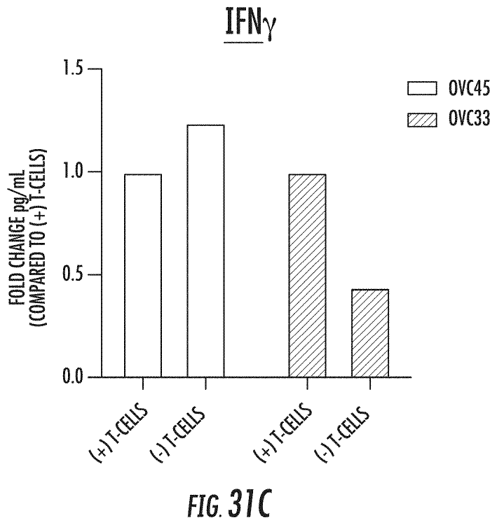

To determine patient specific similarities and the effects of 3D cell culture, Pre 3D cellular composition and Post 3D cellular composition were compared. The proportion of live tumor cells were assessed via flow cytometry by EpCAM expression after dead cell exclusion within a gating region defined as tumor cell gate (FIGS. 29C and 29D). There was no significant change in the proportion of total EpCAM positive cells Post 3D (FIG. 30A). OVC45 had greater Pre 3D EpCAM positive cells that were found to be PD-L1 negative compared to OVC33 (FIG. 30A). Interestingly, both patient samples had significant increases in PD-L1 positive tumor cells following 3D spheroid culture with OVC33 demonstrating the greatest increase in this population. The impact that T-cells have on PD-L1/EpCAM dual positive cells was tested by culturing OVC45 and OVC33 following T-cell depletion using CD3 positive selection (FIGS. 31A-31C). It was found that there were approximately twice as few dual positive cells detected for OVC33 when cultured in the absence of T-cells. A decrease in IFNγ levels Post 3D for OVC33 when T-cells were depleted was also detected, but this was not observed for OVC45. These data suggest the presence of T-cells may have an impact on the microenvironment within our 3D cell culture platform. These results may be indicative of the PD-L1 positive tumor cell population being less vulnerable within the 3D spheroid system compared to the PD-L1 negative tumor cell counterpart.

When evaluating the immune population, after dead cell exclusion, live CD45 positive cells were detected in both the large gate defined for tumor cells as well as within the smaller lymphocyte gate (FIGS. 29A-29B). OVC33 had significantly more Pre 3D immune cells compared to OVC45 (FIG. 29E). Approximately 20% of the cells that stained positive for CD45 within the large gate for OVC33 were also found to be PD-L1 positive. The majority (greater than 95%) of lymphocytes identified within the small gate were found to be PD-L1 negative for both patient samples (FIG. 29F).

Next, the inter-patient proportion of T-cell subpopulations was determined. An evaluation of total CD3 positive cells revealed only small shifts in relative amounts with 3D spheroid culture (FIG. 30B). However, OVC45 had significantly more helper T-cells (CD3+CD4+) compared to OVC33 Pre 3D (FIG. 30C) with no change in the proportion of CD4 positive T-cells following 3D culture for OVC45, but a significant increase for OVC33. Upon characterization of cytotoxic T-cells (CD3+CD8+), observed no significant change following 3D culture for either patient samples was observed, but it was determined that OVC33 had significantly more CD8 positive T-cells compared to OVC45 (FIG. 30D). The presence of CD8 positive T-cells in both tissues following 3D culture was confirmed using immunofluorescence (FIG. 30E) and it was found that OVC33 had clusters of CD8 positive cells which is a morphological phenotype associated with activated T-cells.

Since T-cell activation has been shown to be regulated by DCs in the tumor immune microenvironment (TIME), and DCs have been shown to play a critical role in ICI efficacy, the presence of tumor associated DCs for both patient samples was examined. To confirm that the spheroid system maintains DCs, the presence of DCs for both OVC45 and OVC33 following 3D culture was detected (FIG. 30F). A greater abundance of DCs within OVC33 (FIG. 30G) was found and this population was found to express higher levels of MI-IC class-II (WW-II) indicating higher antigen-presenting machinery and the marker CD103 which is found on DCs with a potent stimulatory impact on effector T-cell priming. In total, these results demonstrate that immune-related patient-specific differences can be detected within our 3D spheroid system and may shift through the course of 3D cell culture in a patient-specific manner.

Differential Immune Cell Populations are Detected within Ex Vivo 3D Spheroids

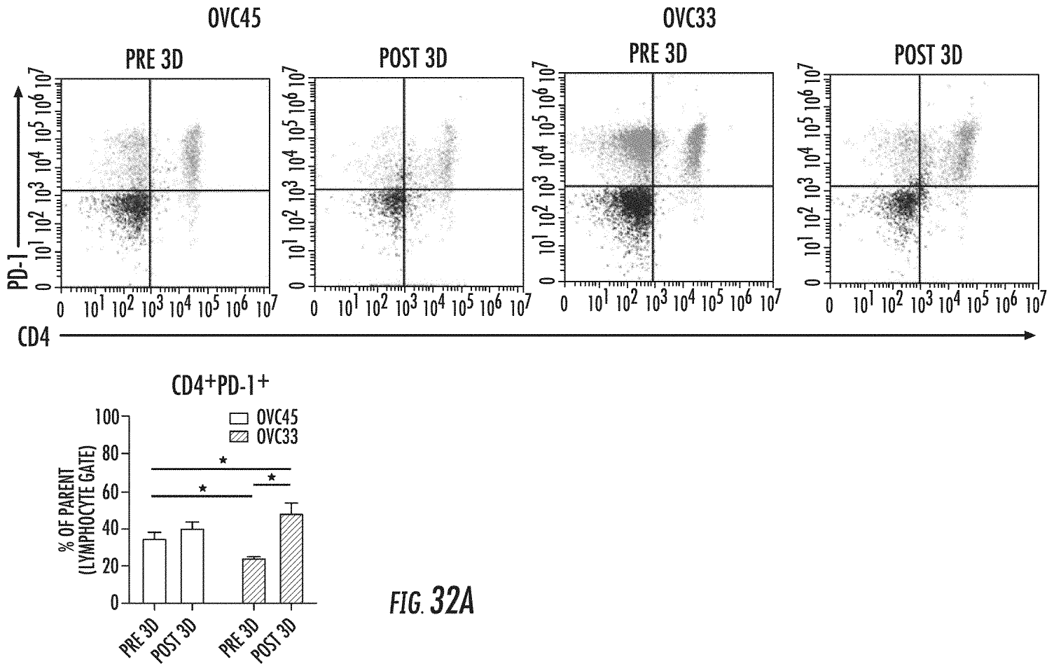

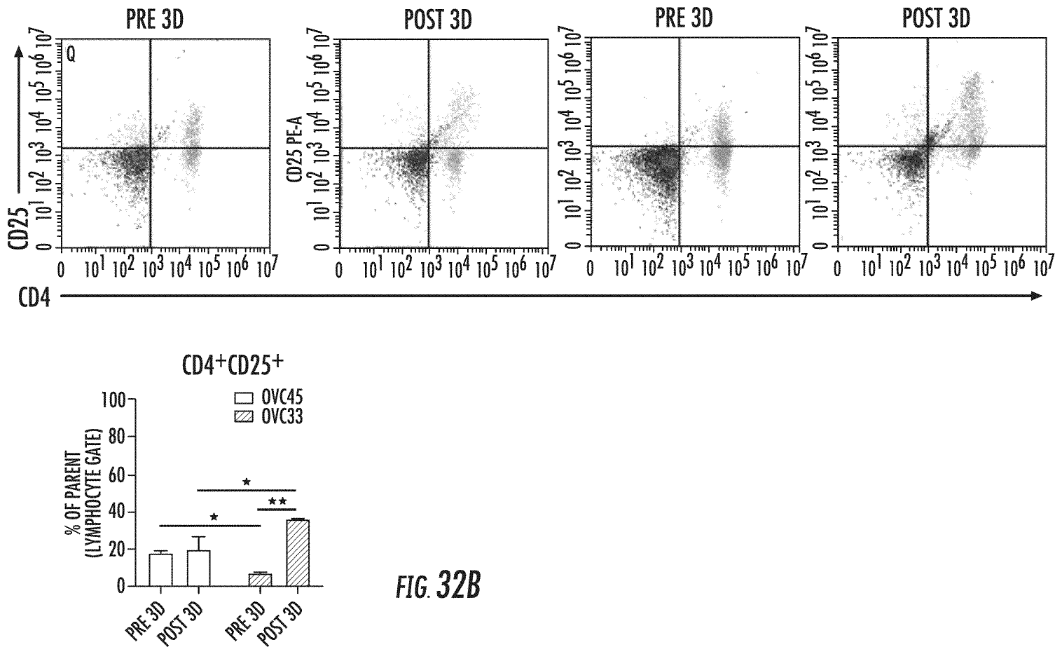

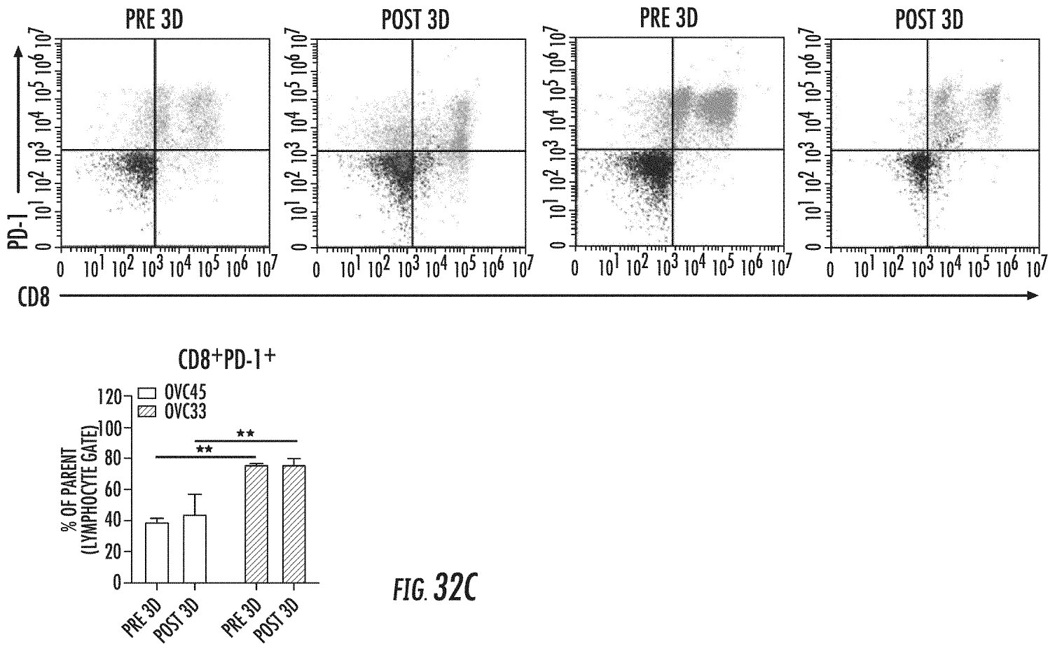

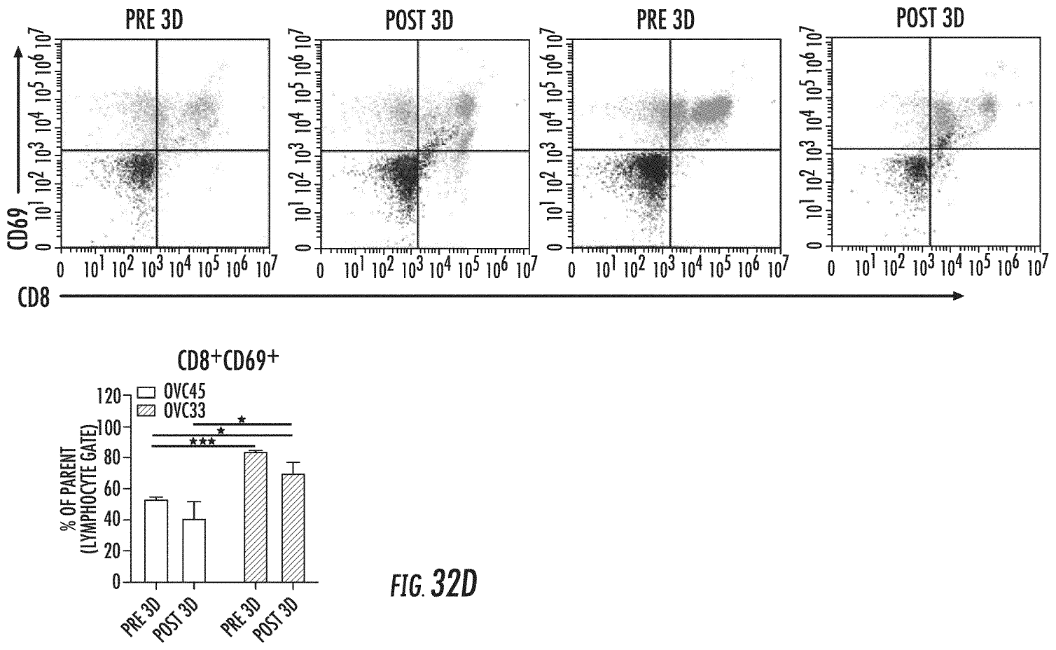

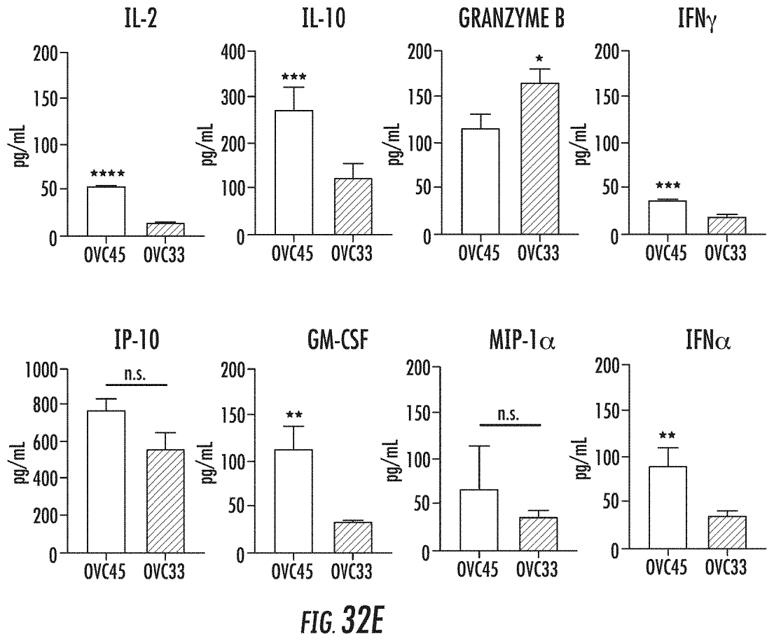

Next, the different T-cell populations for markers of activation was characterized. OVC45 had greater CD4 positive cells Pre 3D that were positive for PD-1 (FIG. 32A) or found to be Tregs (CD4+/CD25+) compared to OVC33 (FIG. 32B). Conversely, OVC33 had more CD8 positive T-cells that were positive for PD-1 (FIG. 32C) as well as more activated cytotoxic T-cells (CD8+/CD69+) compared to OVC45 (FIG. 32D). The patient-specific T-cell populations were proportionally stable in 3D culture for OVC45 while OVC33 had a significant increase in PD-1 positive CD4 positive T-cells and Tregs.

The presence of cytokines known to be secreted by immune-related cells was also examined (FIG. 32E). OVC45 had significantly greater amounts of IL-2, IL-10, and IFNγ compared to OVC33, however there was no significant difference in the amount of IFNγ-induced protein 10 (IP-10). Interleukin-10 (IL-10) is an immune-suppressive cytokine known to be produced by Tregs, and despite the observed increase in Tregs by OVC33 Post 3D, the proportion of Tregs in OVC45 Pre 3D was greater than that of OVC33 (FIG. 32B). These results suggest the Pre 3D immune composition may be a better reflection of the detected cytokine secretion. OVC33 had significantly greater granzyme B compared to OVC45 which may reflect the higher proportion of activated cytotoxic T-cells found in OVC33 both Pre 3D and Post 3D (FIG. 32D). The presence of granulocyte-macrophage colony-stimulating factor (GM-CSF) indicates T-cell activation for both patient samples. Macrophage inflammatory protein-1 alpha (MIP-1a) and tumor necrosis factor alpha (TNFα) were detected in both samples which provides evidence that macrophages may be present in the 3D spheroid system.

Immune Cell Function can be Enhanced Through Cytokine Stimulation in Ex Vivo 3D Spheroids

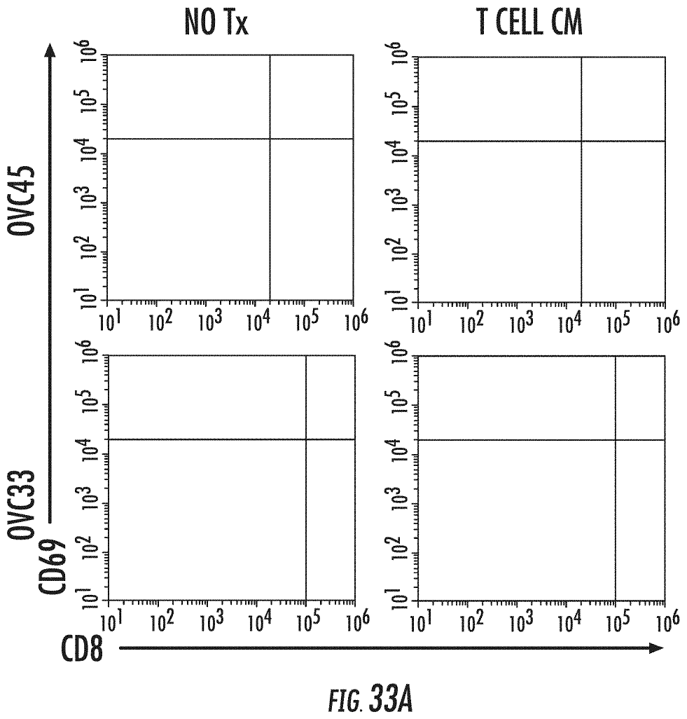

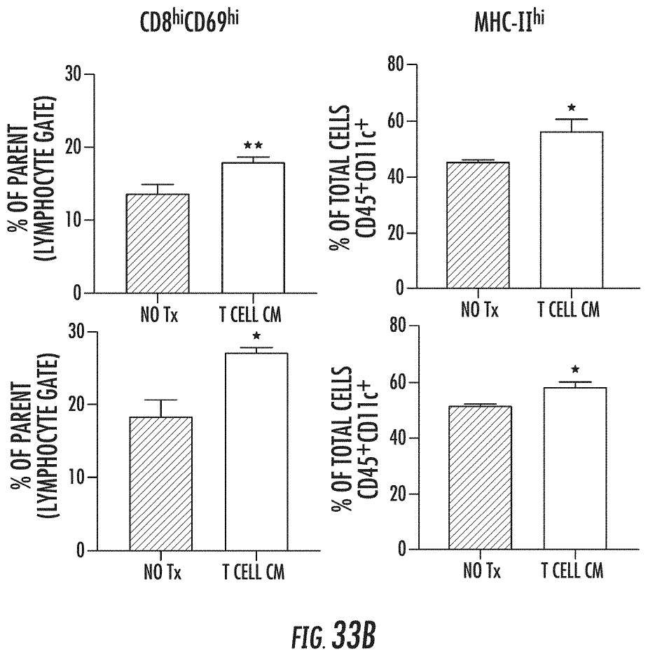

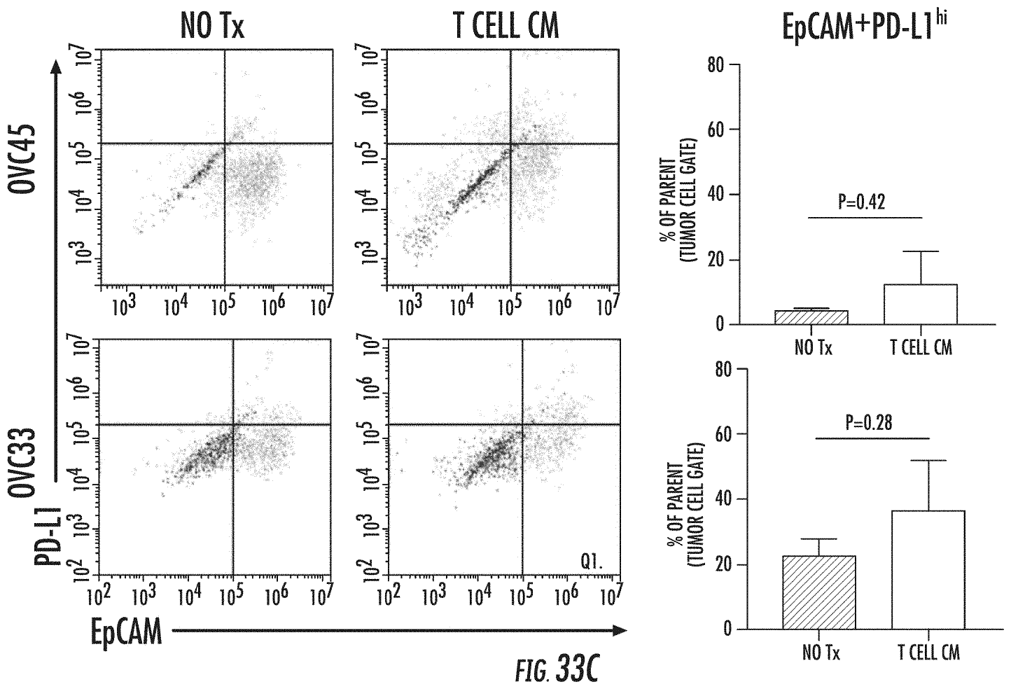

To demonstrate modulation of immune cell function in ex vivo 3D spheroid culture, the spheroids were cultured in conditioned T-cell expansion medium (T-cell CM). When T-cells are activated, they rapidly divide and secrete key cytokines to promote immune responses. Conditioned medium from the expansion of primary OC TILs was therefore utilized as a source of cytokines for stimulation. By using this cytokine cocktail, it was possible to evaluate different cell types and different activation mechanisms following a single treatment. Significant increases for both patient samples were observed when comparing activated cytotoxic T-cells (CD8hiCD69hi) following T-cell CM treatment (FIG. 33A). IFNγ has been shown to increase MHC-II expression on DCs in vitro. Therefore, Applicant examined whether T-cell CM could increase MHC-II expression within our 3D spheroid system and detected an increase in MHC-II expression on DCs from both samples (FIG. 33B). Given the detected increases in T-cell activation and DC maturation, Applicant determined if T-cell CM affected PD-L1 expression. An increase in PD-L1 expression for was detected for both tissues after T-cell CM treatment. To determine if this increase in PD-L1 expression was associated with tumor cells specifically, the EpCAM+/PD-L1+ cell population was measured with an upward trend in dual EpCAM+/PD-L1+ cells for both tissues (FIG. 33C). These results demonstrate that the immune cells are active in our ex vivo 3D spheroid cultures, and their function can be enhanced through treatment modulation.

Pembrolizumab Alters T-Cell Function, Enhances Olaparib Efficacy, and Induces T-Cell Dependent Reduction in Spheroid Viability.

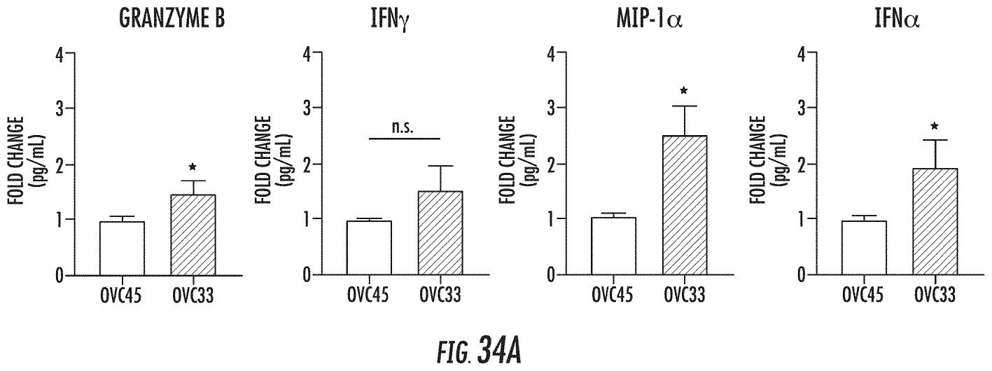

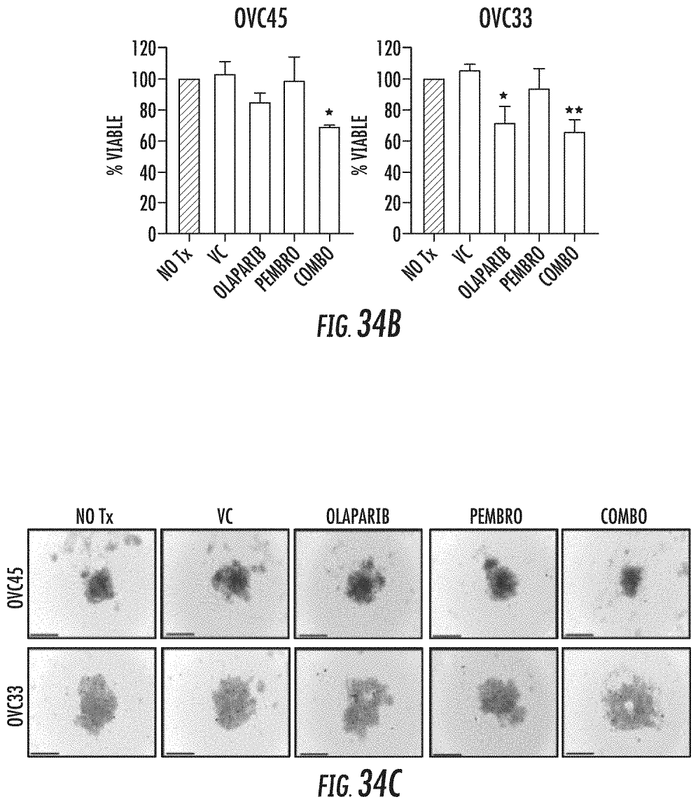

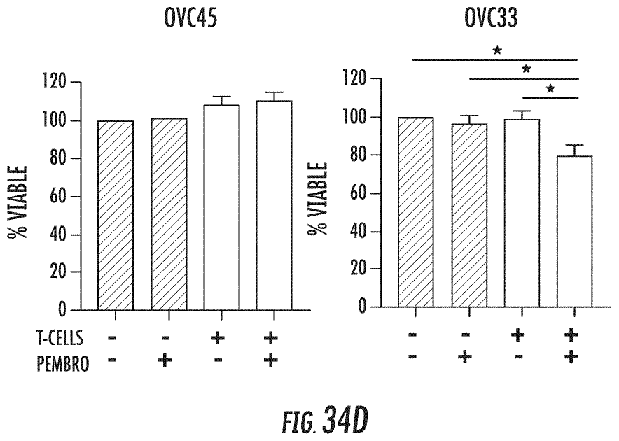

Changes in cytokine secretion and cell viability following treatment with pembrolizumab were examined. No changes in cytokines were detected in OVC45 while OVC33 showed increases in granzyme B, MIP-1α, and TNFα (FIG. 34A). Applicant next tested pembrolizumab and the PARP-I, olaparib, alone or in combination for the reduction of spheroid viability. A decrease in spheroid viability with pembrolizumab treatment alone was not detected for both samples (FIG. 34B). OVC33 was more sensitive to olaparib alone compared to OVC45, and reduction in 3D spheroid viability for OVC45 occurred only when treated with a combination of pembrolizumab and olaparib. Applicant detected a reduction in spheroid size for OVC45 following combination treatment. OVC33 spheroids appear less dense with less cell contact following olaparib or combination treatment (FIG. 34C). To enhance pembrolizumab efficacy, Applicant tested direct incubation of the T-cells with pembrolizumab prior to 3D spheroid incorporation via T-cell separation from the Pre 3D bulk cell suspension. For these experiments, it was our intension to saturate all PD-1 sites. Our maximum testing concentration of pembrolizumab was selected since pembrolizumab demonstrates a relatively high half-life (approximately 27 days) ultimately resulting in a gradual approach to steady state in vivo. An intravenous dosing frequency of 10 mg/kg once every two weeks has a predicted pembrolizumab maximum serum concentration of approximately 200 μg/mL for advanced solid tumor cancer patients. OVC33 had a significant reduction in spheroid viability only when spheroids were treated with T-cells incubated with pembrolizumab (FIG. 34D). This result demonstrates that pembrolizumab treatment can reduce spheroid viability and that its efficacy is T-cell dependent.

Durvalumab and Olaparib Synergistically Reduce OVC33 Spheroid Viability.

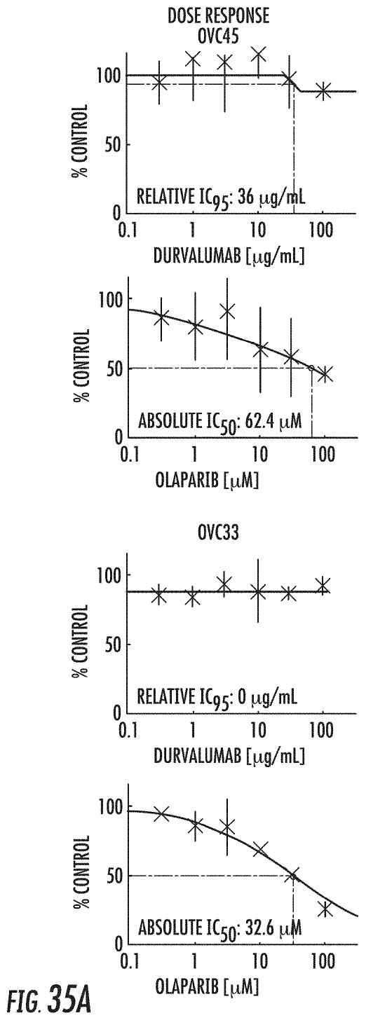

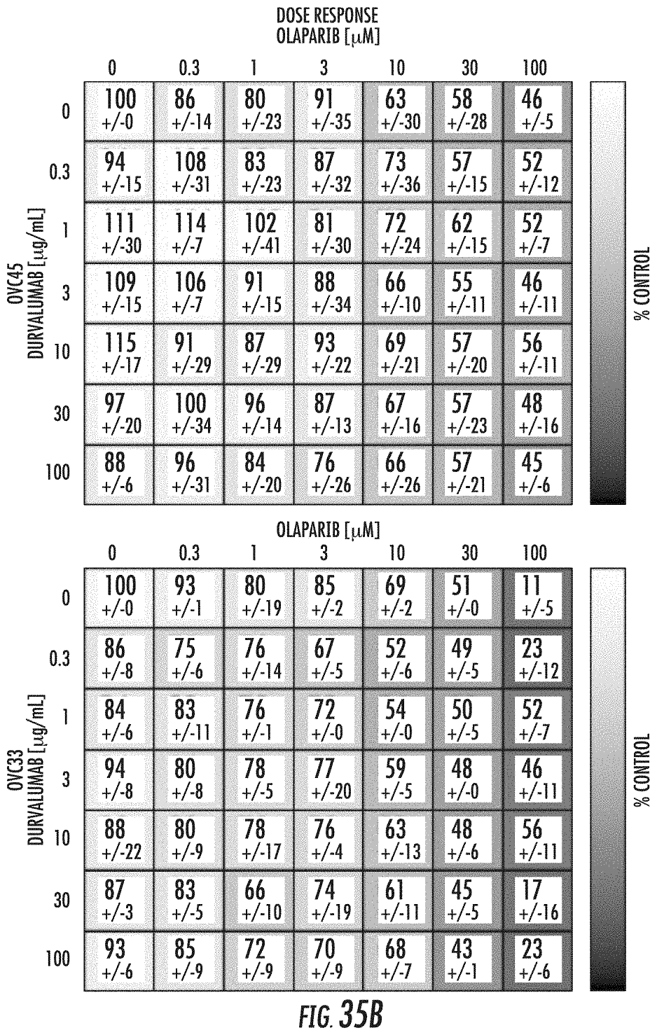

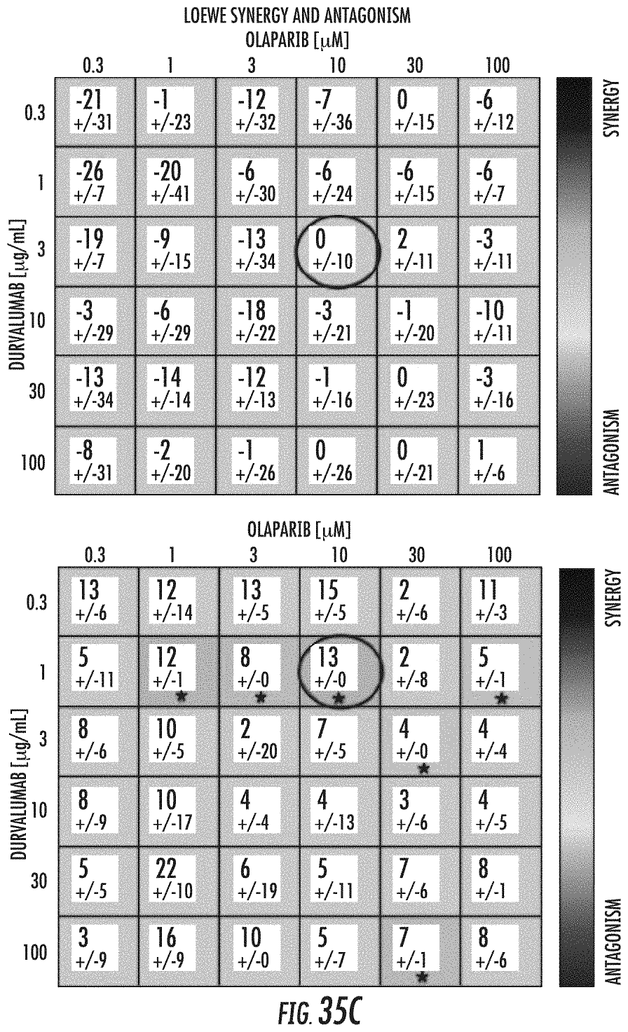

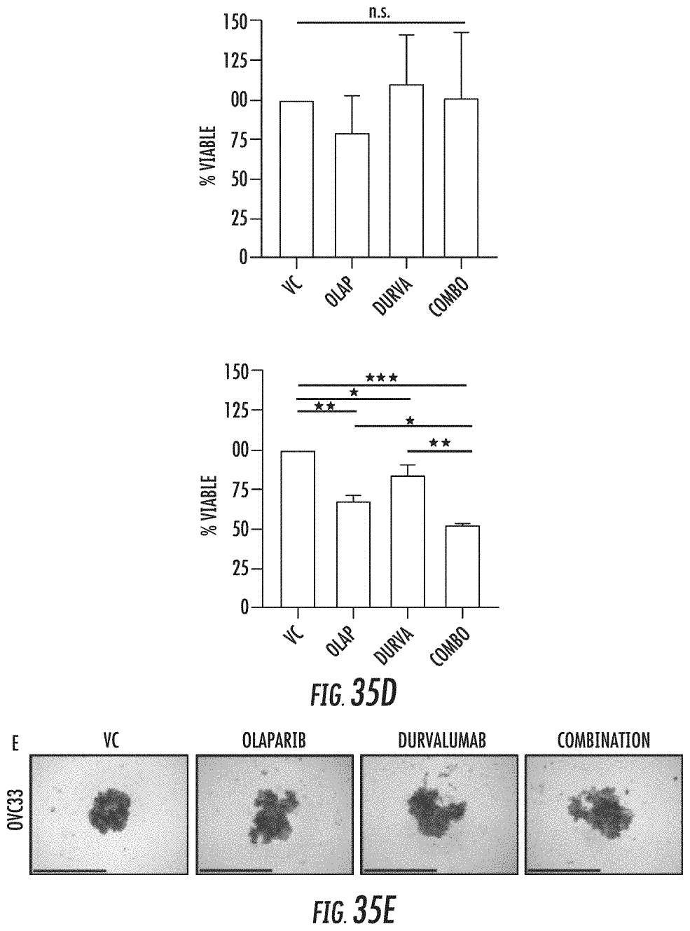

Since enrichment of PD-L1 positive tumor cells was detected for both tested samples, Applicant next decided to evaluate the sensitivity of these samples to the anti-PD-L1 antibody durvalumab. Applicant tested durvalumab in combination with olaparib in a sequential dosing strategy to better mimic clinical dosing strategies and determine if drug order has an impact on ICI/PARP-I combination studies. OVC33 remained more sensitive to single agent olaparib than OVC45 (FIG. 35A). Durvalumab treatment alone did not result in a dose-dependent reduction in spheroid viability. Applicant compared the cross dose-response of both drugs and also determined if the percent viability following treatments were synergistic (FIG. 35B). Six combination treatments were deemed significantly synergistic for OVC33 (FIG. 35C). Significant synergy was detected at 10 μM olaparib and 1 μg/mL durvalumab (FIG. 35D). Applicant did not detect a significant change in spheroid viability for OVC45 following this same drug treatment (FIG. 35E). The half-life of durvalumab is high resulting in predicted achievable serum concentration levels of greater than 10 μg/mL with a twice weekly intravenous dosing regimen. These data suggest our findings are could be clinically achievable. Representative images of OVC33 show decreased spheroid density and a loss of cell compactness and cell contact following combination therapy (FIG. 35E). Ultimately, synergistic efficacy between durvalumab and olaparib treatment can be detected using our 3D spheroid culture and the response is patient specific.

### DISCUSSION

In this example, it has been demonstrated that patient-specific, non-expanded, autologous tumor cells and immune cells can be incorporated ex vivo into 3D spheroids and monitored over the course of a week for changes in immune cell composition, activation, cytokine secretion, and drug response. The most significant changes included an increase in the EpCAM+/PD-L1+ population and shifts in Tregs. There were significant differences in the immune cell composition between the two patient samples that were reflected in their cytokine secretion profiles and responses to olaparib, pembrolizumab, and durvalumab. This data shows the ability of our ex vivo 3D spheroid platform to model patient-specific response to PARP-I/ICI combination therapy in conjunction with each patient's tumor-immune microenvironment.

Effective immunotherapy requires understanding the TIME as it is what often drives therapy response. Taking advantage of the relationship between the quality and character of the TIME and response to immunotherapy has been proposed as a personalized approach for the treatment of cancer. Generally, OC demonstrates low to modest somatic mutational burden which may explain the overall limited antitumor activity detected with ICI monotherapy in the clinic. Yet if the OC TIME is immunologically “hot” or T-cell inflamed, there is a moderate to high probability of response to anti-PD-1/PD-L1 treatment. Recent reports show that OC positive for PD-L1 expression correlated with higher response to pembrolizumab. In our study, the patient sample with the highest PD-L1 expression, OVC33, significantly responded to anti-PD-1/PD-L1 treatment and in a T-cell dependent manner. OVC33's TIME composition may reflect T-cell exhaustion and dysfunction. OVC33 had lower levels of detected cytokines compared to OVC45 and although OVC33 had more activated cytotoxic T-cells, they were predominately high PD-1 expressors. The low levels of cytokine secretion by OVC33 was found to be reversible since pembrolizumab was able to increase cytokine levels. Given its T-cell profile, CD103 positive DC population, and PD-L1 positivity, OVC33 may have the “right” immune composition to be reinvigorated by a PD-1/PD-L1 inhibitor. More patient samples will have to be evaluated within our 3D model to make any potential correlation between Pre 3D immune composition and response to an ICI.

PARP-Is can induce synthetic lethality in BRCA1/2 deficient OC. Interestingly, non-BRCA1/2 mutant OC that are classified as possessing “BRCAness” qualities respond to PARP-Is. Further studies will need to be executed to identify the relationship between BRCA1/2 mutational status and BRCAness phenotypes to the response of ICIs in our 3D spheroid system. Olaparib has been reported to increase immune cell infiltration. Due to limited control of self-assembling during spheroid formation, it is believed that Applicant's other 3D culture models, such as microtumor models, are more ideal to monitor therapy induced immune cell infiltration. Our spheroid model also alters the original distribution of the tumor immune landscape. Yet, Applicant proposed that it is the TIME character and functional capabilities, not solely the spatial arrangement that may be reflective of therapeutic response.

Despite the limitation of 3D spheroid models, the advantages they provide for preclinical research are compelling. Many groups have shown the advantages of spheroid models, but they often rely on incorporation of allogenic immune cells from healthy donors such as peripheral blood mononuclueated cells. The phenotype of tumor-associated immune cells have been shown to be functionally different from those found in the periphery. Our model incorporates all cells found within the patient's TIME. Future work will explore the role of these other cell types from a more in depth perspective.

While the TIME and biomarkers such as PD-1/PD-L1 provide some information as to the ability of a patient's tumor to respond to ICI therapies, many patients still do not respond in the clinic. This is likely due to the fact that the patient's tumor cells were never directly tested in conjunction with the selected therapy for a direct response prediction. This study provides proof of concept data for the ability of our ex vivo 3D spheroid models to measure response to both single agent and combination PARP-I and ICI through the specific, direct interaction between a patient's cells and the ICI of choice.

### CONCLUSION

This work furthers efforts to expand in vitro testing of immune-oncology agents and ex vivo based detection methods of ICI sensitivity. The need for combination therapies to overcome monotherapy resistance often limits ICI utility for many tumor types. Applicant hopes to harness the power of patient-specific TIME to identify signatures relating cell composition and function to therapy response to find biomarkers that predict drug sensitivity.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A method of treating cancer comprising co-culturing immune cells and tumor cells isolated from a subject under conditions that allow the immune cells and the tumor cells to form a cell mass, exposing the cell mass to at least one therapeutic agent, and measuring the responsiveness of the tumor cells in the cell mass to the at least one therapeutic agent.

2. The method of claim 1, further comprising determining a ratio of the immune cells to the tumor cells prior to co-culturing the immune cells and the tumor cells.

3. The method of claim 2, wherein the ratio of the tumor cells to the immune cells is 1:0.05 to 1:100.

4. The method of claim 3, wherein the ratio of the tumor cells to the immune cells is 1:10.

5. The method of claim 1, wherein the immune cells and the tumor cells are from the same subject.

6. The method of claim 1, wherein the immune cells comprise T cells, natural killer cells, dendritic cells, macrophages, or a combination thereof.

7. The method of claim 6, wherein the immune cells comprise T cells.

8. The method of claim 1, wherein the at least one therapeutic agent comprises at least one checkpoint inhibitor.

9. The method of claim 8, wherein the checkpoint inhibitor targets PD-1.

10. The method of claim 1, wherein the at least one therapeutic agent comprises at least one poly(ADP-ribose) polymerase inhibitor.

11. The method of claim 10, wherein the at least one poly(ADP-ribose) polymerase inhibitor comprises olaparib, niraparib, rucaparib, talazoparib, or a combination thereof.

12. The method of claim 1, wherein the cell mass is a tumor spheroid.

13. The method of claim 1, wherein the responsiveness of the tumor cells is a decrease in viability.

14. The method of claim 13, further comprising identifying a patient-specific treatment based on the decrease in tumor cell viability.

15. The method of claim 1, wherein the at least one therapeutic agent induces secretion of TNF-α, MIP-1α, and INFγ.

16. The method of claim 1, further comprising isolating immune cells from the formed cell mass and further expanding the immune cells for use in a cell therapy.

17-55. (canceled)

56. The method of claim 1, wherein the immune cells and the tumor cells are from different subjects.

57. The method of claim 1, wherein the immune cells are from a healthy subject.

58. The method of claim 1, wherein the immune cells express an immune checkpoint protein selected from the group consisting of CTLA4, BTLA, LAG3, ICOS, PD-1, PDL1, KIR, CD40, OX40, CD137, GITR, CD27 and TIM-3.

59. The method of claim 58, wherein, wherein the immune checkpoint protein is PD-1.
